(19) United States
(12) Patent Application Publication
(10) Pub. No.: US 2022/0218607 A1
(43) Pub. Date: Jul. 14, 2022

(54) STABLE, LOW-VISCOSITY ANTIBODY FORMULATIONS AND USES THEREOF

(71) Applicant: SANOFI, Paris (FR)

(72) Inventors: Atul SALUJA, Bridgewater, NJ (US); Manjori GANGULY, Bridgewater, NJ (US); Kelvin REMBERT, Bridgewater, NJ (US); Yatin GOKARN, Bridgewater, NJ (US)

(21) Appl. No.: 17/604,890

(22) PCT Filed: Apr. 23, 2020
(86) PCT No.: PCT/EP2020/061340

§ 371 Date: Oct. 19, 2021

Related U.S. Application Data
(60) Provisional application No. 62/837,518, filed on Apr. 23, 2019.

(30) Foreign Application Priority Data
Feb. 17, 2020 (EP) 20305145.3

(51) Int. Cl. A61K 9/08; C07K 16/28

(52) U.S. Cl. CPC A61K 9/08; C07K 2317/71; C07K 16/2866; C07K 16/2803

(57) ABSTRACT

Provided herein are aqueous antibody formulations that exhibit improved stability and low viscosity. The formulations include an antibody or an antigen-binding fragment, a buffer, and a salt selected from the group of magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, or lithium sulfate, where the formulations have a pH of about 4 to about 8 and, optionally an osmolality of about 250 mOsm/kg to about 1500 mOsm/kg

### TECHNICAL FIELD

The present invention relates generally to antibody formulations. Specifically, the present invention relates to monoclonal antibody formulations with improved stability and low viscosity.

### BACKGROUND

Antibody formulations can lose their efficacy over time due, for example, to the effects of denaturation, oxidation, aggregation, or other degradation reactions. Degradation and aggregation of antibodies in an antibody formulation can also pose risks such as toxicity or immunogenicity. High solution viscosity can negatively impact the manufacturability and performance of protein therapeutic agents, especially those formulated at high protein concentrations.

The low level of stability exhibited by currently available pharmaceutical antibody formulations is disadvantageous due to, for example, loss of efficacy of the antibody formulation before administration and possible toxicity and immunogenicity due to the degradation/aggregation. Traditional excipients used to stabilize proteins in solution can often increase the viscosity of the solution. Therefore, there is a need in the art for an antibody formulation that will allow for improved stability of antibodies. Additionally, there is a need in the art of antibody formulation that will allow for the development of stable and low-viscosity antibody solutions.

### SUMMARY

The present invention is based on the discovery that antibodies can be stabilized in solution by including a salt selected from the group of magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate. The resulting stabilized antibody solutions are also less viscous compared to formulations which do not include one of these salts or that contain traditional excipients, for example sugars like sucrose, used to stabilize proteins. The osmolality of stabilized antibody solutions with one of these salts is also less than that with sugars and polyols such as sucrose, trehalose, sorbitol, mannitol etc. when formulated at equipotent concentrations.

In view of this discovery, provided herein are aqueous antibody formulations that include about 0.1 mg/mL to about 500 mg/mL of an antibody or an antigen-binding fragment thereof (e.g., any of the exemplary antibodies or antigen-binding fragments described herein or known in the art); about 1 mM to about 100 mM of a buffer (e.g., any of the exemplary buffers described herein or known in the art); and about 1 mM to 750 mM of a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, where the formulations have a pH of about 4 to about 8.

Also provided are an aqueous antibody formulations that include about 0.1 mg/mL to about 500 mg/mL of an antibody or an antigen-binding fragment thereof (e.g., any of the exemplary antibodies or antigen-binding fragments described herein or known in the art); and about 1 mM to 750 mM of a salt selected from the group of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, where the formulations have a pH of about 4 to about 8. In some embodiments of the aqueous antibody formulations, the aqueous antibody formulation is a buffer-free aqueous antibody formulation.

Some embodiments of any of the aqueous antibody formulations described herein can further include a stabilizer (e.g., any of the exemplary stabilizers described herein or known in the art) and/or a surfactant (e.g., any of the exemplary surfactants described herein or known in the art). Also provided are injection devices that include any of these formulations, and kits including one or more vials containing any of these formulations.

Also provided are methods of making an aqueous antibody formulation that include mixing or combining: (i) an antibody or an antigen-binding fragment thereof (e.g., any of the antibodies or antigen-binding fragments described herein or known in the art); (ii) a buffer (e.g., any of the exemplary buffers described herein or known in the art); (iii) a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate; (iv) a stabilizer (e.g., any of the exemplary stabilizers described herein or known in the art); (v) a surfactant (e.g., any of the exemplary surfactants described herein or known in the art); and (vi) sterile water, where (i) to (vi) are mixed or combined in amounts sufficient to generate any of the formulations described herein.

Also provided herein are methods of making an aqueous antibody formulation that include mixing or combining: (i) an antibody or an antigen-binding fragment thereof (e.g., any of the exemplary antibodies or antigen-binding fragments described herein or known in the art); and (ii) a salt selected from the group of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, (iii) a stabilizer (e.g., any of the exemplary stabilizers described herein or known in the art); (iv) a surfactant (e.g., any of the exemplary surfactants described herein or known in the art); and (v) sterile water; wherein (i) to (v) are mixed or combined in amounts sufficient to generate any of the formulations described herein. In some embodiments of these methods, the method does not include mixing or combining a buffer with (i) and (ii), and the method results in a buffer-free aqueous antibody formulation.

Also provided are methods of treating a subject in need thereof that include administering to the subject a therapeutically effective amount of any of the formulations described herein.

Provided herein are aqueous antibody formulations that include: about 0.1 mg/mL to about 500 mg/mL of an antibody or an antigen-binding fragment thereof; about 1 mM to about 100 mM of a buffer; and about 1 mM to about 750 mM of a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, wherein the formulation has a pH of about 4 to about 8 and optionally, an osmolality of about 250 mOsm/kg to about 1500 mOsm/kg.

Also provided herein are aqueous antibody formulations that include: about 0.1 mg/mL to about 500 mg/mL of an antibody or an antigen-binding fragment thereof; and about 1 mM to about 750 mM of a salt selected from the group of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, where the formulation has a pH of about 4 to about 8. In some embodiments of any of the aqueous antibody formulations described herein, the formulation is a buffer-free aqueous antibody formulation.

In some embodiments of any of the aqueous antibody formulations described herein, the salt is magnesium glutamate, magnesium acetate, magnesium aspartate, or magnesium sulfate, or a combination thereof. In some embodiments of any of the aqueous antibody formulations described herein, the salt is magnesium glutamate. In some embodiments of any of the aqueous antibody formulations described herein, the salt is magnesium acetate. In some embodiments of any of the aqueous antibody formulations described herein, the salt is magnesium aspartate. In some embodiments of any of the aqueous antibody formulations described herein, the salt is magnesium sulfate. In some embodiments of any of the aqueous antibody formulations described herein, the formulation includes about 10 mM to about 750 mM of the salt. In some embodiments of any of the aqueous antibody formulations described herein, the formulation includes about 20 mM to about 750 mM of the salt.

In some embodiments of any of the aqueous antibody formulations described herein, the salt is sodium acetate, sodium aspartate, sodium glutamate or sodium sulfate. In some embodiments of any of the aqueous antibody formulations described herein, the salt is sodium acetate. In some embodiments of any of the aqueous antibody formulations described herein, the salt is sodium aspartate. In some embodiments of any of the aqueous antibody formulations described herein, the salt is sodium glutamate. In some embodiments of any of the aqueous antibody formulations described herein, the salt is sodium sulfate. In some embodiments of any of the aqueous antibody formulations described herein, the formulation includes about 10 mM to about 750 mM of the salt. In some embodiments of any of the aqueous antibody formulations described herein, the formulation includes about 20 mM to about 750 mM of the salt.

In some embodiments of any of the aqueous antibody formulations described herein, the salt is lithium acetate, lithium aspartate, lithium glutamate, or lithium sulfate. In some embodiments of any of the aqueous antibody formulations described herein, the salt is lithium acetate. In some embodiments of any of the aqueous antibody formulations described herein, the salt is lithium aspartate. In some embodiments of any of the aqueous antibody formulations described herein, the salt is lithium glutamate. In some embodiments of any of the aqueous antibody formulations described herein, the salt is lithium sulfate. In some embodiments of any of the aqueous antibody formulations described herein, the formulation includes about 10 mM to about 750 mM of the salt. In some embodiments of any of the aqueous antibody formulations described herein, the formulation includes about 20 mM to about 750 mM of the salt.

In some embodiments of any of the aqueous antibody formulations described herein, the buffer is selected from the group consisting of: acetate, succinate, gluconate, histidine, citrate, and phosphate. In some embodiments of any of the aqueous antibody formulations described herein, the buffer is a histidine buffer. In some embodiments of any of the aqueous antibody formulations described herein, the buffer is an acetate buffer. In some embodiments of any of the aqueous antibody formulations described herein, the buffer is a citrate buffer. In some embodiments of any of the aqueous antibody formulations described herein, the buffer is a phosphate buffer. In some embodiments of any of the aqueous antibody formulations described herein, the formulation includes about 1 mM to about 100 mM of the buffer. In some embodiments of any of the aqueous antibody formulations described herein, the formulation includes about 1 mM to about 75 mM of the buffer. In some embodiments of any of the aqueous antibody formulations described herein, the formulation includes about 1 mM to about 50 mM of the buffer. In some embodiments of any of the aqueous antibody formulations described herein, the formulation includes about 1 mM to about 20 mM of the buffer.

In some embodiments of any of the aqueous antibody formulations described herein, the formulation has a pH of about 5 to about 6. In some embodiments of any of the aqueous antibody formulations described herein, the formulation has a pH of about 5.5.

In some embodiments of any of the aqueous antibody formulations described herein, the formulation includes an antibody. In some embodiments of any of the aqueous antibody formulations described herein, the antibody is a monoclonal antibody (mAb). In some embodiments of any of the aqueous antibody formulations described herein, the mAb is a human antibody or a humanized antibody. In some embodiments of any of the aqueous antibody formulations described herein, the mAb has an Fc amino acid substitution that decreases its conformational stability as compared to a similar antibody not including the Fc amino acid substitution. In some embodiments of any of the aqueous antibody formulations described herein, the mAb is an IgG1 or an IgG4 antibody.

In some embodiments of any of the aqueous antibody formulations described herein, the mAb is an anti-C—X—C motif chemokine receptor 3 (CXCR3) mAb. In some embodiments of any of the aqueous antibody formulations described herein, the anti-CXCR3 mAb includes a heavy chain including SEQ ID NO: 1 and a light chain including SEQ ID NO: 2.

In some embodiments of any of the aqueous antibody formulations described herein, the mAb is an anti-cluster of differentiation 38 (CD38)-Fc engineered mAb. In some embodiments of any of the aqueous antibody formulations described herein, the anti-CD38-Fc engineered mAb includes a heavy chain including SEQ ID NO: 3 and a light chain including SEQ ID NO: 4.

In some embodiments of any of the aqueous antibody formulations described herein, the monoclonal antibody is an anti-carcinoembryonic antigen-related cell adhesion molecule 5 (CEACAM5) monoclonal antibody. In some embodiments of any of the aqueous antibody formulations described herein, the anti-CEACAM5 monoclonal antibody comprises a heavy chain comprising SEQ ID NO: 7 and a light chain comprising SEQ ID NO: 8.

In some embodiments of any of the aqueous antibody formulations described herein, the monoclonal antibody is an anti-carcinoembryonic antigen-related cell adhesion molecule 5 (CEACAM5)-Fc engineered monoclonal antibody. In some embodiments of any of the aqueous antibody formulations described herein, the anti-CEACAM5-Fc engineered monoclonal antibody comprises a heavy chain comprising SEQ ID NO: 9 and a light chain comprising SEQ ID NO: 10. In some embodiments of any of the aqueous antibody formulations described herein, the anti-CEACAM5-Fc engineered monoclonal antibody comprises a heavy chain comprising SEQ ID NO: 11 and a light chain comprising SEQ ID NO: 12. In some embodiments of any of the aqueous antibody formulations described herein, the anti-CEACAM5-Fc engineered monoclonal antibody comprises a heavy chain comprising SEQ ID NO: 13 and a light chain comprising SEQ ID NO: 14.

In some embodiments of any of the aqueous antibody formulations described herein, the formulation includes about 0.1 mg/mL to 400 mg/mL of the antibody or the antigen-binding antibody fragment. In some embodiments of any of the aqueous antibody formulations described herein, the formulation includes about 0.1 mg/mL to 250 mg/mL of the antibody or the antigen-binding antibody fragment. In some embodiments of any of the aqueous antibody formulations described herein, the formulation includes about 0.1 mg/mL to about 200 mg/mL of the antibody or the antigen-binding antibody fragment. In some embodiments of any of the aqueous antibody formulations described herein, the formulation includes about 0.1 mg/mL to about 150 mg/mL of the antibody or the antigen-binding antibody fragment. In some embodiments of any of the aqueous antibody formulations described herein, the formulation includes about 0.1 mg/mL to about 100 mg/mL of the antibody or the antigen-binding antibody fragment. In some embodiments of any of the aqueous antibody formulations described herein, the formulation includes about 0.1 mg/mL to about 50 mg/mL of the antibody or the antigen-binding antibody fragment. In some embodiments of any of the aqueous antibody formulations described herein, the formulation includes about 0.1 mg/mL to about 25 mg/mL of the antibody or the antigen-binding antibody fragment.

In some embodiments of any of the aqueous antibody formulations described herein, the formulation has a viscosity of about 1 cP to about 50 cP. In some embodiments of any of the aqueous antibody formulations described herein, the formulation has a viscosity of about 1 cP to about 40 cP. In some embodiments of any of the aqueous antibody formulations described herein, the formulation has a viscosity of about 1 cP to about 30 cP. In some embodiments of any of the aqueous antibody formulations described herein, the formulation has a viscosity of about 1 cP to about 20 cP.

In some embodiments of any of the aqueous antibody formulations described herein, the formulation has an osmolality of about 250 mOsm/kg to about 1500 mOsm/kg. In some embodiments of any of the aqueous antibody formulations described herein, the formulation has an osmolality of about 250 mOsm/kg to about 1500 mOsm/kg. In some embodiments of any of the aqueous antibody formulations described herein, the formulation has an osmolality of about 250 mOsm/kg to about 750 mOsm/kg. In some embodiments of any of the aqueous antibody formulations described herein, the formulation has an osmolality of about 250 mOsm/kg to about 500 mOsm/kg. In some embodiments of any of the aqueous antibody formulations described herein, the formulation has an osmolality of about 250 mOsm/kg to about 400 mOsm/kg. In some embodiments of any of the aqueous antibody formulations described herein, the formulation has an osmolality of about 500 mOsm/kg to about 1500 mOsm/kg. In some embodiments of any of the aqueous antibody formulations described herein, the formulation has an osmolality of about 500 mOsm/kg to about 1000 mOsm/kg. In some embodiments of any of the aqueous antibody formulations described herein, the formulation has an osmolality of about 1000 mOsm/kg to about 1500 mOsm/kg.

In some embodiments of any of the aqueous antibody formulations described herein, the formulation is stable (e.g., % of high molecular weight (HMW) by SEC ≤5%) at 25° C. for about 1 week to about 2 years. In some embodiments of any of the aqueous antibody formulations described herein, the formulation is stable (e.g., % HMW by SEC ≤5%) at 40° C. for about 1 hour to about 8 weeks.

In some embodiments of any of the aqueous antibody formulations described herein, the formulation is suitable for intravenous, intramuscular, or subcutaneous administration. In some embodiments of any of the aqueous antibody formulations described herein, the formulation is suitable for intravenous administration. In some embodiments of any of the aqueous antibody formulations described herein, the formulation is suitable for subcutaneous administration.

Some embodiments of any of the aqueous antibody formulations described herein further includes one or more of a stabilizer, an anti-oxidant, a metal chelator, a viscosity modifier, an amino acid, and a surfactant. In some embodiments of any of the aqueous antibody formulations described herein, the stabilizer is fructose, maltose, galactose, glucose, O-mannose, sorbose, lactose, sucrose, trehalose, cellobiose, raffinose, melezitose, a maltodextrin, a dextran, starch, mannitol, xylitol, maltitol, lactitol, glucitol, sucrose, trehalose, raffinose, maltose, sorbitol, mannitol, an amino sugar, sodium chloride, and glycerol.

In some embodiments of any of the aqueous antibody formulations described herein, the antioxidant is methionine, ascorbic acid, or N-acetyl cysteine. In some embodiments of any of the aqueous antibody formulations described herein, the metal chelator is sodium ethylenediaminetetraacetic acid (EDTA), calcium EDTA, or diethylenetriamine pentaacetate (DTPA). In some embodiments of any of the aqueous antibody formulations described herein, the viscosity modifier is arginine, histidine, lysine, proline, glycine, or sodium chloride.

In some embodiments of any of the aqueous antibody formulations described herein, the surfactant is selected from the group of: sorbitan monocaprylate, sorbitan monolaurate, sorbitan monopalmitate, sorbitan trioleate, glycerine monocaprylate, glycerine monomyristate, glycerine monostearate, decaglyceryl monostearate, decaglyceryl distearate, decaglyceryl monolinoleate, polyoxyethylene sorbitan monolaurate, polyoxythylene sorbitan monocleate, polyoxyethylene sorbitan monostearate, polyoxyethylene sorbitan monopalmitate, polyoxyethyene sorbitan trioleate, polyoxyethylene sorbitan tristearate, polyoxyethylene sorbitol tetrastearate, polyoxyethylene sorbitol tetraoleate, polyoxyethylene glyceryl monostearate, polyethylene glycol distearate, polyoxyethylene lauryl ether, polyoxyethylene polyoxypropylene glycol, polyoxyethylene polyoxypropylene propyl ether, polyoxyethylene polyoxypropylene cetyl ether, polyoxyethylene nonylphenyl ether, polyoxythylene castor oil, polyoxyethylene hydrogenated castor oil, polyoxyethylene sorbitol beeswax, polyoxyethylene lanolin, polyoxyethylene stearic acid amide, sodium cetyl sulfate, sodium lauryl sulfate, sodium oleyl sulfate, sodium polyoxyethylene lauryl sulfate, sodium lauryl sulfosuccinate ester, lecithin, a glycerophosopholipid, a sphingophospholipid, polysorbate 20, polysorbate 40, polysorbate 60, polysorbate 80, poloxamer 188, triton-X, sodium lauryl sulfate, polyethylene glycol, and propylene glycol.

In some embodiments of any of the aqueous antibody formulations described herein, the amino acid is selected from the group of: arginine, lysine, histidine, proline, ornithine, isoleucine, leucine, alanine, glycine, glutamic acid, and aspartic acid.

Also provided herein are injection devices including any of the aqueous antibody formulations described herein.

Also provided herein are kits including one or more vials containing any of the aqueous antibody formulations described herein. In some embodiments of any of the kits described herein, the kit further includes an injection device for administration of the aqueous antibody formulation to a subject in need thereof.

Provided herein are methods of making an aqueous antibody formulation that include mixing or combining: (i) an antibody or an antigen-binding fragment thereof; (ii) a buffer; (iii) a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, (iv) a stabilizer; (v) a surfactant; and (vi) sterile water, wherein (i) to (vi) are mixed or combined in amounts sufficient to generate any of the aqueous antibody formulations described herein.

Also provided herein are methods of making an aqueous antibody formulation that include mixing or combining: (i) an antibody or an antigen-binding fragment thereof; and (ii) a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate; (iii) a stabilizer); (iv) a surfactant); and (v) sterile water, wherein (i) to (v) are mixed or combined in amounts sufficient to generate any of the aqueous antibody formulations described herein. In some embodiments of any of the aqueous antibody formulations described herein, the method does not include mixing a buffer with (i) to (v) and the method results in the generation of a buffer-free aqueous antibody formulation.

Some embodiments of any of the methods described herein further include mixing or combining one or more (e.g., one, two or three) of an antioxidant, a metal chelator, and a viscosity modifier to (i) and (vi).

Also provided herein are methods of treating a subject in need thereof, the method includes administering to the subject a therapeutically effective amount of any of the aqueous antibody formulations described herein.

The term “stabilizer” refers to an additional agent (e.g., not including any of the salts of magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate) that improves or otherwise enhances stability of a protein (e.g., an antibody or an antigen-binding antibody fragment) in a formulation. Non-limiting examples of stabilizers are described herein. Additional examples of stabilizers are known in the art. The term “surfactant” generally includes an agent that protects a protein (e.g., an antibody or an antigen-binding antibody fragment) from air/solution interface-induced stress and/or solution/surface induced-stress. In some embodiments, a surfactant may protect a protein (e.g., an antibody or an antigen-binding antibody fragment) from aggregation. Non-limiting examples of surfactants are described herein. Additional examples of surfactants are known in the art.

The term “subject” refers to any mammal. In some embodiments, the subject or “subject in need of treatment” can be a canine (e.g., a dog), feline (e.g., a cat), equine (e.g., a horse), ovine, bovine, porcine, caprine, primate, e.g., a simian (e.g., a monkey (e.g., a marmoset, baboon), or an ape (e.g., a gorilla, chimpanzee, orangutan, or gibbon), a human; or a rodent (e.g., a mouse, a guinea pig, a hamster, or a rat). In some embodiments, the subject or “subject suitable for treatment” may be a non-human mammal, especially mammals that are conventionally used as models for demonstrating therapeutic efficacy in humans (e.g., murine, lapine, porcine, canine or primate animals) may be employed.

As used herein, “treating” means a reduction in the number, severity, or frequency of one or more symptoms of a medical disease or condition in a subject (e.g., any of the exemplary subjects described herein).

As used herein, “buffer-free” means no or trace amount of a buffer (e.g., any of the buffers described herein).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing the lowest unfolding temperature (Tₘ₁) for antibody A (1 mg/mL) in histidine-buffered solutions (pH 5.5) containing labeled excipients at 200 mM concentration.

FIG. 2 is a graph showing k_{agg} for 150 mg/mL antibody A following storage at 40° C. vs. increase in Tₘ₁ (ΔTₘ₁) in histidine-buffered solutions (pH 5.5) containing labeled excipients at 200 mM concentration. Solution with no added excipient is labeled Control.

FIG. 3 is a graph showing ΔTₘ₁ for antibody B (1 mg/mL) in histidine-buffered solutions (pH 6.2) containing labeled excipients at 200 mM concentration.

FIG. 4 is a graph showing rates of aggregation (k_{agg}) for 50 mg/mL antibody B following storage at 40° C. vs. ΔTₘ₁ in histidine-buffered solutions (pH 6.2) containing labeled excipients at 200 mM concentration. Solution with no added excipient is labeled Control.

FIG. 5 is a graph showing the effect of excipient concentration on Tₘ₁ for antibody A in solution (1 mg/mL) in histidine-buffered solutions (pH 5.5) containing labeled excipients.

FIG. 6 is a graph showing the percent aggregate for 150 mg/mL antibody A following storage at 40° C. for 4 weeks in histidine-buffered solutions (pH 5.5) containing labeled excipients.

FIG. 7 is a graph showing the effect of excipient concentration on Tₘ₁ for antibody C (1 mg/mL) in histidine-buffered solutions (pH 6.2) containing labeled excipients.

FIG. 8 is a graph showing increase in Tₘ₁ for wild-type anti-CEACAM5 antibody and its antibody D, antibody E, and antibody DE mutants (1 mg/mL) in histidine-buffered (pH 5.5) solutions with labeled salts at 200 mM compared to the respective salt-free solutions.

FIG. 9 is a graph showing the viscosity of ˜150 mg/mL antibody C solutions at 20° C. in histidine-buffered solutions (pH 6.2) containing labeled excipients at 200 mM concentration. Solution with no added excipient is labeled Control.

FIG. 10 is a graph showing the viscosity of ˜150 mg/mL antibody A solutions at 20° C. vs. ΔTₘ₁ in histidine-buffered solutions (pH 5.5) containing labeled excipients at 200 mM concentration or sucrose between 2% and 30% (2% sucrose, 5% sucrose, 10% sucrose, 15% sucrose and 30% sucrose). Solution with no added excipient is labeled Control.

FIG. 11 is a graph showing the osmolality of ˜150 mg/mL antibody A solutions at 20° C. vs. ΔTₘ₁ in histidine-buffered solutions (pH 5.5) containing labeled excipients at 200 mM concentration or sucrose between 2% and 30% (2% sucrose, 5% sucrose, 10% sucrose, 15% sucrose and 30% sucrose).

### DETAILED DESCRIPTION

Over the past few decades, therapies involving the use of monoclonal antibodies and other Fc-derived antigen-binding proteins have become a mainstay of modern medicine. There is an ever-increasing reliance on these complex molecules in various therapeutic areas including but not limited to oncology, immunology, immuno-oncology, cardiology with nearly 100 molecules approved for therapeutic use to date and more than 500 at various stages of development or clinical trials.

A fundamental aspect for ensuring the transition of these therapeutic entities from the lab into manufacturable and marketable products of high and consistent quality is their stability in the dosage form. Owing to their complex chemistry and structure, proteins are susceptible to various forms of physical and chemical degradation that can compromise the biological efficacy and safety of the final drug product. Protein aggregation for example is a key quality attribute that is routinely monitored for protein-based products and is critical to the determination of product shelf life. At a fundamental level, protein aggregation is linked to the stability of the native form of the protein, with a growth in non-native cell (e.g., a non-native mammalian cell) generally linked to an increased rate and extent of aggregation. Thus, it is no surprise that attempts to control and minimize aggregation during product shelf life (kinetic stability) are often mediated through the use of excipients or formulation conditions intended to increase conformational stability of the protein. Essentially, the intent is to stabilize the protein in its native conformation in order to minimize the population of aggregation-competent “non-native” species. Sugars and polyols, such as sucrose, trehalose, mannitol, sorbitol etc. are often used to stabilize proteins in their native state and reduce rates of aggregation. However an unwanted effect of using these stabilizers is the concentration-dependent increase in solution viscosity.

Solution viscosity is a key attribute of protein products especially those that are formulated at high protein concentrations (for example ≥100 mg/mL for an antibody or Fc-derived proteins of similar molecular weight) and it can critically impact the utility and success of the product. The manufacturability of a product and the end use by the patient or healthcare practitioner is intimately linked to the ability of a solution to flow seamlessly. High viscosity, for example, can necessitate the use of specialized administration devices or protocols which may not always be suitable for the desired population thereby limiting the use of the product. In other instances, high solution viscosity may require the application of manufacturing technologies which may negatively impact the stability of the protein (for example high-temperature processing). It is thus not unusual to employ viscosity-reducing excipients, such as salts and amino acids, in high protein concentration solutions. However, these excipients can negatively impact the stability of the protein thereby resulting in solutions with an increased aggregation rate compared to high-viscosity control solutions lacking the viscosity-reducing agent. In essence, commonly employed stabilizers and the viscosity-reducing excipients can have an opposite effect on product performance thereby complicating its development.

Another critical attribute for injectable products (most protein-based products) that needs to be considered is its osmolality. While intravenous solutions generally need to be isotonic, it is not unusual for subcutaneous solutions to be hypertonic. In fact, there is evidence in literature of hypertonic formulations resulting in enhanced protein bioavailability following subcutaneous administration (Fathallah, A. M. et al, Biopharm Drug Dispos. 2015 March; 36(2):115-25). Thus, the impact of solution osmolality (and thus tonicity) on injection site discomfort and/or reaction as well as bioavailability in the patient population needs to be carefully monitored and characterized during clinical development phases.

Thus, there is a need in the art of formulating antibody and other Fc-derived products for developing stable, low-viscosity solution formulations with well characterized osmotic properties.

Provided herein are aqueous antibody formulations that include about 0.1 mg/mL to about 500 mg/mL of an antibody or an antigen-binding fragment thereof (e.g., any of the exemplary antibodies or antigen-binding fragments described herein or known in the art); about 1 mM to about 100 mM of a buffer (e.g., any of the exemplary buffers described herein or known in the art); and about 1 mM to 750 mM of a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, where the formulations have a pH of about 4 to about 8.

Also provided herein are aqueous antibody formulations include about 0.1 mg/mL to about 500 mg/mL (e.g., any of the subranges of this range described herein) of an antibody or an antigen-binding fragment thereof (e.g., any of the exemplary antibodies or antigen-binding fragments described herein or known in the art); and about 1 mM to 750 mM (e.g., any of the subranges of this range described herein) of a salt selected from the group of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, where the formulations have a pH of about 4 to about 8. In some embodiments, the aqueous antibody formulation is a buffer-free aqueous antibody formulation.

Also provided are injection devices that include any of these formulations, and kits including one or more vials containing any of these formulations.

Also provided are methods of making an aqueous antibody formulation that include mixing or combining: (i) an antibody or an antigen-binding fragment thereof (e.g., any of the antibodies or antigen-binding fragments described herein or known in the art); (ii) a buffer (e.g., any of the exemplary buffers described herein or known in the art); (iii) a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, (iv) a stabilizer; (v) a surfactant; and (vi) sterile water, where (i) to (vi) are mixed or combined in amounts sufficient to generate any of the formulations described herein.

Also provided herein are methods of making an aqueous antibody formulations that include mixing or combining: (i) an antibody or an antigen-binding fragment thereof; and (ii) a salt selected from the group of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate; (iii) a stabilizer); (iv) a surfactant); and (v) sterile water, wherein (i) to (v) are mixed or combined in amounts sufficient to generate any of the aqueous antibody formulations described herein. In some embodiments of the methods described herein, the method does not include mixing or combining a buffer with (i) to (v) and the method results in a buffer-free aqueous antibody formulation.

Also provided are methods of treating a subject in need thereof that include administering to the subject a therapeutically effective amount of any of the formulations described herein.

Non-limiting aspects of these formulations, injection devices, kits, and methods are described below. As can be appreciated by those in the field, the exemplary aspects listed below can be used in any combination, and can be combined with other aspects known in the field.

### Antibodies and Antigen-Binding Antibody Fragments

The aqueous antibody formulations provided herein can include, e.g., about 0.1 mg/mL to about 500 mg/mL, about 0.1 mg/mL to about 480 mg/mL, about 0.1 mg/mL to about 460 mg/mL, about 0.1 mg/mL to about 440 mg/mL, about 0.1 mg/mL to about 420 mg/mL, about 0.1 mg/mL to about 400 mg/mL, about 0.1 mg/mL to about 380 mg/mL, about 0.1 mg/mL to about 360 mg/mL, about 0.1 mg/mL to about 340 mg/mL, about 0.1 mg/mL to about 320 mg/mL, about 0.1 mg/mL to about 300 mg/mL, about 0.1 mg/mL to about 280 mg/mL, about 0.1 mg/mL to about 260 mg/mL, about 0.1 mg/mL to about 240 mg/mL, about 0.1 mg/mL to about 220 mg/mL, about 0.1 mg/mL to about 200 mg/mL, about 0.1 mg/mL to about 190 mg/mL, about 0.1 mg/mL to about 180 mg/mL, about 0.1 mg/mL to about 170 mg/mL, about 0.1 mg/mL to about 160 mg/mL, about 0.1 mg/mL to about 140 mg/mL, about 0.1 mg/mL to about 130 mg/mL, about 0.1 mg/mL to about 120 mg/mL, about 0.1 mg/mL to about 110 mg/mL, about 0.1 mg/mL to about 100 mg/mL, about 0.1 mg/mL to about 90 mg/mL, about 0.1 mg/mL to about 80 mg/mL, about 0.1 mg/mL to about 70 mg/mL, about 0.1 mg/mL to about 60 mg/mL, about 0.1 mg/mL to about 50 mg/mL, about 0.1 mg/mL to about 45 mg/mL, about 0.1 mg/mL to about 40 mg/mL, about 0.1 mg/mL to about 35 mg/mL, about 0.1 mg/mL to about 30 mg/mL, about 0.1 mg/mL to about 25 mg/mL, about 0.1 mg/mL to about 20 mg/mL, about 0.1 mg/mL to about 15 mg/mL, about 0.1 mg/mL to about 10 mg/mL, about 0.1 mg/mL to about 5 mg/mL, about 0.1 mg/mL to about 2.5 mg/mL, about 0.1 mg/mL to about 1.0 mg/mL, about 0.1 mg/mL to about 0.5 mg/mL, about 0.5 mg/mL to about 500 mg/mL, about 0.5 mg/mL to about 480 mg/mL, about 0.5 mg/mL to about 460 mg/mL, about 0.5 mg/mL to about 440 mg/mL, about 0.5 mg/mL to about 420 mg/mL, about 0.1 mg/mL to about 400 mg/mL, about 0.1 mg/mL to about 380 mg/mL, about 0.5 mg/mL to about 360 mg/mL, about 0.5 mg/mL to about 340 mg/mL, about 0.5 mg/mL to about 320 mg/mL, about 0.5 mg/mL to about 300 mg/mL, about 0.5 mg/mL to about 280 mg/mL, about 0.5 mg/mL to about 260 mg/mL, about 0.5 mg/mL to about 240 mg/mL, about 0.5 mg/mL to about 220 mg/mL, about 0.5 mg/mL to about 200 mg/mL, about 0.5 mg/mL to about 190 mg/mL, about 0.5 mg/mL to about 180 mg/mL, about 0.5 mg/mL to about 170 mg/mL, about 0.5 mg/mL to about 160 mg/mL, about 0.5 mg/mL to about 140 mg/mL, about 0.5 mg/mL to about 130 mg/mL, about 0.5 mg/mL to about 120 mg/mL, about 0.5 mg/mL to about 110 mg/mL, about 0.5 mg/mL to about 100 mg/mL, about 0.5 mg/mL to about 90 mg/mL, about 0.5 mg/mL to about 80 mg/mL, about 0.5 mg/mL to about 70 mg/mL, about 0.5 mg/mL to about 60 mg/mL, about 0.5 mg/mL to about 50 mg/mL, about 0.5 mg/mL to about 45 mg/mL, about 0.5 mg/mL to about 40 mg/mL, about 0.5 mg/mL to about 35 mg/mL, about 0.5 mg/mL to about 30 mg/mL, about 0.5 mg/mL to about 25 mg/mL, about 0.5 mg/mL to about 20 mg/mL, about 0.5 mg/mL to about 15 mg/mL, about 0.5 mg/mL to about 10 mg/mL, about 0.5 mg/mL to about 5 mg/mL, about 0.5 mg/mL to about 2.5 mg/mL, about 0.5 mg/mL to about 1.0 mg/mL, about 1.0 mg/mL to about 500 mg/mL, about 1.0 mg/mL to about 480 mg/mL, about 1.0 mg/mL to about 460 mg/mL, about 1.0 mg/mL to about 440 mg/mL, about 1.0 mg/mL to about 420 mg/mL, about 1.0 mg/mL to about 400 mg/mL, about 1.0 mg/mL to about 380 mg/mL, about 1.0 mg/mL to about 360 mg/mL, about 1.0 mg/mL to about 340 mg/mL, about 1.0 mg/mL to about 320 mg/mL, about 1.0 mg/mL to about 300 mg/mL, about 1.0 mg/mL to about 280 mg/mL, about 1.0 mg/mL to about 260 mg/mL, about 1.0 mg/mL to about 240 mg/mL, about 1.0 mg/mL to about 220 mg/mL, about 1.0 mg/mL to about 200 mg/mL, about 1.0 mg/mL to about 190 mg/mL, about 1.0 mg/mL to about 180 mg/mL, about 1.0 mg/mL to about 170 mg/mL, about 1.0 mg/mL to about 160 mg/mL, about 1.0 mg/mL to about 140 mg/mL, about 1.0 mg/mL to about 130 mg/mL, about 1.0 mg/mL to about 120 mg/mL, about 1.0 mg/mL to about 110 mg/mL, about 1.0 mg/mL to about 100 mg/mL, about 1.0 mg/mL to about 90 mg/mL, about 1.0 mg/mL to about 80 mg/mL, about 1.0 mg/mL to about 70 mg/mL, about 1.0 mg/mL to about 60 mg/mL, about 1.0 mg/mL to about 50 mg/mL, about 1.0 mg/mL to about 45 mg/mL, about 1.0 mg/mL to about 40 mg/mL, about 1.0 mg/mL to about 35 mg/mL, about 1.0 mg/mL to about 30 mg/mL, about 1.0 mg/mL to about 25 mg/mL, about 1.0 mg/mL to about 20 mg/mL, about 1.0 mg/mL to about 15 mg/mL, about 1.0 mg/mL to about 10 mg/mL, about 1.0 mg/mL to about 5 mg/mL, about 1.0 mg/mL to about 2.5 mg/mL, about 2.5 mg/mL to about 500 mg/mL, about 2.5 mg/mL to about 480 mg/mL, about 2.5 mg/mL to about 460 mg/mL, about 2.5 mg/mL to about 440 mg/mL, about 2.5 mg/mL to about 420 mg/mL, about 2.5 mg/mL to about 400 mg/mL, about 2.5 mg/mL to about 380 mg/mL, about 2.5 mg/mL to about 360 mg/mL, about 2.5 mg/mL to about 340 mg/mL, about 2.5 mg/mL to about 320 mg/mL, about 2.5 mg/mL to about 300 mg/mL, about 2.5 mg/mL to about 280 mg/mL, about 2.5 mg/mL to about 260 mg/mL, about 2.5 mg/mL to about 240 mg/mL, about 2.5 mg/mL to about 220 mg/mL, about 2.5 mg/mL to about 200 mg/mL, about 2.5 mg/mL to about 190 mg/mL, about 2.5 mg/mL to about 180 mg/mL, about 2.5 mg/mL to about 170 mg/mL, about 2.5 mg/mL to about 160 mg/mL, about 2.5 mg/mL to about 140 mg/mL, about 2.5 mg/mL to about 130 mg/mL, about 2.5 mg/mL to about 120 mg/mL, about 2.5 mg/mL to about 110 mg/mL, about 2.5 mg/mL to about 100 mg/mL, about 2.5 mg/mL to about 90 mg/mL, about 2.5 mg/mL to about 80 mg/mL, about 2.5 mg/mL to about 70 mg/mL, about 2.5 mg/mL to about 60 mg/mL, about 2.5 mg/mL to about 50 mg/mL, about 2.5 mg/mL to about 45 mg/mL, about 2.5 mg/mL to about 40 mg/mL, about 2.5 mg/mL to about 35 mg/mL, about 2.5 mg/mL to about 30 mg/mL, about 2.5 mg/mL to about 25 mg/mL, about 2.5 mg/mL to about 20 mg/mL, about 2.5 mg/mL to about 15 mg/mL, about 2.5 mg/mL to about 10 mg/mL, about 2.5 mg/mL to about 5 mg/mL, about 5 mg/mL to about 500 mg/mL, about 5 mg/mL to about 480 mg/mL, about 5 mg/mL to about 460 mg/mL, about 5 mg/mL to about 440 mg/mL, about 5 mg/mL to about 420 mg/mL, about 5 mg/mL to about 400 mg/mL, about 5 mg/mL to about 380 mg/mL, about 5 mg/mL to about 360 mg/mL, about 5 mg/mL to about 340 mg/mL, about 5 mg/mL to about 320 mg/mL, about 5 mg/mL to about 300 mg/mL, about 5 mg/mL to about 280 mg/mL, about 5 mg/mL to about 260 mg/mL, about 5 mg/mL to about 240 mg/mL, about 5 mg/mL to about 220 mg/mL, about 5 mg/mL to about 200 mg/mL, about 5 mg/mL to about 190 mg/mL, about 5 mg/mL to about 180 mg/mL, about 5 mg/mL to about 170 mg/mL, about 5 mg/mL to about 160 mg/mL, about 5 mg/mL to about 140 mg/mL, about 5 mg/mL to about 130 mg/mL, about 5 mg/mL to about 120 mg/mL, about 5 mg/mL to about 110 mg/mL, about 5 mg/mL to about 100 mg/mL, about 5 mg/mL to about 90 mg/mL, about 5 mg/mL to about 80 mg/mL, about 5 mg/mL to about 70 mg/mL, about 5 mg/mL to about 60 mg/mL, about 5 mg/mL to about 50 mg/mL, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 5 mg/mL to about 10 mg/mL, about 10 mg/mL to about 500 mg/mL, about 10 mg/mL to about 480 mg/mL, about 10 mg/mL to about 460 mg/mL, about 10 mg/mL to about 440 mg/mL, about 10 mg/mL to about 420 mg/mL, about 10 mg/mL to about 400 mg/mL, about 10 mg/mL to about 380 mg/mL, about 10 mg/mL to about 360 mg/mL, about 10 mg/mL to about 340 mg/mL, about 10 mg/mL to about 320 mg/mL, about 10 mg/mL to about 300 mg/mL, about 10 mg/mL to about 280 mg/mL, about 10 mg/mL to about 260 mg/mL, about 10 mg/mL to about 240 mg/mL, about 10 mg/mL to about 220 mg/mL, about 10 mg/mL to about 200 mg/mL, about 10 mg/mL to about 190 mg/mL, about 10 mg/mL to about 180 mg/mL, about 10 mg/mL to about 170 mg/mL, about 10 mg/mL to about 160 mg/mL, about 10 mg/mL to about 140 mg/mL, about 10 mg/mL to about 130 mg/mL, about 10 mg/mL to about 120 mg/mL, about 10 mg/mL to about 110 mg/mL, about 10 mg/mL to about 100 mg/mL, about 10 mg/mL to about 90 mg/mL, about 10 mg/mL to about 80 mg/mL, about 10 mg/mL to about 70 mg/mL, about 10 mg/mL to about 60 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 500 mg/mL, about 15 mg/mL to about 480 mg/mL, about 15 mg/mL to about 460 mg/mL, about 15 mg/mL to about 440 mg/mL, about 15 mg/mL to about 420 mg/mL, about 15 mg/mL to about 400 mg/mL, about 15 mg/mL to about 380 mg/mL, about 15 mg/mL to about 360 mg/mL, about 15 mg/mL to about 340 mg/mL, about 15 mg/mL to about 320 mg/mL, about 15 mg/mL to about 300 mg/mL, about 15 mg/mL to about 280 mg/mL, about 15 mg/mL to about 260 mg/mL, about 15 mg/mL to about 240 mg/mL, about 15 mg/mL to about 220 mg/mL, about 15 mg/mL to about 200 mg/mL, about 15 mg/mL to about 190 mg/mL, about 15 mg/mL to about 180 mg/mL, about 15 mg/mL to about 170 mg/mL, about 15 mg/mL to about 160 mg/mL, about 15 mg/mL to about 140 mg/mL, about 15 mg/mL to about 130 mg/mL, about 15 mg/mL to about 120 mg/mL, about 15 mg/mL to about 110 mg/mL, about 15 mg/mL to about 100 mg/mL, about 15 mg/mL to about 90 mg/mL, about 15 mg/mL to about 80 mg/mL, about 15 mg/mL to about 70 mg/mL, about 15 mg/mL to about 60 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 500 mg/mL, about 20 mg/mL to about 480 mg/mL, about 20 mg/mL to about 460 mg/mL, about 20 mg/mL to about 440 mg/mL, about 20 mg/mL to about 420 mg/mL, about 20 mg/mL to about 400 mg/mL, about 20 mg/mL to about 380 mg/mL, about 20 mg/mL to about 360 mg/mL, about 20 mg/mL to about 340 mg/mL, about 20 mg/mL to about 320 mg/mL, about 20 mg/mL to about 300 mg/mL, about 20 mg/mL to about 280 mg/mL, about 20 mg/mL to about 260 mg/mL, about 20 mg/mL to about 240 mg/mL, about 20 mg/mL to about 220 mg/mL, about 20 mg/mL to about 200 mg/mL, about 20 mg/mL to about 190 mg/mL, about 20 mg/mL to about 180 mg/mL, about 20 mg/mL to about 170 mg/mL, about 20 mg/mL to about 160 mg/mL, about 20 mg/mL to about 140 mg/mL, about 20 mg/mL to about 130 mg/mL, about 20 mg/mL to about 120 mg/mL, about 20 mg/mL to about 110 mg/mL, about 20 mg/mL to about 100 mg/mL, about 20 mg/mL to about 90 mg/mL, about 20 mg/mL to about 80 mg/mL, about 20 mg/mL to about 70 mg/mL, about 20 mg/mL to about 60 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 500 mg/mL, about 25 mg/mL to about 480 mg/mL, about 25 mg/mL to about 460 mg/mL, about 25 mg/mL to about 440 mg/mL, about 25 mg/mL to about 420 mg/mL, about 25 mg/mL to about 400 mg/mL, about 25 mg/mL to about 380 mg/mL, about 25 mg/mL to about 360 mg/mL, about 25 mg/mL to about 340 mg/mL, about 25 mg/mL to about 320 mg/mL, about 25 mg/mL to about 300 mg/mL, about 25 mg/mL to about 280 mg/mL, about 25 mg/mL to about 260 mg/mL, about 25 mg/mL to about 240 mg/mL, about 25 mg/mL to about 220 mg/mL, about 25 mg/mL to about 200 mg/mL, about 25 mg/mL to about 190 mg/mL, about 25 mg/mL to about 180 mg/mL, about 25 mg/mL to about 170 mg/mL, about 25 mg/mL to about 160 mg/mL, about 25 mg/mL to about 140 mg/mL, about 25 mg/mL to about 130 mg/mL, about 25 mg/mL to about 120 mg/mL, about 25 mg/mL to about 110 mg/mL, about 25 mg/mL to about 100 mg/mL, about 25 mg/mL to about 90 mg/mL, about 25 mg/mL to about 80 mg/mL, about 25 mg/mL to about 70 mg/mL, about 25 mg/mL to about 60 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 30 mg/mL to about 500 mg/mL, about 30 mg/mL to about 480 mg/mL, about 30 mg/mL to about 460 mg/mL, about 30 mg/mL to about 440 mg/mL, about 30 mg/mL to about 420 mg/mL, about 30 mg/mL to about 400 mg/mL, about 30 mg/mL to about 380 mg/mL, about 30 mg/mL to about 360 mg/mL, about 30 mg/mL to about 340 mg/mL, about 30 mg/mL to about 320 mg/mL, about 30 mg/mL to about 300 mg/mL, about 30 mg/mL to about 280 mg/mL, about 30 mg/mL to about 260 mg/mL, about 30 mg/mL to about 240 mg/mL, about 30 mg/mL to about 220 mg/mL, about 30 mg/mL to about 200 mg/mL, about 30 mg/mL to about 190 mg/mL, about 30 mg/mL to about 180 mg/mL, about 30 mg/mL to about 170 mg/mL, about 30 mg/mL to about 160 mg/mL, about 30 mg/mL to about 140 mg/mL, about 30 mg/mL to about 130 mg/mL, about 30 mg/mL to about 120 mg/mL, about 30 mg/mL to about 110 mg/mL, about 30 mg/mL to about 100 mg/mL, about 30 mg/mL to about 90 mg/mL, about 30 mg/mL to about 80 mg/mL, about 30 mg/mL to about 70 mg/mL, about 30 mg/mL to about 60 mg/mL, about 30 mg/mL to about 50 mg/mL, about 30 mg/mL to about 45 mg/mL, about 30 mg/mL to about 40 mg/mL, about 30 mg/mL to about 35 mg/mL, about 35 mg/mL to about 500 mg/mL, about 35 mg/mL to about 480 mg/mL, about 35 mg/mL to about 460 mg/mL, about 35 mg/mL to about 440 mg/mL, about 35 mg/mL to about 420 mg/mL, about 35 mg/mL to about 400 mg/mL, about 35 mg/mL to about 380 mg/mL, about 35 mg/mL to about 360 mg/mL, about 35 mg/mL to about 340 mg/mL, about 35 mg/mL to about 320 mg/mL, about 35 mg/mL to about 300 mg/mL, about 35 mg/mL to about 280 mg/mL, about 35 mg/mL to about 260 mg/mL, about 35 mg/mL to about 240 mg/mL, about 35 mg/mL to about 220 mg/mL, about 35 mg/mL to about 200 mg/mL, about 35 mg/mL to about 190 mg/mL, about 35 mg/mL to about 180 mg/mL, about 35 mg/mL to about 170 mg/mL, about 35 mg/mL to about 160 mg/mL, about 35 mg/mL to about 140 mg/mL, about 35 mg/mL to about 130 mg/mL, about 35 mg/mL to about 120 mg/mL, about 35 mg/mL to about 110 mg/mL, about 35 mg/mL to about 100 mg/mL, about 35 mg/mL to about 90 mg/mL, about 35 mg/mL to about 80 mg/mL, about 35 mg/mL to about 70 mg/mL, about 35 mg/mL to about 60 mg/mL, about 35 mg/mL to about 50 mg/mL, about 35 mg/mL to about 45 mg/mL, about 35 mg/mL to about 40 mg/mL, about 40 mg/mL to about 500 mg/mL, about 40 mg/mL to about 480 mg/mL, about 40 mg/mL to about 460 mg/mL, about 40 mg/mL to about 440 mg/mL, about 40 mg/mL to about 420 mg/mL, about 40 mg/mL to about 400 mg/mL, about 40 mg/mL to about 380 mg/mL, about 40 mg/mL to about 360 mg/mL, about 40 mg/mL to about 340 mg/mL, about 40 mg/mL to about 320 mg/mL, about 40 mg/mL to about 300 mg/mL, about 40 mg/mL to about 280 mg/mL, about 40 mg/mL to about 260 mg/mL, about 40 mg/mL to about 240 mg/mL, about 40 mg/mL to about 220 mg/mL, about 40 mg/mL to about 200 mg/mL, about 40 mg/mL to about 190 mg/mL, about 40 mg/mL to about 180 mg/mL, about 40 mg/mL to about 170 mg/mL, about 40 mg/mL to about 160 mg/mL, about 40 mg/mL to about 140 mg/mL, about 40 mg/mL to about 130 mg/mL, about 40 mg/mL to about 120 mg/mL, about 40 mg/mL to about 110 mg/mL, about 40 mg/mL to about 100 mg/mL, about 40 mg/mL to about 90 mg/mL, about 40 mg/mL to about 80 mg/mL, about 40 mg/mL to about 70 mg/mL, about 40 mg/mL to about 60 mg/mL, about 40 mg/mL to about 50 mg/mL, about 40 mg/mL to about 45 mg/mL, about 45 mg/mL to about 500 mg/mL, about 45 mg/mL to about 480 mg/mL, about 45 mg/mL to about 460 mg/mL, about 45 mg/mL to about 440 mg/mL, about 45 mg/mL to about 420 mg/mL, about 45 mg/mL to about 400 mg/mL, about 45 mg/mL to about 380 mg/mL, about 45 mg/mL to about 360 mg/mL, about 45 mg/mL to about 340 mg/mL, about 45 mg/mL to about 320 mg/mL, about 45 mg/mL to about 300 mg/mL, about 45 mg/mL to about 280 mg/mL, about 45 mg/mL to about 260 mg/mL, about 45 mg/mL to about 240 mg/mL, about 45 mg/mL to about 220 mg/mL, about 45 mg/mL to about 200 mg/mL, about 45 mg/mL to about 190 mg/mL, about 45 mg/mL to about 180 mg/mL, about 45 mg/mL to about 170 mg/mL, about 45 mg/mL to about 160 mg/mL, about 45 mg/mL to about 140 mg/mL, about 45 mg/mL to about 130 mg/mL, about 45 mg/mL to about 120 mg/mL, about 45 mg/mL to about 110 mg/mL, about 45 mg/mL to about 100 mg/mL, about 45 mg/mL to about 90 mg/mL, about 45 mg/mL to about 80 mg/mL, about 45 mg/mL to about 70 mg/mL, about 45 mg/mL to about 60 mg/mL, about 45 mg/mL to about 50 mg/mL, about 50 mg/mL to about 500 mg/mL, about 50 mg/mL to about 480 mg/mL, about 50 mg/mL to about 460 mg/mL, about 50 mg/mL to about 440 mg/mL, about 50 mg/mL to about 420 mg/mL, about 50 mg/mL to about 400 mg/mL, about 50 mg/mL to about 380 mg/mL, about 50 mg/mL to about 360 mg/mL, about 50 mg/mL to about 340 mg/mL, about 50 mg/mL to about 320 mg/mL, about 50 mg/mL to about 300 mg/mL, about 50 mg/mL to about 280 mg/mL, about 50 mg/mL to about 260 mg/mL, about 50 mg/mL to about 240 mg/mL, about 50 mg/mL to about 220 mg/mL, about 50 mg/mL to about 200 mg/mL, about 50 mg/mL to about 190 mg/mL, about 50 mg/mL to about 180 mg/mL, about 50 mg/mL to about 170 mg/mL, about 50 mg/mL to about 160 mg/mL, about 50 mg/mL to about 140 mg/mL, about 50 mg/mL to about 130 mg/mL, about 50 mg/mL to about 120 mg/mL, about 50 mg/mL to about 110 mg/mL, about 50 mg/mL to about 100 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 500 mg/mL, about 60 mg/mL to about 480 mg/mL, about 60 mg/mL to about 460 mg/mL, about 60 mg/mL to about 440 mg/mL, about 60 mg/mL to about 420 mg/mL, about 60 mg/mL to about 400 mg/mL, about 60 mg/mL to about 380 mg/mL, about 60 mg/mL to about 360 mg/mL, about 60 mg/mL to about 340 mg/mL, about 60 mg/mL to about 320 mg/mL, about 60 mg/mL to about 300 mg/mL, about 60 mg/mL to about 280 mg/mL, about 60 mg/mL to about 260 mg/mL, about 60 mg/mL to about 240 mg/mL, about 60 mg/mL to about 220 mg/mL, about 60 mg/mL to about 200 mg/mL, about 60 mg/mL to about 190 mg/mL, about 60 mg/mL to about 180 mg/mL, about 60 mg/mL to about 170 mg/mL, about 60 mg/mL to about 160 mg/mL, about 60 mg/mL to about 140 mg/mL, about 60 mg/mL to about 130 mg/mL, about 60 mg/mL to about 120 mg/mL, about 60 mg/mL to about 110 mg/mL, about 60 mg/mL to about 100 mg/mL, about 60 mg/mL to about 90 mg/mL, about 60 mg/mL to about 80 mg/mL, about 60 mg/mL to about 70 mg/mL, about 70 mg/mL to about 500 mg/mL, about 70 mg/mL to about 480 mg/mL, about 70 mg/mL to about 460 mg/mL, about 70 mg/mL to about 440 mg/mL, about 70 mg/mL to about 420 mg/mL, about 70 mg/mL to about 400 mg/mL, about 70 mg/mL to about 380 mg/mL, about 70 mg/mL to about 360 mg/mL, about 70 mg/mL to about 340 mg/mL, about 70 mg/mL to about 320 mg/mL, about 70 mg/mL to about 300 mg/mL, about 70 mg/mL to about 280 mg/mL, about 70 mg/mL to about 260 mg/mL, about 70 mg/mL to about 240 mg/mL, about 70 mg/mL to about 220 mg/mL, about 70 mg/mL to about 200 mg/mL, about 70 mg/mL to about 190 mg/mL, about 70 mg/mL to about 180 mg/mL, about 70 mg/mL to about 170 mg/mL, about 70 mg/mL to about 160 mg/mL, about 70 mg/mL to about 140 mg/mL, about 70 mg/mL to about 130 mg/mL, about 70 mg/mL to about 120 mg/mL, about 70 mg/mL to about 110 mg/mL, about 70 mg/mL to about 100 mg/mL, about 70 mg/mL to about 90 mg/mL, about 70 mg/mL to about 80 mg/mL, about 80 mg/mL to about 500 mg/mL, about 80 mg/mL to about 480 mg/mL, about 80 mg/mL to about 460 mg/mL, about 80 mg/mL to about 440 mg/mL, about 80 mg/mL to about 420 mg/mL, about 80 mg/mL to about 400 mg/mL, about 80 mg/mL to about 380 mg/mL, about 80 mg/mL to about 360 mg/mL, about 80 mg/mL to about 340 mg/mL, about 80 mg/mL to about 320 mg/mL, about 80 mg/mL to about 300 mg/mL, about 80 mg/mL to about 280 mg/mL, about 80 mg/mL to about 260 mg/mL, about 80 mg/mL to about 240 mg/mL, about 80 mg/mL to about 220 mg/mL, about 80 mg/mL to about 200 mg/mL, about 80 mg/mL to about 190 mg/mL, about 80 mg/mL to about 180 mg/mL, about 80 mg/mL to about 170 mg/mL, about 80 mg/mL to about 160 mg/mL, about 80 mg/mL to about 140 mg/mL, about 80 mg/mL to about 130 mg/mL, about 80 mg/mL to about 120 mg/mL, about 80 mg/mL to about 110 mg/mL, about 80 mg/mL to about 100 mg/mL, about 80 mg/mL to about 90 mg/mL about 90 mg/mL to about 500 mg/mL, about 90 mg/mL to about 480 mg/mL, about 90 mg/mL to about 460 mg/mL, about 90 mg/mL to about 440 mg/mL, about 90 mg/mL to about 420 mg/mL, about 90 mg/mL to about 400 mg/mL, about 90 mg/mL to about 380 mg/mL, about 90 mg/mL to about 360 mg/mL, about 90 mg/mL to about 340 mg/mL, about 90 mg/mL to about 320 mg/mL, about 90 mg/mL to about 300 mg/mL, about 90 mg/mL to about 280 mg/mL, about 90 mg/mL to about 260 mg/mL, about 90 mg/mL to about 240 mg/mL, about 90 mg/mL to about 220 mg/mL, about 90 mg/mL to about 200 mg/mL, about 90 mg/mL to about 190 mg/mL, about 90 mg/mL to about 180 mg/mL, about 90 mg/mL to about 170 mg/mL, about 90 mg/mL to about 160 mg/mL, about 90 mg/mL to about 140 mg/mL, about 90 mg/mL to about 130 mg/mL, about 90 mg/mL to about 120 mg/mL, about 90 mg/mL to about 110 mg/mL, about 90 mg/mL to about 100 mg/mL, about 100 mg/mL to about 500 mg/mL, about 100 mg/mL to about 480 mg/mL, about 100 mg/mL to about 460 mg/mL, about 100 mg/mL to about 440 mg/mL, about 100 mg/mL to about 420 mg/mL, about 100 mg/mL to about 400 mg/mL, about 100 mg/mL to about 380 mg/mL, about 100 mg/mL to about 360 mg/mL, about 100 mg/mL to about 340 mg/mL, about 100 mg/mL to about 320 mg/mL, about 100 mg/mL to about 300 mg/mL, about 100 mg/mL to about 280 mg/mL, about 100 mg/mL to about 260 mg/mL, about 100 mg/mL to about 240 mg/mL, about 100 mg/mL to about 220 mg/mL, about 100 mg/mL to about 200 mg/mL, about 100 mg/mL to about 190 mg/mL, about 100 mg/mL to about 180 mg/mL, about 100 mg/mL to about 170 mg/mL, about 100 mg/mL to about 160 mg/mL, about 100 mg/mL to about 140 mg/mL, about 100 mg/mL to about 130 mg/mL, about 100 mg/mL to about 120 mg/mL, about 100 mg/mL to about 110 mg/mL, about 110 mg/mL to about 500 mg/mL, about 110 mg/mL to about 480 mg/mL, about 110 mg/mL to about 460 mg/mL, about 110 mg/mL to about 440 mg/mL, about 110 mg/mL to about 420 mg/mL, about 110 mg/mL to about 400 mg/mL, about 110 mg/mL to about 380 mg/mL, about 110 mg/mL to about 360 mg/mL, about 110 mg/mL to about 340 mg/mL, about 110 mg/mL to about 320 mg/mL, about 110 mg/mL to about 300 mg/mL, about 110 mg/mL to about 280 mg/mL, about 110 mg/mL to about 260 mg/mL, about 110 mg/mL to about 240 mg/mL, about 110 mg/mL to about 220 mg/mL, about 110 mg/mL to about 200 mg/mL, about 110 mg/mL to about 190 mg/mL, about 110 mg/mL to about 180 mg/mL, about 110 mg/mL to about 170 mg/mL, about 110 mg/mL to about 160 mg/mL, about 110 mg/mL to about 140 mg/mL, about 110 mg/mL to about 130 mg/mL, about 110 mg/mL to about 120 mg/mL, about 120 mg/mL to about 500 mg/mL, about 120 mg/mL to about 480 mg/mL, about 120 mg/mL to about 460 mg/mL, about 120 mg/mL to about 440 mg/mL, about 120 mg/mL to about 420 mg/mL, about 120 mg/mL to about 400 mg/mL, about 120 mg/mL to about 380 mg/mL, about 120 mg/mL to about 360 mg/mL, about 120 mg/mL to about 340 mg/mL, about 120 mg/mL to about 320 mg/mL, about 120 mg/mL to about 300 mg/mL, about 120 mg/mL to about 280 mg/mL, about 120 mg/mL to about 260 mg/mL, about 120 mg/mL to about 240 mg/mL, about 120 mg/mL to about 220 mg/mL, about 120 mg/mL to about 200 mg/mL, about 120 mg/mL to about 190 mg/mL, about 120 mg/mL to about 180 mg/mL, about 120 mg/mL to about 170 mg/mL, about 120 mg/mL to about 160 mg/mL, about 120 mg/mL to about 140 mg/mL, about 120 mg/mL to about 130 mg/mL, about 130 mg/mL to about 500 mg/mL, about 130 mg/mL to about 480 mg/mL, about 130 mg/mL to about 460 mg/mL, about 130 mg/mL to about 440 mg/mL, about 130 mg/mL to about 420 mg/mL, about 130 mg/mL to about 400 mg/mL, about 130 mg/mL to about 380 mg/mL, about 130 mg/mL to about 360 mg/mL, about 130 mg/mL to about 340 mg/mL, about 130 mg/mL to about 320 mg/mL, about 130 mg/mL to about 300 mg/mL, about 130 mg/mL to about 280 mg/mL, about 130 mg/mL to about 260 mg/mL, about 130 mg/mL to about 240 mg/mL, about 130 mg/mL to about 220 mg/mL, about 130 mg/mL to about 200 mg/mL, about 130 mg/mL to about 190 mg/mL, about 130 mg/mL to about 180 mg/mL, about 130 mg/mL to about 170 mg/mL, about 130 mg/mL to about 160 mg/mL, about 130 mg/mL to about 140 mg/mL, about 140 mg/mL to about 500 mg/mL, about 140 mg/mL to about 480 mg/mL, about 140 mg/mL to about 460 mg/mL, about 140 mg/mL to about 440 mg/mL, about 140 mg/mL to about 420 mg/mL, about 140 mg/mL to about 400 mg/mL, about 140 mg/mL to about 380 mg/mL, about 140 mg/mL to about 360 mg/mL, about 140 mg/mL to about 340 mg/mL, about 140 mg/mL to about 320 mg/mL, about 140 mg/mL to about 300 mg/mL, about 140 mg/mL to about 280 mg/mL, about 140 mg/mL to about 260 mg/mL, about 140 mg/mL to about 240 mg/mL, about 140 mg/mL to about 220 mg/mL, about 140 mg/mL to about 200 mg/mL, about 140 mg/mL to about 190 mg/mL, about 140 mg/mL to about 180 mg/mL, about 140 mg/mL to about 170 mg/mL, about 140 mg/mL to about 160 mg/mL, about 160 mg/mL to about 500 mg/mL, about 160 mg/mL to about 480 mg/mL, about 160 mg/mL to about 460 mg/mL, about 160 mg/mL to about 440 mg/mL, about 160 mg/mL to about 420 mg/mL, about 160 mg/mL to about 400 mg/mL, about 160 mg/mL to about 380 mg/mL, about 160 mg/mL to about 360 mg/mL, about 160 mg/mL to about 340 mg/mL, about 160 mg/mL to about 320 mg/mL, about 160 mg/mL to about 300 mg/mL, about 160 mg/mL to about 280 mg/mL, about 160 mg/mL to about 260 mg/mL, about 160 mg/mL to about 240 mg/mL, about 160 mg/mL to about 220 mg/mL, about 160 mg/mL to about 200 mg/mL, about 160 mg/mL to about 190 mg/mL, about 160 mg/mL to about 180 mg/mL, about 160 mg/mL to about 170 mg/mL about 170 mg/mL to about 500 mg/mL, about 170 mg/mL to about 480 mg/mL, about 170 mg/mL to about 460 mg/mL, about 170 mg/mL to about 440 mg/mL, about 170 mg/mL to about 420 mg/mL, about 170 mg/mL to about 400 mg/mL, about 170 mg/mL to about 380 mg/mL, about 170 mg/mL to about 360 mg/mL, about 170 mg/mL to about 340 mg/mL, about 170 mg/mL to about 320 mg/mL, about 170 mg/mL to about 300 mg/mL, about 170 mg/mL to about 280 mg/mL, about 170 mg/mL to about 260 mg/mL, about 170 mg/mL to about 240 mg/mL, about 170 mg/mL to about 220 mg/mL, about 170 mg/mL to about 200 mg/mL, about 170 mg/mL to about 190 mg/mL, about 170 mg/mL to about 180 mg/mL, about 180 mg/mL to about 500 mg/mL, about 180 mg/mL to about 480 mg/mL, about 180 mg/mL to about 460 mg/mL, about 180 mg/mL to about 440 mg/mL, about 180 mg/mL to about 420 mg/mL, about 180 mg/mL to about 400 mg/mL, about 180 mg/mL to about 380 mg/mL, about 180 mg/mL to about 360 mg/mL, about 180 mg/mL to about 340 mg/mL, about 180 mg/mL to about 320 mg/mL, about 180 mg/mL to about 300 mg/mL, about 180 mg/mL to about 280 mg/mL, about 180 mg/mL to about 260 mg/mL, about 180 mg/mL to about 240 mg/mL, about 180 mg/mL to about 220 mg/mL, about 180 mg/mL to about 200 mg/mL, about 180 mg/mL to about 190 mg/mL, about 190 mg/mL to about 500 mg/mL, about 190 mg/mL to about 480 mg/mL, about 190 mg/mL to about 460 mg/mL, about 190 mg/mL to about 440 mg/mL, about 190 mg/mL to about 420 mg/mL, about 190 mg/mL to about 400 mg/mL, about 190 mg/mL to about 380 mg/mL, about 190 mg/mL to about 360 mg/mL, about 190 mg/mL to about 340 mg/mL, about 190 mg/mL to about 320 mg/mL, about 190 mg/mL to about 300 mg/mL, about 190 mg/mL to about 280 mg/mL, about 190 mg/mL to about 260 mg/mL, about 190 mg/mL to about 240 mg/mL, about 190 mg/mL to about 220 mg/mL, about 190 mg/mL to about 200 mg/mL, about 200 mg/mL to about 500 mg/mL, about 200 mg/mL to about 480 mg/mL, about 200 mg/mL to about 460 mg/mL, about 200 mg/mL to about 440 mg/mL, about 200 mg/mL to about 420 mg/mL, about 200 mg/mL to about 400 mg/mL, about 200 mg/mL to about 380 mg/mL, about 200 mg/mL to about 360 mg/mL, about 200 mg/mL to about 340 mg/mL, about 200 mg/mL to about 320 mg/mL, about 200 mg/mL to about 300 mg/mL, about 200 mg/mL to about 280 mg/mL, about 200 mg/mL to about 260 mg/mL, about 200 mg/mL to about 240 mg/mL, about 200 mg/mL to about 220 mg/mL, about 220 mg/mL to about 500 mg/mL, about 220 mg/mL to about 480 mg/mL, about 220 mg/mL to about 460 mg/mL, about 220 mg/mL to about 440 mg/mL, about 220 mg/mL to about 420 mg/mL, about 220 mg/mL to about 400 mg/mL, about 220 mg/mL to about 380 mg/mL, about 220 mg/mL to about 360 mg/mL, about 220 mg/mL to about 340 mg/mL, about 220 mg/mL to about 320 mg/mL, about 220 mg/mL to about 300 mg/mL, about 220 mg/mL to about 280 mg/mL, about 220 mg/mL to about 260 mg/mL, about 220 mg/mL to about 240 mg/mL, about 240 mg/mL to about 500 mg/mL, about 240 mg/mL to about 480 mg/mL, about 240 mg/mL to about 460 mg/mL, about 240 mg/mL to about 440 mg/mL, about 240 mg/mL to about 420 mg/mL, about 240 mg/mL to about 400 mg/mL, about 240 mg/mL to about 380 mg/mL, about 240 mg/mL to about 360 mg/mL, about 240 mg/mL to about 340 mg/mL, about 240 mg/mL to about 320 mg/mL, about 240 mg/mL to about 300 mg/mL, about 240 mg/mL to about 280 mg/mL, about 240 mg/mL to about 260 mg/mL, about 260 mg/mL to about 500 mg/mL, about 260 mg/mL to about 480 mg/mL, about 260 mg/mL to about 460 mg/mL, about 260 mg/mL to about 440 mg/mL, about 260 mg/mL to about 420 mg/mL, about 260 mg/mL to about 400 mg/mL, about 260 mg/mL to about 380 mg/mL, about 260 mg/mL to about 360 mg/mL, about 260 mg/mL to about 340 mg/mL, about 260 mg/mL to about 320 mg/mL, about 260 mg/mL to about 300 mg/mL, about 260 mg/mL to about 280 mg/mL, about 280 mg/mL to about 500 mg/mL, about 280 mg/mL to about 480 mg/mL, about 280 mg/mL to about 460 mg/mL, about 280 mg/mL to about 440 mg/mL, about 280 mg/mL to about 420 mg/mL, about 280 mg/mL to about 400 mg/mL, about 280 mg/mL to about 380 mg/mL, about 280 mg/mL to about 360 mg/mL, about 280 mg/mL to about 340 mg/mL, about 280 mg/mL to about 320 mg/mL, about 280 mg/mL to about 300 mg/mL, about 300 mg/mL to about 500 mg/mL, about 300 mg/mL to about 480 mg/mL, about 300 mg/mL to about 460 mg/mL, about 300 mg/mL to about 440 mg/mL, about 300 mg/mL to about 420 mg/mL, about 300 mg/mL to about 400 mg/mL, about 300 mg/mL to about 380 mg/mL, about 300 mg/mL to about 360 mg/mL, about 300 mg/mL to about 340 mg/mL, about 300 mg/mL to about 320 mg/mL, about 320 mg/mL to about 500 mg/mL, about 320 mg/mL to about 480 mg/mL, about 320 mg/mL to about 460 mg/mL, about 320 mg/mL to about 440 mg/mL, about 320 mg/mL to about 420 mg/mL, about 320 mg/mL to about 400 mg/mL, about 320 mg/mL to about 380 mg/mL, about 320 mg/mL to about 360 mg/mL, about 320 mg/mL to about 340 mg/mL, about 340 mg/mL to about 500 mg/mL, about 340 mg/mL to about 480 mg/mL, about 340 mg/mL to about 460 mg/mL, about 340 mg/mL to about 440 mg/mL, about 340 mg/mL to about 420 mg/mL, about 340 mg/mL to about 400 mg/mL, about 340 mg/mL to about 380 mg/mL, about 340 mg/mL to about 360 mg/mL, about 360 mg/mL to about 500 mg/mL, about 360 mg/mL to about 480 mg/mL, about 360 mg/mL to about 460 mg/mL, about 360 mg/mL to about 440 mg/mL, about 360 mg/mL to about 420 mg/mL, about 360 mg/mL to about 400 mg/mL, about 360 mg/mL to about 380 mg/mL, about 380 mg/mL to about 500 mg/mL, about 380 mg/mL to about 480 mg/mL, about 380 mg/mL to about 460 mg/mL, about 380 mg/mL to about 440 mg/mL, about 380 mg/mL to about 420 mg/mL, about 380 mg/mL to about 400 mg/mL, about 400 mg/mL to about 500 mg/mL, about 400 mg/mL to about 480 mg/mL, about 400 mg/mL to about 460 mg/mL, about 400 mg/mL to about 440 mg/mL, about 400 mg/mL to about 420 mg/mL, about 420 mg/mL to about 500 mg/mL, about 420 mg/mL to about 480 mg/mL, about 420 mg/mL to about 460 mg/mL, about 420 mg/mL to about 440 mg/mL, about 440 mg/mL to about 500 mg/mL, about 440 mg/mL to about 480 mg/mL, about 440 mg/mL to about 460 mg/mL, about 460 mg/mL to about 500 mg/mL, about 460 mg/mL to about 480 mg/mL, or about 480 mg/mL to about 500 mg/mL, of an antibody or an antigen-binding antibody fragment (e.g., any of the exemplary antibodies or antigen-binding antibody fragments described herein).

The term “antibody” as used herein is used broadly to mean any polypeptide that includes an antigen-binding domain. Non-limiting examples of types of antibodies are described herein. Additional examples of antibodies are known in the art.

The term “antigen-binding antibody fragment” refers to a fragment of a mammalian (e.g., human) IgG1, IgG2, IgG3, IgG4, IgM, IgE, or IgA that retains its ability to bind specifically to an antigen. Non-limiting examples of antigen-binding antibody fragments are described herein. Additional examples of antigen-binding antibody fragments are known in the art.

In some embodiments, an antibody can be a VHH domain, a VNAR domain, a scFv, a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv.

A V_{H}H domain is a single monomeric variable antibody domain that can be found in camelids. A V_{NAR} domain is a single monomeric variable antibody domain that can be found in cartilaginous fish. Non-limiting aspects of VIM domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments, an antibody can be a VHH-scAb, a VHH-Fab, a Dual scFab, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, and a scFv1-PEG-scFv2.

Non-limiting examples of an antigen-binding antibody fragments include an Fv fragment, a Fab fragment, a F(ab′)2 fragment, and a Fab′ fragment. Additional examples of antigen-binding antibody fragments include any antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment includes a non-covalently-linked dimer of one heavy chain variable domain and one light chain variable domain.

A “Fab” fragment includes, the constant domain of the light chain and the first constant domain (C_{H1}) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

An antibody or an antigen-binding antibody fragment can bind to its epitope or antigen with a dissociation equilibrium constant (K_{D}) of less than 1×10⁻⁷ M, less than 1×10⁻⁸M, less than 1×10⁻⁹M, less than 1×10⁻¹° M, less than 1×10⁻¹¹M, less than 1×10⁻¹² M, or less than 1×10⁻¹³ M. In some embodiments, the antibody or the antigen-binding antibody fragment can bind to its antigen or epitope with a K_{D} of about 1×10⁻³ M to about 1×10⁻⁵M, about 1×10⁻⁴M to about 1×10⁻⁶M, about 1×10⁻⁵M to about 1×10⁻⁷ M, about 1×10⁻⁶M to about 1×10⁻⁸M, about 1×10⁻⁷M to about 1×10⁻⁹M, about 1×10⁻⁸M to about 1×10⁻¹⁰ M, or about 1×10⁻⁹M to about 1×10⁻¹¹M (inclusive).

In some examples, the antibody can be a mAb (e.g., a monoclonal human or humanized antibody).

In some embodiments, the mAb can have an Fc region comprising one or more amino acid substitutions that result in low CH2 domain unfolding temperature compared to an antibody having a wildtype Fc region. In some embodiments, the mAb can have an Fc region comprising one or more amino acid substitutions that decrease the stability of the antibody, e.g., as compared to the stability of a similar antibody lacking the one or more amino acid substitutions.

In some embodiments, the mAb can be an IgG1, IgG2, IgG3, or IgG4 antibody (e.g., a human or humanized antibody). In some preferred embodiments, the mAb is an IgG1 or IgG4 antibody.

In some embodiments, the mAb is an anti-C—X—C motif chemokine receptor 3 (CXCR3) mAb (e.g., a human or humanized antibody). In some embodiments, the anti-CXCR3 mAb comprises a heavy chain comprising or consisting of a sequence that is at least 80%, at least 85%, at least 90%, at least 95%, at least 99% identical, or 100% identical to SEQ ID NO: 1 and a light chain comprising or consisting of a sequence that is at least 80% identical, at least 85% identical, at least 90% identical, at least 95% identical, at least 99% identical, or 100% identical SEQ ID NO: 2. In some embodiments, the anti-CXCR3 antibody includes the three CDRs present in SEQ ID NO: 1 and the three CDRs present in SEQ ID NO: 2.

In some embodiments, the mAb is an anti-cluster of differentiation 38 (CD38) mAb (e.g., a human or humanized anti-CD38 antibody). In some embodiments, the anti-CD38 mAb comprises a heavy chain comprising or consisting of a sequence that is at least 80%, at least 85%, at least 90%, at least 95%, at least 99% identical, or 100% identical to SEQ ID NO: 3 and a light chain comprising or consisting of a sequence that is at least 80% identical, at least 85% identical, at least 90% identical, at least 95% identical, at least 99% identical, or 100% identical SEQ ID NO: 4. In some embodiments, the anti-CD38 antibody includes the three CDRs present in SEQ ID NO: 3 and the three CDRs present in SEQ ID NO: 4.

In some embodiments, the mAb is an anti-cluster of differentiation 38 (CD38)-Fc engineered mAb (e.g., a human or humanized antibody). In some embodiments, the anti-CD38-Fc engineered mAb comprises a heavy chain comprising or consisting of a sequence that is at least 80%, at least 85%, at least 90%, at least 95%, at least 99% identical, or 100% identical to SEQ ID NO: 5 and a light chain comprising or consisting of a sequence that is at least 80% identical, at least 85% identical, at least 90% identical, at least 95% identical, at least 99% identical, or 100% identical SEQ ID NO: 6. In some embodiments, the anti-CD38-Fc engineered mAb includes the three CDRs present in SEQ ID NO: 5 and the three CDRs present in SEQ ID NO: 6.

In some embodiments, the mAb is an anti-carcinoembryonic antigen-related cell adhesion molecule 5 (CEACAM5) mAb (e.g., a human or humanized antibody). In some embodiments, the anti-CEACAM5 mAb comprises a heavy chain comprising or consisting of a sequence that is at least 80%, at least 85%, at least 90%, at least 95%, at least 99% identical, or 100% identical to SEQ ID NO: 9 and a light chain comprising or consisting of a sequence that is at least 80% identical, at least 85% identical, at least 90% identical, at least 95% identical, at least 99% identical, or 100% identical SEQ ID NO: 10. In some embodiments, the anti-CEACAM5 antibody includes the three CDRs present in SEQ ID NO: 9 and the three CDRs present in SEQ ID NO: 10.

In some embodiments, the mAb is an anti-carcinoembryonic antigen-related cell adhesion molecule 5 (CEACAM5)-Fc engineered mAb (e.g., a human or humanized antibody). In some embodiments, the anti-CEACAM5-Fc engineered mAb comprises a heavy chain comprising or consisting of a sequence that is at least 80%, at least 85%, at least 90%, at least 95%, at least 99% identical, or 100% identical to SEQ ID NO: 9 and a light chain comprising or consisting of a sequence that is at least 80% identical, at least 85% identical, at least 90% identical, at least 95% identical, at least 99% identical, or 100% identical SEQ ID NO: 10. In some embodiments, the anti-CEACAM5-Fc engineered mAb includes the three CDRs present in SEQ ID NO: 9 and the three CDRs present in SEQ ID NO: 10.

In some embodiments, the mAb is an anti-carcinoembryonic antigen-related cell adhesion molecule 5 (CEACAM5)-Fc engineered mAb (e.g., a human or humanized antibody). In some embodiments, the anti-CEACAM5-Fc engineered mAb comprises a heavy chain comprising or consisting of a sequence that is at least 80%, at least 85%, at least 90%, at least 95%, at least 99% identical, or 100% identical to SEQ ID NO: 11 and a light chain comprising or consisting of a sequence that is at least 80% identical, at least 85% identical, at least 90% identical, at least 95% identical, at least 99% identical, or 100% identical SEQ ID NO: 12. In some embodiments, the anti-CEACAM5-Fc engineered mAb includes the three CDRs present in SEQ ID NO: 11 and the three CDRs present in SEQ ID NO: 12.

In some embodiments, the mAb is an anti-carcinoembryonic antigen-related cell adhesion molecule 5 (CEACAM5)-Fc engineered mAb (e.g., a human or humanized antibody). In some embodiments, the anti-CEACAM5-Fc engineered mAb comprises a heavy chain comprising or consisting of a sequence that is at least 80%, at least 85%, at least 90%, at least 95%, at least 99% identical, or 100% identical to SEQ ID NO: 13 and a light chain comprising or consisting of a sequence that is at least 80% identical, at least 85% identical, at least 90% identical, at least 95% identical, at least 99% identical, or 100% identical SEQ ID NO: 14. In some embodiments, the anti-CEACAM5-Fc engineered mAb includes the three CDRs present in SEQ ID NO: 13 and the three CDRs present in SEQ ID NO: 14.

In some embodiments, an antibody can be conjugated to a drug (e.g., a chemotherapeutic drug, a small molecule), a toxin, or a radioisotope. In some embodiments, an antibody can be conjugated to a drug through a linker. Non-limiting examples of linkers include: hydrazone linkers, peptide linkers, disulfide linkers, thioether linker. See, e.g., Carter et al. (2008) Cancer J. 14(3): 154-69; Sanderson et al. (2005) Clin. Cancer Res. 11(2 Pt1): 843-852; Chari et al (2008) Acc Chem Res. 41(1): 98-107; Oflazoglu et al. (2008) Clin. Cancer Res. 14(19): 6171-6180; and Lu et al. (2016) Int. J. Mol. Sci. 17(4): 561.

An antibody can be produced by introducing into a cell a nucleic acid sequence encoding the antibody to produce a recombinant cell; and culturing the recombinant cell under conditions sufficient for the expression of the antibody. In some embodiments, the introducing step includes introducing into a cell an expression vector including a sequence encoding the antibody to produce a recombinant cell.

An antigen described herein can be produced by any cell, e.g., a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells.

Methods of culturing cells are well known in the art. Cells can be maintained in vitro under conditions that favor proliferation, differentiation and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

Provided herein are methods that further include isolation of the antibody from a cell (e.g., a eukaryotic cell) using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, size exclusion chromatography).

### Buffers

The formulations described herein can include a buffer (e.g., one or more buffers) (e.g., any of the non-limiting buffers described herein or known in the art). In some embodiments, the antibody or antigen-binding antibody fragment present in the formulation does not significantly buffer the pH of the formulation.

Non-limiting examples of a buffer (e.g., one or more buffers) that can be present in any of the formulations described herein include: acetate, succinate, gluconate, histidine, citrate, phosphate, and Tris. In some embodiments of any of the formulations described herein, the formulation can include acetate, histidine, or phosphate. Additional examples of buffers that can be present in any of the formulations described herein are known in the art.

The final concentration of a buffer (or a final total concentration of one or more buffers) in any of the formulations described herein can be about 0.01 mM to about 100 mM, about 0.01 mM to about 95 mM, about 0.01 mM to about 90 mM, about 0.01 mM to about 85 mM, about 0.01 mM to about 80 mM, about 0.01 mM to about 75 mM, about 0.01 mM to about 70 mM, about 0.01 mM to about 65 mM, about 0.01 mM to about 60 mM, about 0.01 mM to about 55 mM, about 0.01 mM to about 50 mM, about 0.01 mM to about 45 mM, about 0.01 mM to about 40 mM, about 0.01 mM to about 35 mM, about 0.01 mM to about 30 mM, about 0.01 mM to about 25 mM, about 0.01 mM to about 20 mM, about 0.01 mM to about 15 mM, about 0.01 mM to about 10 mM, about 0.01 mM to about 9 mM, about 0.01 mM to about 8.5 mM, about 0.01 mM to about 8 mM, about 0.01 mM to about 7.5 mM, about 0.01 mM to about 7 mM, about 0.01 mM to about 6.5 mM, about 0.01 mM to about 6 mM, about 0.01 mM to about 5 mM, about 0.01 mM to about 4.5 mM, about 0.01 mM to about 4 mM, about 0.01 mM to about 3.5 mM, about 0.01 mM to about 3 mM, about 0.01 mM to about 2.5 mM, about 0.01 mM to about 2 mM, about 0.01 mM to about 1.5 mM, about 0.01 mM to about 1 mM, about 0.01 mM to about 0.9 mM, about 0.01 mM to about 0.8 mM, about 0.01 mM to about 0.7 mM, about 0.01 mM to about 0.6 mM, about 0.01 mM to about 0.5 mM, about 0.01 mM to about 0.4 mM, about 0.01 mM to about 0.3 mM, about 0.01 mM to about 0.2 mM, about 0.01 mM to about 0.1 mM, about 0.1 mM to about 100 mM, about 0.1 mM to about 95 mM, about 0.1 mM to about 90 mM, about 0.1 mM to about 85 mM, about 0.1 mM to about 80 mM, about 0.1 mM to about 75 mM, about 0.1 mM to about 70 mM, about 0.1 mM to about 65 mM, about 0.1 mM to about 60 mM, about 0.1 mM to about 55 mM, about 0.1 mM to about 50 mM, about 0.1 mM to about 45 mM, about 0.1 mM to about 40 mM, about 0.1 mM to about 35 mM, about 0.1 mM to about 30 mM, about 0.1 mM to about 25 mM, about 0.1 mM to about 20 mM, about 0.1 mM to about 15 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 0.1 mM to about 0.9 mM, about 0.1 mM to about 0.8 mM, about 0.1 mM to about 0.7 mM, about 0.1 mM to about 0.6 mM, about 0.1 mM to about 0.5 mM, about 0.1 mM to about 0.4 mM, about 0.1 mM to about 0.3 mM, about 0.1 mM to about 0.2 mM, about 0.2 mM to about 100 mM, about 0.2 mM to about 95 mM, about 0.2 mM to about 90 mM, about 0.2 mM to about 85 mM, about 0.2 mM to about 80 mM, about 0.2 mM to about 75 mM, about 0.2 mM to about 70 mM, about 0.2 mM to about 65 mM, about 0.2 mM to about 60 mM, about 0.2 mM to about 55 mM, about 0.2 mM to about 50 mM, about 0.2 mM to about 45 mM, about 0.2 mM to about 40 mM, about 0.2 mM to about 35 mM, about 0.2 mM to about 30 mM, about 0.2 mM to about 25 mM, about 0.2 mM to about 20 mM, about 0.2 mM to about 15 mM, about 0.2 mM to about 10 mM, about 0.2 mM to about 9 mM, about 0.2 mM to about 8 mM, about 0.2 mM to about 7 mM, about 0.2 mM to about 6 mM, about 0.2 mM to about 5 mM, about 0.2 mM to about 4 mM, about 0.2 mM to about 3 mM, about 0.2 mM to about 2 mM, about 0.2 mM to about 1 mM, about 0.2 mM to about 0.9 mM, about 0.2 mM to about 0.8 mM, about 0.2 mM to about 0.7 mM, about 0.2 mM to about 0.6 mM, about 0.2 mM to about 0.5 mM, about 0.2 mM to about 0.4 mM, about 0.2 mM to about 0.3 mM, about 0.5 mM to about 100 mM, about 0.5 mM to about 95 mM, about 0.5 mM to about 90 mM, about 0.5 mM to about 85 mM, about 0.5 mM to about 80 mM, about 0.5 mM to about 75 mM, about 0.5 mM to about 70 mM, about 0.5 mM to about 65 mM, about 0.5 mM to about 60 mM, about 0.5 mM to about 55 mM, about 0.5 mM to about 50 mM, about 0.5 mM to about 45 mM, about 0.5 mM to about 40 mM, about 0.5 mM to about 35 mM, about 0.5 mM to about 30 mM, about 0.5 mM to about 25 mM, about 0.5 mM to about 20 mM, about 0.5 mM to about 15 mM, about 0.5 mM to about 10 mM, about 0.5 mM to about 0.9 mM, about 0.5 mM to about 0.8 mM, about 0.5 mM to about 0.7 mM, about 0.5 mM to about 0.5 mM to about 0.6 mM, about 0.5 mM to about 5 mM, about 0.5 mM to about 4 mM, about 0.5 mM to about 3 mM, about 0.5 mM to about 2 mM, about 0.5 mM to about 1 mM, about 0.5 mM to about 0.9 mM, about 0.5 mM to about 0.8 mM, about 0.5 mM to about 0.7 mM, about 0.5 mM to about 0.6 mM, about 1 mM to about 100 mM, about 1 mM to about 95 mM, about 1 mM to about 90 mM, about 1 mM to about 85 mM, about 1 mM to about 80 mM, about 1 mM to about 75 mM, about 1 mM to about 70 mM, about 1 mM to about 65 mm, about 1 mM to about 60 mM, about 1 mM to about 55 mM, about 1 mM to about 50 mM, about 1 mM to about 45 mM, about 1 mM to about 40 mM, about 1 mM to about 35 mM, about 1 mM to about 30 mM, about 1 mM to about 25 mM, about 1 mM to about 20 mM, about 1 mM to about 15 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, about 1 mM to about 1.8 mM, about 1 mM to about 1.6 mM, about 1 mM to about 1.4 mM, about 1 mM to about 1.2 mM, about 2 mM to about 100 mM, about 2 mM to about 95 mM, about 2 mM to about 90 mM, about 2 mM to about 85 mM, about 2 mM to about 80 mM, about 2 mM to about 75 mM, about 2 mM to about 70 mM, about 2 mM to about 65 mM, about 2 mM to about 60 mM, about 2 mM to about 55 mM, about 2 mM to about 50 mM, about 2 mM to about 45 mM, about 2 mM to about 40 mM, about 2 mM to about 35 mM, about 2 mM to about 30 mM, about 2 mM to about 25 mM, about 2 mM to about 20 mM, about 2 mM to about 15 mM, about 2 mM to about 10 mM, about 2 mM to about 9 mM, about 2 mM to about 8 mM, about 2 mM to about 7 mM, about 2 mM to about 6 mM, about 2 mM to about 5 mM, about 2 mM to about 4 mM, about 2 mM to about 3 mM, about 5 mM to about 100 mM, about 5 mM to about 95 mM, about 5 mM to about 90 mM, about 5 mM to about 85 mM, about 5 mM to about 80 mM, about 5 mM to about 75 mM, about 5 mM to about 70 mM, about 5 mM to about 65 mM, about 5 mM to about 60 mM, about 5 mM to about 55 mM, about 5 mM to about 50 mM, about 5 mM to about 45 mM, about 5 mM to about 40 mM, about 5 mM to about 35 mM, about 5 mM to about 30 mM, about 5 mM to about 25 mM, about 5 mM to about 20 mM, about 5 mM to about 15 mM, about 5 mM to about 10 mM, about 5 mM to about 9 mM, about 5 mM to about 8 mM, about 5 mM to about 7 mM, about 5 mM to about 6 mM, about 10 mM to about 100 mM, about 10 mM to about 95 mM, about 10 mM to about 90 mM, about 10 mM to about 85 mM, about 10 mM to about 80 mM, about 10 mM to about 75 mM, about 10 mM to about 70 mM, about 10 mM to about 65 mM, about 10 mM to about 60 mM, about 10 mM to about 55 mM, about 10 mM to about 50 mM, about 10 mM to about 45 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 10 mM to about 14 mM, about 10 mM to about 13 mM, about 10 mM to about 12 mM, about 10 mM to about 11 mM, about 15 mM to about 100 mM, about 15 mM to about 95 mM, about 15 mM to about 90 mM, about 15 mM to about 85 mM, about 15 mM to about 80 mM, about 15 mM to about 75 mM, about 15 mM to about 70 mM, about 15 mM to about 65 mM, about 15 mM to about 60 mM, about 15 mM to about 55 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 20 mM to about 100 mM, about 20 mM to about 95 mM, about 20 mM to about 90 mM, about 20 mM to about 85 mM, about 20 mM to about 80 mM, about 20 mM to about 75 mM, about 20 mM to about 70 mM, about 20 mM to about 65 mM, about 20 mM to about 60 mM, about 20 mM to about 55 mM, about 20 mM to about 50 mM, about 20 mM to about 45 mM, about 20 mM to about 40 mM, about 20 mM to about 35 mM, about 20 mM to about 30 mM, about 20 mM to about 25 mM, about 20 mM to about 24 mM, about 20 mM to about 23 mM, about 20 mM to about 22 mM, about 20 mM to about 21 mM, about 25 mM to about 100 mM, about 25 mM to about 95 mM, about 25 mM to about 90 mM, about 25 mM to about 85 mM, about 25 mM to about 80 mM, about 25 mM to about 75 mM, about 25 mM to about 70 mM, about 25 mM to about 65 mM, about 25 mM to about 60 mM, about 25 mM to about 55 mM, about 25 mM to about 50 mM, about 25 mM to about 45 mM, about 25 mM to about 40 mM, about 25 mM to about 35 mM, about 25 mM to about 30 mM, about 25 mM to about 29 mM, about 25 mM to about 28 mM, about 25 mM to about 27 mM, about 25 mM to about 26 mM, about 30 mM to about 100 mM, about 30 mM to about 95 mM, about 30 mM to about 90 mM, about 30 mM to about 85 mM, about 30 mM to about 80 mM, about 30 mM to about 75 mM, about 30 mM to about 70 mM, about 30 mM to about 65 mM, about 30 mM to about 60 mM, about 30 mM to about 55 mM, about 30 mM to about 50 mM, about 30 mM to about 45 mM, about 30 mM to about 40 mM, about 30 mM to about 35 mM, about 30 mM to about 34 mM, about 30 mM to about 33 mM, about 30 mM to about 32 mM, about 30 mM to about 31 mM, about 35 mM to about 100 mM, about 35 mM to about 95 mM, about 35 mM to about 90 mM, about 35 mM to about 85 mM, about 35 mM to about 80 mM, about 35 mM to about 75 mM, about 35 mM to about 70 mM, about 35 mM to about 65 mM, about 35 mM to about 60 mM, about 35 mM to about 55 mM, about 35 mM to about 50 mM, about 35 mM to about 45 mM, about 35 mM to about 40 mM, about 35 mM to about 39 mM, about 35 mM to about 38 mM, about 35 mM to about 37 mM, about 35 mM to 36 mM, about 40 mM to about 100 mM, about 40 mM to about 95 mM, about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, about 40 mM to about 50 mM, about 40 mM to about 45 mM, about 40 mM to about 44 mM, about 40 mM to about 43 mM, about 40 mM to about 42 mM, about 40 mM to about 41 mM, about 45 mM to about 100 mM, about 45 mM to about 95 mM, about 45 mM to about 90 mM, about 45 mM to about 85 mM, about 45 mM to about 80 mM, about 45 mM to about 75 mM, about 45 mM to about 70 mM, about 45 mM to about 65 mM, about 45 mM to about 60 mM, about 45 mM to about 55 mM, about 45 mM to about 50 mM, about 45 mM to about 49 mM, about 45 mM to about 48 mM, about 45 mM to about 47 mM, about 45 mM to about 46 mM, about 50 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, about 50 mM to about 55 mM, about 50 mM to about 54 mM, about 50 mM to about 53 mM, about 50 mM to about 52 mM, about 50 mM to about 51 mM, about 60 mM to about 100 mM, about 60 mM to about 95 mM, about 60 mM to about 90 mM, about 60 mM to about 85 mM, about 60 mM to about 80 mM, about 60 mM to about 75 mM, about 60 mM to about 70 mM, about 60 mM to about 65 mM, about 60 mM to about 64 mM, about 60 mM to about 63 mM, about 60 mM to about 62 mM, about 60 mM to about 61 mM, about 70 mM to about 100 mM, about 70 mM to about 95 mM, about 70 mM to about 90 mM, about 70 mM to about 85 mM, about 70 mM to about 80 mM, about 70 mM to about 75 mM, about 70 mM to about 74 mM, about 70 mM to about 73 mM, about 70 mM to about 72 mM, about 70 mM to about 71 mM, about 90 mM to about 100 mM, about 90 mM to about 95 mM, about 90 mM to about 94 mM, about 90 mM to about 93 mM, about 90 mM to about 92 mM, or about 90 mM to about 91 mM.

### Salts

The aqueous antibody formulations described herein include a salt (e.g., one or more salts) selected from the group of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate. In some examples, the aqueous antibody formulations described herein include a salt (e.g., one or more salts) selected from the group of: magnesium glutamate, magnesium acetate, magnesium aspartate, and magnesium sulfate.

The final concentration of a salt (or the final total concentration of one or more salts) selected from the group of magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate in any of the formulations described herein can be about 0.01 mM to about 750 mM (or any of the subranges of this range described herein). In some embodiments, the final concentration of a salt in any of the formulations described herein can be about 0.01 mM to about 750 mM, about 0.01 mM to about 700 mM, about 0.01 mM to about 650 mM, about 0.01 mM to about 600 mM, about 0.01 mM to about 550 mM, about 0.01 mM to about 500 mM, about 0.01 mM to about 450 mM, about 0.01 mM to about 400 mM, about 0.01 mM to about 350 mM, about 0.01 mM to about 300 mM, about 0.01 mM to about 290 mM, about 0.01 mM to about 280 mM, about 0.01 mM to about 270 mM, about 0.01 mM to about 260 mM, about 0.01 mM to about 250 mM, about 0.01 mM to about 240 mM, about 0.01 mM to about 230 mM, about 0.01 mM to about 220 mM, about 0.01 mM to about 210 mM, about 0.01 mM to about 200 mM, about 0.01 mM to about 190 mM, about 0.01 mM to about 180 mM, about 0.01 mM to about 170 mM, about 0.01 mM to about 160 mM, about 0.01 mM to about 150 mM, about 0.01 mM to about 140 mM, about 0.01 mM to about 130 mM, about 0.01 mM to about 120 mM, about 0.01 mM to about 110 mM, about 0.01 mM to about 100 mM, about 0.01 mM to about 95 mM, about 0.01 mM to about 90 mM, about 0.01 mM to about 85 mM, about 0.01 mM to about 80 mM, about 0.01 mM to about 75 mM, about 0.01 mM to about 70 mM, about 0.01 mM to about 65 mM, about 0.01 mM to about 60 mM, about 0.01 mM to about 55 mM, about 0.01 mM to about 50 mM, about 0.01 mM to about 45 mM, about 0.01 mM to about 40 mM, about 0.01 mM to about 35 mM, about 0.01 mM to about 30 mM, about 0.01 mM to about 25 mM, about 0.01 mM to about 20 mM, about 0.01 mM to about 15 mM, about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.01 mM to about 0.5 mM, about 0.01 mM to about 0.2 mM, about 0.01 mM to about 0.1 mM, about 0.1 mM to about 500 mM, about 0.1 mM to about 450 mM, about 0.1 mM to about 400 mM, about 0.1 mM to about 350 mM, about 0.1 mM to about 300 mM, about 0.1 mM to about 290 mM, about 0.1 mM to about 280 mM, about 0.1 mM to about 270 mM, about 0.1 mM to about 260 mM, about 0.1 mM to about 250 mM, about 0.1 mM to about 240 mM, about 0.1 mM to about 230 mM, about 0.1 mM to about 220 mM, about 0.1 mM to about 210 mM, about 0.1 mM to about 200 mM, about 0.1 mM to about 190 mM, about 0.1 mM to about 180 mM, about 0.1 mM to about 170 mM, about 0.1 mM to about 160 mM, about 0.1 mM to about 150 mM, about 0.1 mM to about 140 mM, about 0.1 mM to about 130 mM, about 0.1 mM to about 120 mM, about 0.1 mM to about 110 mM, about 0.1 mM to about 100 mM, about 0.1 mM to about 95 mM, about 0.1 mM to about 90 mM, about 0.1 mM to about 85 mM, about 0.1 mM to about 80 mM, about 0.1 mM to about 75 mM, about 0.1 mM to about 70 mM, about 0.1 mM to about 65 mM, about 0.1 mM to about 60 mM, about 0.1 mM to about 55 mM, about 0.1 mM to about 50 mM, about 0.1 mM to about 45 mM, about 0.1 mM to about 40 mM, about 0.1 mM to about 35 mM, about 0.1 mM to about 30 mM, about 0.1 mM to about 25 mM, about 0.1 mM to about 20 mM, about 0.1 mM to about 15 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 0.1 mM to about 0.5 mM, about 0.1 mM to about 0.2 mM, 0.2 mM to about 750 mM, about 0.2 mM to about 700 mM, about 0.2 mM to about 650 mM, about 0.2 mM to about 600 mM, about 0.2 mM to about 550 mM, about 0.2 mM to about 500 mM, about 0.2 mM to about 450 mM, about 0.2 mM to about 400 mM, about 0.2 mM to about 350 mM, about 0.2 mM to about 300 mM, about 0.2 mM to about 290 mM, about 0.2 mM to about 280 mM, about 0.2 mM to about 270 mM, about 0.2 mM to about 260 mM, about 0.2 mM to about 250 mM, about 0.2 mM to about 240 mM, about 0.2 mM to about 230 mM, about 0.2 mM to about 220 mM, about 0.2 mM to about 210 mM, about 0.2 mM to about 200 mM, about 0.2 mM to about 190 mM, about 0.2 mM to about 180 mM, about 0.2 mM to about 170 mM, about 0.2 mM to about 160 mM, about 0.2 mM to about 150 mM, about 0.2 mM to about 140 mM, about 0.2 mM to about 130 mM, about 0.2 mM to about 120 mM, about 0.2 mM to about 110 mM, about 0.2 mM to about 100 mM, about 0.2 mM to about 95 mM, about 0.2 mM to about 90 mM, about 0.2 mM to about 85 mM, about 0.2 mM to about 80 mM, about 0.2 mM to about 75 mM, about 0.2 mM to about 70 mM, about 0.2 mM to about 65 mM, about 0.2 mM to about 60 mM, about 0.2 mM to about 55 mM, about 0.2 mM to about 50 mM, about 0.2 mM to about 45 mM, about 0.2 mM to about 40 mM, about 0.2 mM to about 35 mM, about 0.2 mM to about 30 mM, about 0.2 mM to about 25 mM, about 0.2 mM to about 20 mM, about 0.2 mM to about 15 mM, about 0.2 mM to about 10 mM, about 0.2 mM to about 9 mM, about 0.2 mM to about 8 mM, about 0.2 mM to about 7 mM, about 0.2 mM to about 6 mM, about 0.2 mM to about 5 mM, about 0.2 mM to about 4 mM, about 0.2 mM to about 3 mM, about 0.2 mM to about 2 mM, about 0.2 mM to about 1 mM, about 0.2 mM to about 0.5 mM, about 0.5 mM to about 750 mM, about 0.5 mM to about 700 mM, about 0.5 mM to about 650 mM, about 0.5 mM to about 600 mM, about 0.5 mM to about 550 mM, about 0.5 mM to about 500 mM, about 0.5 mM to about 450 mM, about 0.5 mM to about 400 mM, about 0.5 mM to about 350 mM, about 0.5 mM to about 300 mM, about 0.5 mM to about 290 mM, about 0.5 mM to about 280 mM, about 0.5 mM to about 270 mM, about 0.5 mM to about 260 mM, about 0.5 mM to about 250 mM, about 0.5 mM to about 240 mM, about 0.5 mM to about 230 mM, about 0.5 mM to about 220 mM, about 0.5 mM to about 210 mM, about 0.5 mM to about 200 mM, about 0.5 mM to about 190 mM, about 0.5 mM to about 180 mM, about 0.5 mM to about 170 mM, about 0.5 mM to about 160 mM, about 0.5 mM to about 150 mM, about 0.5 mM to about 140 mM, about 0.5 mM to about 130 mM, about 0.5 mM to about 120 mM, about 0.5 mM to about 110 mM, about 0.5 mM to about 100 mM, about 0.5 mM to about 95 mM, about 0.5 mM to about 90 mM, about 0.5 mM to about 85 mM, about 0.5 mM to about 80 mM, about 0.5 mM to about 75 mM, about 0.5 mM to about 70 mM, about 0.5 mM to about 65 mM, about 0.5 mM to about 60 mM, about 0.5 mM to about 55 mM, about 0.5 mM to about 50 mM, about 0.5 mM to about 45 mM, about 0.5 mM to about 40 mM, about 0.5 mM to about 35 mM, about 0.5 mM to about 30 mM, about 0.5 mM to about 25 mM, about 0.5 mM to about 20 mM, about 0.5 mM to about 15 mM, about 0.5 mM to about 10 mM, about 0.5 mM to about 9 mM, about 0.5 mM to about 8 mM, about 0.5 mM to about 7 mM, about 0.5 mM to about 6 mM, about 0.5 mM to about 5 mM, about 0.5 mM to about 4 mM, about 0.5 mM to about 3 mM, about 0.5 mM to about 2 mM, about 0.5 mM to about 1 mM, about 1 mM to about 750 mM, about 1 mM to about 700 mM, about 1 mM to about 650 mM, about 1 mM to about 600 mM, about 1 mM to about 550 mM, about 1 mM to about 500 mM, about 1 mM to about 450 mM, about 1 mM to about 400 mM, about 1 mM to about 350 mM, about 1 mM to about 300 mM, about 1 mM to about 290 mM, about 1 mM to about 280 mM, about 1 mM to about 270 mM, about 1 mM to about 260 mM, about 1 mM to about 250 mM, about 1 mM to about 240 mM, about 1 mM to about 230 mM, about 1 mM to about 220 mM, about 1 mM to about 210 mM, about 1 mM to about 200 mM, about 1 mM to about 190 mM, about 1 mM to about 180 mM, about 1 mM to about 170 mM, about 1 mM to about 160 mM, about 1 mM to about 150 mM, about 1 mM to about 140 mM, about 1 mM to about 130 mM, about 1 mM to about 120 mM, about 1 mM to about 110 mM, about 1 mM to about 100 mM, about 1 mM to about 95 mM, about 1 mM to about 90 mM, about 1 mM to about 85 mM, about 1 mM to about 80 mM, about 1 mM to about 75 mM, about 1 mM to about 70 mM, about 1 mM to about 65 mM, about 1 mM to about 60 mM, about 1 mM to about 55 mM, about 1 mM to about 50 mM, about 1 mM to about 45 mM, about 1 mM to about 40 mM, about 1 mM to about 35 mM, about 1 mM to about 30 mM, about 1 mM to about 25 mM, about 1 mM to about 20 mM, about 1 mM to about 15 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, about 2 mM to about 750 mM, about 2 mM to about 700 mM, about 2 mM to about 650 mM, about 2 mM to about 600 mM, about 2 mM to about 550 mM, about 2 mM to about 500 mM, about 2 mM to about 450 mM, about 2 mM to about 400 mM, about 2 mM to about 350 mM, about 2 mM to about 300 mM, about 2 mM to about 290 mM, about 2 mM to about 280 mM, about 2 mM to about 270 mM, about 2 mM to about 260 mM, about 2 mM to about 250 mM, about 2 mM to about 240 mM, about 2 mM to about 230 mM, about 2 mM to about 220 mM, about 2 mM to about 210 mM, about 2 mM to about 200 mM, about 2 mM to about 190 mM, about 2 mM to about 180 mM, about 2 mM to about 170 mM, about 2 mM to about 160 mM, about 2 mM to about 150 mM, about 2 mM to about 140 mM, about 2 mM to about 130 mM, about 2 mM to about 120 mM, about 2 mM to about 110 mM, about 2 mM to about 100 mM, about 2 mM to about 95 mM, about 2 mM to about 90 mM, about 2 mM to about 85 mM, about 2 mM to about 80 mM, about 2 mM to about 75 mM, about 2 mM to about 70 mM, about 2 mM to about 65 mM, about 2 mM to about 60 mM, about 2 mM to about 55 mM, about 2 mM to about 50 mM, about 2 mM to about 45 mM, about 2 mM to about 40 mM, about 2 mM to about 35 mM, about 2 mM to about 30 mM, about 2 mM to about 25 mM, about 2 mM to about 20 mM, about 2 mM to about 15 mM, about 2 mM to about 10 mM, about 2 mM to about 9 mM, about 2 mM to about 8 mM, about 2 mM to about 7 mM, about 2 mM to about 6 mM, about 2 mM to about 5 mM, about 5 mM to about 750 mM, about 5 mM to about 700 mM, about 5 mM to about 650 mM, about 5 mM to about 600 mM, about 5 mM to about 550 mM, about 5 mM to about 500 mM, about 5 mM to about 450 mM, about 5 mM to about 400 mM, about 5 mM to about 350 mM, about 5 mM to about 300 mM, about 5 mM to about 290 mM, about 5 mM to about 280 mM, about 5 mM to about 270 mM, about 5 mM to about 260 mM, about 5 mM to about 250 mM, about 5 mM to about 240 mM, about 5 mM to about 230 mM, about 5 mM to about 220 mM, about 5 mM to about 210 mM, about 5 mM to about 200 mM, about 5 mM to about 190 mM, about 5 mM to about 180 mM, about 5 mM to about 170 mM, about 5 mM to about 160 mM, about 5 mM to about 150 mM, about 5 mM to about 140 mM, about 5 mM to about 130 mM, about 5 mM to about 120 mM, about 5 mM to about 110 mM, about 5 mM to about 100 mM, about 5 mM to about 95 mM, about 5 mM to about 90 mM, about 5 mM to about 85 mM, about 5 mM to about 80 mM, about 5 mM to about 75 mM, about 5 mM to about 70 mM, about 5 mM to about 65 mM, about 5 mM to about 60 mM, about 5 mM to about 55 mM, about 5 mM to about 50 mM, about 5 mM to about 45 mM, about 5 mM to about 40 mM, about 5 mM to about 35 mM, about 5 mM to about 30 mM, about 5 mM to about 25 mM, about 5 mM to about 20 mM, about 5 mM to about 15 mM, about 5 mM to about 10 mM, about 10 mM to about 750 mM, about 10 mM to about 700 mM, about 10 mM to about 650 mM, about 10 mM to about 600 mM, about 10 mM to about 550 mM, about 10 mM to about 500 mM, about 10 mM to about 450 mM, about 10 mM to about 400 mM, about 10 mM to about 350 mM, about 10 mM to about 300 mM, about 10 mM to about 290 mM, about 10 mM to about 280 mM, about 10 mM to about 270 mM, about 10 mM to about 260 mM, about 10 mM to about 250 mM, about 10 mM to about 240 mM, about 10 mM to about 230 mM, about 10 mM to about 220 mM, about 10 mM to about 210 mM, about 10 mM to about 200 mM, about 10 mM to about 190 mM, about 10 mM to about 180 mM, about 10 mM to about 170 mM, about 10 mM to about 160 mM, about 10 mM to about 150 mM, about 10 mM to about 140 mM, about 10 mM to about 130 mM, about 10 mM to about 120 mM, about 10 mM to about 110 mM, about 10 mM to about 100 mM, about 10 mM to about 95 mM, about 10 mM to about 90 mM, about 10 mM to about 85 mM, about 10 mM to about 80 mM, about 10 mM to about 75 mM, about 10 mM to about 70 mM, about 10 mM to about 65 mM, about 10 mM to about 60 mM, about 10 mM to about 55 mM, about 10 mM to about 50 mM, about 10 mM to about 45 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 15 mM to about 750 mM, about 15 mM to about 700 mM, about 15 mM to about 650 mM, about 15 mM to about 600 mM, about 15 mM to about 550 mM, about 15 mM to about 500 mM, about 15 mM to about 450 mM, about 15 mM to about 400 mM, about 15 mM to about 350 mM, about 15 mM to about 300 mM, about 15 mM to about 290 mM, about 15 mM to about 280 mM, about 15 mM to about 270 mM, about 15 mM to about 260 mM, about 15 mM to about 250 mM, about 15 mM to about 240 mM, about 15 mM to about 230 mM, about 15 mM to about 220 mM, about 15 mM to about 210 mM, about 15 mM to about 200 mM, about 15 mM to about 190 mM, about 15 mM to about 180 mM, about 15 mM to about 170 mM, about 15 mM to about 160 mM, about 15 mM to about 150 mM, about 15 mM to about 140 mM, about 15 mM to about 130 mM, about 15 mM to about 120 mM, about 15 mM to about 110 mM, about 15 mM to about 100 mM, about 15 mM to about 95 mM, about 15 mM to about 90 mM, about 15 mM to about 85 mM, about 15 mM to about 80 mM, about 15 mM to about 75 mM, about 15 mM to about 70 mM, about 15 mM to about 65 mM, about 15 mM to about 60 mM, about 15 mM to about 55 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, about 20 mM to about 750 mM, about 20 mM to about 700 mM, about 20 mM to about 650 mM, about 20 mM to about 600 mM, about 20 mM to about 550 mM, about 20 mM to about 500 mM, about 20 mM to about 450 mM, about 20 mM to about 400 mM, about 20 mM to about 350 mM, about 20 mM to about 300 mM, about 20 mM to about 290 mM, about 20 mM to about 280 mM, about 20 mM to about 270 mM, about 20 mM to about 260 mM, about 20 mM to about 250 mM, about 20 mM to about 240 mM, about 20 mM to about 230 mM, about 20 mM to about 220 mM, about 20 mM to about 210 mM, about 20 mM to about 200 mM, about 20 mM to about 190 mM, about 20 mM to about 180 mM, about 20 mM to about 170 mM, about 20 mM to about 160 mM, about 20 mM to about 150 mM, about 20 mM to about 140 mM, about 20 mM to about 130 mM, about 20 mM to about 120 mM, about 20 mM to about 110 mM, about 20 mM to about 100 mM, about 20 mM to about 95 mM, about 20 mM to about 90 mM, about 20 mM to about 85 mM, about 20 mM to about 80 mM, about 20 mM to about 75 mM, about 20 mM to about 70 mM, about 20 mM to about 65 mM, about 20 mM to about 60 mM, about 20 mM to about 55 mM, about 20 mM to about 50 mM, about 20 mM to about 45 mM, about 20 mM to about 40 mM, about 20 mM to about 35 mM, about 20 mM to about 30 mM, about 20 mM to about 25 mM, about 25 mM to about 750 mM, about 25 mM to about 700 mM, about 25 mM to about 650 mM, about 25 mM to about 600 mM, about 25 mM to about 550 mM, about 25 mM to about 500 mM, about 25 mM to about 450 mM, about 25 mM to about 400 mM, about 25 mM to about 350 mM, about 25 mM to about 300 mM, about 25 mM to about 290 mM, about 25 mM to about 280 mM, about 25 mM to about 270 mM, about 25 mM to about 260 mM, about 25 mM to about 250 mM, about 25 mM to about 240 mM, about 25 mM to about 230 mM, about 25 mM to about 220 mM, about 25 mM to about 210 mM, about 25 mM to about 200 mM, about 25 mM to about 190 mM, about 25 mM to about 180 mM, about 25 mM to about 170 mM, about 25 mM to about 160 mM, about 25 mM to about 150 mM, about 25 mM to about 140 mM, about 25 mM to about 130 mM, about 25 mM to about 120 mM, about 25 mM to about 110 mM, about 25 mM to about 100 mM, about 25 mM to about 95 mM, about 25 mM to about 90 mM, about 25 mM to about 85 mM, about 25 mM to about 80 mM, about 25 mM to about 75 mM, about 25 mM to about 70 mM, about 25 mM to about 65 mM, about 25 mM to about 60 mM, about 25 mM to about 50 mM, about 25 mM to about 45 mM, about 25 mM to about 40 mM, about 25 mM to about 35 mM, about 25 mM to about 30 mM, about 30 mM to about 750 mM, about 30 mM to about 700 mM, about 30 mM to about 650 mM, about 30 mM to about 600 mM, about 30 mM to about 550 mM, about 30 mM to about 500 mM, about 30 mM to about 450 mM, about 30 mM to about 400 mM, about 30 mM to about 350 mM, about 30 mM to about 300 mM, about 30 mM to about 290 mM, about 30 mM to about 280 mM, about 30 mM to about 270 mM, about 30 mM to about 260 mM, about 30 mM to about 250 mM, about 30 mM to about 240 mM, about 30 mM to about 230 mM, about 30 mM to about 220 mM, about 30 mM to about 210 mM, about 30 mM to about 200 mM, about 30 mM to about 190 mM, about 30 mM to about 180 mM, about 30 mM to about 170 mM, about 30 mM to about 160 mM, about 30 mM to about 150 mM, about 30 mM to about 140 mM, about 30 mM to about 130 mM, about 30 mM to about 120 mM, about 30 mM to about 110 mM, about 30 mM to about 100 mM, about 30 mM to about 95 mM, about 30 mM to about 90 mM, about 30 mM to about 85 mM, about 30 mM to about 80 mM, about 30 mM to about 75 mM, about 30 mM to about 70 mM, about 30 mM to about 65 mM, about 30 mM to about 60 mM, about 30 mM to about 55 mM, about 30 mM to about 50 mM, about 30 mM to about 45 mM, about 30 mM to about 40 mM, about 30 mM to about 35 mM, about 35 mM to about 750 mM, about 35 mM to about 700 mM, about 35 mM to about 650 mM, about 35 mM to about 600 mM, about 35 mM to about 550 mM, about 35 mM to about 500 mM, about 35 mM to about 450 mM, about 35 mM to about 400 mM, about 35 mM to about 350 mM, about 35 mM to about 300 mM, about 35 mM to about 290 mM, about 35 mM to about 280 mM, about 35 mM to about 270 mM, about 35 mM to about 260 mM, about 35 mM to about 250 mM, about 35 mM to about 240 mM, about 35 mM to about 230 mM, about 35 mM to about 220 mM, about 35 mM to about 210 mM, about 35 mM to about 200 mM, about 35 mM to about 190 mM, about 35 mM to about 180 mM, about 35 mM to about 170 mM, about 35 mM to about 160 mM, about 35 mM to about 150 mM, about 35 mM to about 140 mM, about 35 mM to about 130 mM, about 35 mM to about 120 mM, about 35 mM to about 110 mM, about 35 mM to about 100 mM, about 35 mM to about 95 mM, about 35 mM to about 90 mM, about 35 mM to about 85 mM<about 35 mM to about 80 mM, about 35 mM to about 75 mM, about 35 mM to about 70 mM, about 35 mM to about 65 mM, about 35 mM to about 60 mM, about 35 mM to about 55 mM, about 35 mM to about 50 mM, about 35 mM to about 45 mM, about 35 mM to about 40 mM, about 40 mM to about 750 mM, about 40 mM to about 700 mM, about 40 mM to about 650 mM, about 40 mM to about 600 mM, about 40 mM to about 550 mM, about 40 mM to about 500 mM, about 40 mM to about 450 mM, about 40 mM to about 400 mM, about 40 mM to about 350 mM, about 40 mM to about 300 mM, about 40 mM to about 290 mM, about 40 mM to about 280 mM, about 40 mM to about 270 mM, about 40 mM to about 260 mM, about 40 mM to about 250 mM, about 40 mM to about 240 mM, about 40 mM to about 230 mM, about 40 mM to about 220 mM, about 40 mM to about 210 mM, about 40 mM to about 200 mM, about 40 mM to about 190 mM, about 40 mM to about 180 mM, about 40 mM to about 170 mM, about 40 mM to about 160 mM, about 40 mM to about 150 mM, about 40 mM to about 140 mM, about 40 mM to about 130 mM, about 40 mM to about 120 mM, about 40 mM to about 110 mM, about 40 mM to about 100 mM, about 40 mM to about 95 mM, about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, about 40 mM to about 50 mM, about 40 mM to about 45 mM, about 45 mM to about 750 mM, about 45 mM to about 700 mM, about 45 mM to about 650 mM, about 45 mM to about 600 mM, about 45 mM to about 550 mM, about 45 mM to about 500 mM, about 45 mM to about 450 mM, about 45 mM to about 400 mM, about 45 mM to about 350 mM, about 45 mM to about 300 mM, about 45 mM to about 290 mM, about 45 mM to about 280 mM, about 45 mM to about 270 mM, about 45 mM to about 260 mM, about 45 mM to about 250 mM, about 45 mM to about 240 mM, about 45 mM to about 230 mM, about 45 mM to about 220 mM, about 45 mM to about 210 mM, about 45 mM to about 200 mM, about 45 mM to about 190 mM, about 45 mM to about 180 mM, about 45 mM to about 170 mM, about 45 mM to about 160 mM, about 45 mM to about 150 mM, about 45 mM to about 140 mM, about 45 mM to about 130 mM, about 45 mM to about 120 mM, about 45 mM to about 110 mM, about 45 mM to about 100 mM, about 45 mM to about 95 mM, about 45 mM to about 90 mM, about 45 mM to about 85 mM, about 45 mM to about 80 mM, about 45 mM to about 75 mM, about 45 mM to about 70 mM, about 45 mM to about 65 mM, about 45 mM to about 60 mM, about 45 mM to about 55 mM, about 45 mM to about 50 mM, about 50 mM to about 750 mM, about 50 mM to about 700 mM, about 50 mM to about 650 mM, about 50 mM to about 600 mM, about 50 mM to about 550 mM, about 50 mM to about 500 mM, about 50 mM to about 450 mM, about 50 mM to about 400 mM, about 50 mM to about 350 mM, about 50 mM to about 300 mM, about 50 mM to about 290 mM, about 50 mM to about 280 mM, about 50 mM to about 270 mM, about 50 mM to about 260 mM, about 50 mM to about 250 mM, about 50 mM to about 240 mM, about 50 mM to about 230 mM, about 50 mM to about 220 mM, about 50 mM to about 210 mM, about 50 mM to about 200 mM, about 50 mM to about 190 mM, about 50 mM to about 180 mM, about 50 mM to about 170 mM, about 50 mM to about 160 mM, about 50 mM to about 150 mM, about 50 mM to about 140 mM, about 50 mM to about 130 mM, about 50 mM to about 120 mM, about 50 mM to about 110 mM, about 50 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, about 60 mM to about 750 mM, about 60 mM to about 700 mM, about 60 mM to about 650 mM, about 60 mM to about 600 mM, about 60 mM to about 550 mM, about 60 mM to about 500 mM, about 60 mM to about 450 mM, about 60 mM to about 400 mM, about 60 mM to about 350 mM, about 60 mM to about 300 mM, about 60 mM to about 290 mM, about 60 mM to about 280 mM, about 60 mM to about 270 mM, about 60 mM to about 260 mM, about 60 mM to about 250 mM, about 60 mM to about 240 mM, about 60 mM to about 230 mM, about 60 mM to about 220 mM, about 60 mM to about 210 mM, about 60 mM to about 200 mM, about 60 mM to about 190 mM, about 60 mM to about 180 mM, about 60 mM to about 170 mM, about 60 mM to about 160 mM, about 60 mM to about 150 mM, about 60 mM to about 140 mM, about 60 mM to about 130 mM, about 60 mM to about 120 mM, about 60 mM to about 110 mM, about 60 mM to about 100 mM, about 60 mM to about 95 mM, about 60 mM to about 90 mM, about 60 mM to about 85 mM, about 60 mM to about 80 mM, about 60 mM to about 75 mM, about 60 mM to about 70 mM, about 70 mM to about 750 mM, about 70 mM to about 700 mM, about 70 mM to about 650 mM, about 70 mM to about 600 mM, about 70 mM to about 550 mM, about 70 mM to about 500 mM, about 70 mM to about 450 mM, about 70 mM to about 400 mM, about 70 mM to about 350 mM, about 70 mM to about 300 mM, about 70 mM to about 290 mM, about 70 mM to about 280 mM, about 70 mM to about 270 mM, about 70 mM to about 260 mM, about 70 mM to about 250 mM, about 70 mM to about 240 mM, about 70 mM to about 230 mM, about 70 mM to about 220 mM, about 70 mM to about 210 mM, about 70 mM to about 200 mM, about 70 mM to about 190 mM, about 70 mM to about 180 mM, about 70 mM to about 170 mM, about 70 mM to about 160 mM, about 70 mM to about 150 mM, about 70 mM to about 140 mM, about 70 mM to about 130 mM, about 70 mM to about 120 mM, about 70 mM to about 110 mM, about 70 mM to about 100 mM, about 70 mM to about 95 mM, about 70 mM to about 90 mM, about 70 mM to about 85 mM, about 70 mM to about 80 mM, about 80 mM to about 750 mM, about 80 mM to about 700 mM, about 80 mM to about 650 mM, about 80 mM to about 600 mM, about 80 mM to about 550 mM, about 80 mM to about 500 mM, about 80 mM to about 450 mM, about 80 mM to about 400 mM, about 80 mM to about 350 mM, about 80 mM to about 300 mM, about 80 mM to about 290 mM, about 80 mM to about 280 mM, about 80 mM to about 270 mM, about 80 mM to about 260 mM, about 80 mM to about 250 mM, about 80 mM to about 240 mM, about 80 mM to about 230 mM, about 80 mM to about 220 mM, about 80 mM to about 210 mM, about 80 mM to about 200 mM, about 80 mM to about 190 mM, about 80 mM to about 180 mM, about 80 mM to about 170 mM, about 80 mM to about 160 mM, about 80 mM to about 150 mM, about 80 mM to about 140 mM, about 80 mM to about 130 mM, about 80 mM to about 120 mM, about 80 mM to about 110 mM, about 80 mM to about 100 mM, about 80 mM to about 95 mM, about 80 mM to about 90 mM, about 90 mM to about 750 mM, about 90 mM to about 700 mM, about 90 mM to about 650 mM, about 90 mM to about 600 mM, about 90 mM to about 550 mM, about 90 mM to about 500 mM, about 90 mM to about 450 mM, about 90 mM to about 400 mM, about 90 mM to about 350 mM, about 90 mM to about 300 mM, about 90 mM to about 290 mM, about 90 mM to about 280 mM, about 90 mM to about 270 mM, about 90 mM to about 260 mM, about 90 mM to about 250 mM, about 90 mM to about 240 mM, about 90 mM to about 230 mM, about 90 mM to about 220 mM, about 90 mM to about 210 mM, about 90 mM to about 200 mM, about 90 mM to about 190 mM, about 90 mM to about 180 mM, about 90 mM to about 170 mM, about 90 mM to about 160 mM, about 90 mM to about 150 mM, about 90 mM to about 140 mM, about 90 mM to about 130 mM, about 90 mM to about 120 mM, about 90 mM to about 110 mM, about 90 mM to about 100 mM, about 100 mM to about 750 mM, about 100 mM to about 700 mM, about 100 mM to about 650 mM, about 100 mM to about 600 mM, about 100 mM to about 550 mM, about 100 mM to about 500 mM, about 100 mM to about 450 mM, about 100 mM to about 400 mM, about 100 mM to about 350 mM, about 100 mM to about 300 mM, about 100 mM to about 290 mM, about 100 mM to about 280 mM, about 100 mM to about 270 mM, about 100 mM to about 260 mM, about 100 mM to about 250 mM, about 100 mM to about 240 mM, about 100 mM to about 230 mM, about 100 mM to about 220 mM, about 100 mM to about 210 mM, about 100 mM to about 200 mM, about 100 mM to about 190 mM, about 100 mM to about 180 mM, about 100 mM to about 170 mM, about 100 mM to about 160 mM, about 100 mM to about 150 mM, about 100 mM to about 140 mM, about 100 mM to about 130 mM, about 100 mM to about 120 mM, about 100 mM to about 110 mM, about 110 mM to about 750 mM, about 110 mM to about 700 mM, about 110 mM to about 650 mM, about 110 mM to about 600 mM, about 110 mM to about 550 mM, about 110 mM to about 500 mM, about 110 mM to about 450 mM, about 110 mM to about 400 mM, about 110 mM to about 350 mM, about 110 mM to about 300 mM, about 110 mM to about 290 mM, about 110 mM to about 280 mM, about 110 mM to about 270 mM, about 110 mM to about 260 mM, about 110 mM to about 250 mM, about 110 mM to about 240 mM, about 110 mM to about 230 mM, about 110 mM to about 220 mM, about 110 mM to about 210 mM, about 110 mM to about 200 mM, about 110 mM to about 190 mM, about 110 mM to about 180 mM, about 110 mM to about 170 mM, about 110 mM to about 160 mM, about 110 mM to about 150 mM, about 110 mM to about 140 mM, about 110 mM to about 130 mM, about 110 mM to about 120 mM, about 120 mM to about 750 mM, about 120 mM to about 700 mM, about 120 mM to about 650 mM, about 120 mM to about 600 mM, about 120 mM to about 550 mM, about 120 mM to about 500 mM, about 120 mM to about 450 mM, about 120 mM to about 400 mM, about 120 mM to about 350 mM, about 120 mM to about 300 mM, about 120 mM to about 290 mM, about 120 mM to about 280 mM, about 120 mM to about 270 mM, about 120 mM to about 260 mM, about 120 mM to about 250 mM, about 120 mM to about 240 mM, about 120 mM to about 230 mM, about 120 mM to about 220 mM, about 120 mM to about 210 mM, about 120 mM to about 200 mM, about 120 mM to about 190 mM, about 120 mM to about 180 mM, about 120 mM to about 170 mM, about 120 mM to about 160 mM, about 120 mM to about 150 mM, about 120 mM to about 140 mM, about 120 mM to about 130 mM, about 130 mM to about 750 mM, about 130 mM to about 700 mM, about 130 mM to about 650 mM, about 130 mM to about 600 mM, about 130 mM to about 550 mM, about 130 mM to about 500 mM, about 130 mM to about 450 mM, about 130 mM to about 400 mM, about 130 mM to about 350 mM, about 130 mM to about 300 mM, about 130 mM to about 290 mM, about 130 mM to about 280 mM, about 130 mM to about 270 mM, about 130 mM to about 260 mM, about 130 mM to about 250 mM, about 130 mM to about 240 mM, about 130 mM to about 230 mM, about 130 mM to about 220 mM, about 130 mM to about 210 mM, about 130 mM to about 200 mM, about 130 mM to about 190 mM, about 130 mM to about 180 mM, about 130 mM to about 170 mM, about 130 mM to about 160 mM, about 130 mM to about 150 mM, about 130 mM to about 140 mM, about 140 mM to about 750 mM, about 140 mM to about 700 mM, about 140 mM to about 650 mM, about 140 mM to about 600 mM, about 140 mM to about 550 mM, about 140 mM to about 500 mM, about 140 mM to about 450 mM, about 140 mM to about 400 mM, about 140 mM to about 350 mM, about 140 mM to about 300 mM, about 140 mM to about 290 mM, about 140 mM to about 280 mM, about 140 mM to about 270 mM, about 140 mM to about 260 mM, about 140 mM to about 250 mM, about 140 mM to about 240 mM, about 140 mM to about 230 mM, about 140 mM to about 220 mM, about 140 mM to about 210 mM, about 140 mM to about 200 mM, about 140 mM to about 190 mM, about 140 mM to about 180 mM, about 140 mM to about 170 mM, about 140 mM to about 160 mM, about 140 mM to about 150 mM, about 150 mM to about 750 mM, about 150 mM to about 700 mM, about 150 mM to about 650 mM, about 150 mM to about 600 mM, about 150 mM to about 550 mM, about 150 mM to about 500 mM, about 150 mM to about 450 mM, about 150 mM to about 400 mM, about 150 mM to about 350 mM, about 150 mM to about 300 mM, about 150 mM to about 290 mM, about 150 mM to about 280 mM, about 150 mM to about 270 mM, about 150 mM to about 260 mM, about 150 mM to about 250 mM, about 150 mM to about 240 mM, about 150 mM to about 230 mM, about 150 mM to about 220 mM, about 150 mM to about 210 mM, about 150 mM to about 200 mM, about 150 mM to about 190 mM, about 150 mM to about 180 mM, about 150 mM to about 170 mM, about 150 mM to about 160 mM, about 160 mM to about 750 mM, about 160 mM to about 700 mM, about 160 mM to about 650 mM, about 160 mM to about 600 mM, about 160 mM to about 550 mM, about 160 mM to about 500 mM, about 160 mM to about 450 mM, about 160 mM to about 400 mM, about 160 mM to about 350 mM, about 160 mM to about 300 mM, about 160 mM to about 290 mM, about 160 mM to about 280 mM, about 160 mM to about 270 mM, about 160 mM to about 260 mM, about 160 mM to about 250 mM, about 160 mM to about 240 mM, about 160 mM to about 230 mM, about 160 mM to about 220 mM, about 160 mM to about 210 mM, about 160 mM to about 200 mM, about 160 mM to about 190 mM, about 160 mM to about 180 mM, about 160 mM to about 170 mM, about 170 mM to about 750 mM, about 170 mM to about 700 mM, about 170 mM to about 650 mM, about 170 mM to about 600 mM, about 170 mM to about 550 mM, about 170 mM to about 500 mM, about 170 mM to about 450 mM, about 170 mM to about 400 mM, about 170 mM to about 350 mM, about 170 mM to about 300 mM, about 170 mM to about 290 mM, about 170 mM to about 280 mM, about 170 mM to about 270 mM, about 170 mM to about 260 mM, about 170 mM to about 250 mM, about 170 mM to about 240 mM, about 170 mM to about 230 mM, about 170 mM to about 220 mM, about 170 mM to about 210 mM, about 170 mM to about 200 mM, about 170 mM to about 190 mM, about 170 mM to about 180 mM, about 180 mM to about 750 mM, about 180 mM to about 700 mM, about 180 mM to about 650 mM, about 180 mM to about 600 mM, about 180 mM to about 550 mM, about 180 mM to about 500 mM, about 180 mM to about 450 mM, about 180 mM to about 400 mM, about 180 mM to about 350 mM, about 180 mM to about 300 mM, about 180 mM to about 290 mM, about 180 mM to about 280 mM, about 180 mM to about 270 mM, about 180 mM to about 260 mM, about 180 mM to about 250 mM, about 180 mM to about 240 mM, about 180 mM to about 230 mM, about 180 mM to about 220 mM, about 180 mM to about 210 mM, about 180 mM to about 200 mM, about 180 mM to about 190 mM, about 190 mM to about 750 mM, about 190 mM to about 700 mM, about 190 mM to about 650 mM, about 190 mM to about 600 mM, about 190 mM to about 550 mM, about 190 mM to about 500 mM, about 190 mM to about 450 mM, about 190 mM to about 400 mM, about 190 mM to about 350 mM, about 190 mM to about 300 mM, about 190 mM to about 290 mM, about 190 mM to about 280 mM, about 190 mM to about 270 mM, about 190 mM to about 260 mM, about 190 mM to about 250 mM, about 190 mM to about 240 mM, about 190 mM to about 230 mM, about 190 mM to about 220 mM, about 190 mM to about 210 mM, about 190 mM to about 200 mM, about 200 mM to about 750 mM, about 200 mM to about 700 mM, about 200 mM to about 650 mM, about 200 mM to about 600 mM, about 200 mM to about 550 mM, about 200 mM to about 500 mM, about 200 mM to about 450 mM, about 200 mM to about 400 mM, about 200 mM to about 350 mM, about 200 mM to about 300 mM, about 200 mM to about 290 mM, about 200 mM to about 280 mM, about 200 mM to about 270 mM, about 200 mM to about 260 mM, about 200 mM to about 250 mM, about 200 mM to about 240 mM, about 200 mM to about 230 mM, about 200 mM to about 220 mM, about 200 mM to about 210 mM, about 210 mM to about 750 mM, about 210 mM to about 700 mM, about 210 mM to about 650 mM, about 210 mM to about 600 mM, about 210 mM to about 550 mM, about 210 mM to about 500 mM, about 210 mM to about 450 mM, about 210 mM to about 400 mM, about 210 mM to about 350 mM, about 210 mM to about 300 mM, about 210 mM to about 290 mM, about 210 mM to about 280 mM, about 210 mM to about 270 mM, about 210 mM to about 260 mM, about 210 mM to about 250 mM, about 210 mM to about 240 mM, about 210 mM to about 230 mM, about 210 mM to about 220 mM, about 220 mM to about 750 mM, about 220 mM to about 700 mM, about 220 mM to about 650 mM, about 220 mM to about 600 mM, about 220 mM to about 550 mM, about 220 mM to about 500 mM, about 220 mM to about 450 mM, about 220 mM to about 400 mM, about 220 mM to about 350 mM, about 220 mM to about 300 mM, about 220 mM to about 290 mM, about 220 mM to about 280 mM, about 220 mM to about 270 mM, about 220 mM to about 260 mM, about 220 mM to about 250 mM, about 220 mM to about 240 mM, about 220 mM to about 230 mM, about 230 mM to about 750 mM, about 230 mM to about 700 mM, about 230 mM to about 650 mM, about 230 mM to about 600 mM, about 230 mM to about 550 mM, about 230 mM to about 500 mM, about 230 mM to about 450 mM, about 230 mM to about 400 mM, about 230 mM to about 350 mM, about 230 mM to about 300 mM, about 230 mM to about 290 mM, about 230 mM to about 280 mM, about 230 mM to about 270 mM, about 230 mM to about 260 mM, about 230 mM to about 250 mM, about 230 mM to about 240 mM, about 240 mM to about 750 mM, about 240 mM to about 700 mM, about 240 mM to about 650 mM, about 240 mM to about 600 mM, about 240 mM to about 550 mM, about 240 mM to about 500 mM, about 240 mM to about 450 mM, about 240 mM to about 400 mM, about 240 mM to about 350 mM, about 240 mM to about 300 mM, about 240 mM to about 290 mM, about 240 mM to about 280 mM, about 240 mM to about 270 mM, about 240 mM to about 260 mM, about 240 mM to about 250 mM, about 250 mM to about 750 mM, about 250 mM to about 700 mM, about 250 mM to about 650 mM, about 250 mM to about 600 mM, about 250 mM to about 550 mM, about 250 mM to about 500 mM, about 250 mM to about 450 mM, about 250 mM to about 400 mM, about 250 mM to about 350 mM, about 250 mM to about 300 mM, about 250 mM to about 290 mM, about 250 mM to about 280 mM, about 250 mM to about 270 mM, about 250 mM to about 260 mM, about 260 mM to about 750 mM, about 260 mM to about 700 mM, about 260 mM to about 650 mM, about 260 mM to about 600 mM, about 260 mM to about 550 mM, about 260 mM to about 500 mM, about 260 mM to about 450 mM, about 260 mM to about 400 mM, about 260 mM to about 350 mM, about 260 mM to about 300 mM, about 260 mM to about 290 mM, about 260 mM to about 280 mM, about 260 mM to about 270 mM, about 270 mM to about 750 mM, about 270 mM to about 700 mM, about 270 mM to about 650 mM, about 270 mM to about 600 mM, about 270 mM to about 550 mM, about 270 mM to about 500 mM, about 270 mM to about 450 mM, about 270 mM to about 400 mM, about 270 mM to about 350 mM, about 270 mM to about 300 mM, about 270 mM to about 290 mM, about 270 mM to about 280 mM, about 280 mM to about 750 mM, about 280 mM to about 700 mM, about 280 mM to about 650 mM, about 280 mM to about 600 mM, about 280 mM to about 550 mM, about 280 mM to about 500 mM, about 280 mM to about 450 mM, about 280 mM to about 400 mM, about 280 mM to about 350 mM, about 280 mM to about 300 mM, about 280 mM to about 290 mM, about 290 mM to about 750 mM, about 290 mM to about 700 mM, about 290 mM to about 650 mM, about 290 mM to about 600 mM, about 290 mM to about 550 mM, about 290 mM to about 500 mM, about 290 mM to about 450 mM, about 290 mM to about 400 mM, about 290 mM to about 350 mM, about 290 mM to about 300 mM, about 300 mM to about 750 mM, about 300 mM to about 700 mM, about 300 mM to about 650 mM, about 300 mM to about 600 mM, about 300 mM to about 550 mM, about 300 mM to about 500 mM, about 300 mM to about 450 mM, about 300 mM to about 400 mM, about 300 mM to about 350 mM, about 350 mM to about 750 mM, about 350 mM to about 700 mM, about 350 mM to about 650 mM, about 350 mM to about 600 mM, about 350 mM to about 550 mM, about 350 mM to about 500 mM, about 350 mM to about 450 mM, about 350 mM to about 400 mM, about 400 mM to about 750 mM, about 400 mM to about 700 mM, about 400 mM to about 650 mM, about 400 mM to about 600 mM, about 400 mM to about 550 mM, about 400 mM to about 500 mM, about 400 mM to about 450 mM, about 450 mM to about 750 mM, about 450 mM to about 700 mM, about 450 mM to about 650 mM, about 450 mM to about 600 mM, about 450 mM to about 550 mM, about 450 mM to about 500 mM, about 500 mM to about 750 mM, about 500 mM to about 700 mM, about 500 mM to about 650 mM, about 500 mM to about 600 mM, about 500 mM to about 550 mM, about 550 mM to about 750 mM, about 550 mM to about 700 mM, about 550 mM to about 650 mM, about 550 mM to about 600 mM, about 600 mM to about 750 mM, about 600 mM to about 700 mM, about 600 mM to about 650 mM, about 650 mM to about 750 mM, about 650 mM to about 700 mM, or about 700 to about 750 mM.

pH

Any of the aqueous antibody formulations described herein can have a pH of about 4 to about 8, about 4 to about 7.8, about 4 to about 7.6, about 4 to about 7.4, about 4 to about 7.2, about 4 to about 7, about 4 to about 6.8, about 4 to about 6.6, about 4 to about 6.4, about 4 to about 6.2, about 4 to about 6, about 4 to about 5.8, about 4 to about 5.6, about 4 to about 5.4, about 4 to about 5.2, about 4 to about 5, about 4 to about 4.8, about 4 to about 4.6, about 4 to about 4.4, about 4 to about 4.2, about 4.2 to about 8, about 4.2 to about 7.8, about 4.2 to about 7.6, about 4.2 to about 7.4, about 4.2 to about 7.2, about 4.2 to about 7, about 4.2 to about 6.8, about 4.2 to about 6.6, about 4.2 to about 6.4, about 4.2 to about 6.2, about 4.2 to about 6, about 4.2 to about 5.8, about 4.2 to about 5.6, about 4.2 to about 5.4, about 4.2 to about 5.2, about 4.2 to about 5, about 4.2 to about 4.8, about 4.2 to about 4.6, about 4.2 to about 4.4, about 4.4 to about 8, about 4.4 to about 7.8, about 4.4 to about 7.6, about 4.4 to about 7.4, about 4.4 to about 7.2, about 4.4 to about 7, about 4.4 to about 6.8, about 4.4 to about 6.6, about 4.4 to about 6.4, about 4.4 to about 6.2, about 4.4 to about 6, about 4.4 to about 5.8, about 4.4 to about 5.6, about 4.4 to about 5.4, about 4.4 to about 5.2, about 4.4 to about 5, about 4.4 to about 4.8, about 4.4 to about 4.6, about 4.6 to about 8, about 4.6 to about 7.8, about 4.6 to about 7.6, about 4.6 to about 7.4, about 4.6 to about 7.2, about 4.6 to about 7, about 4.6 to about 6.8, about 4.6 to about 6.6, about 4.6 to about 6.4, about 4.6 to about 6.2, about 4.6 to about 6, about 4.6 to about 5.8, about 4.6 to about 5.6, about 4.6 to about 5.4, about 4.6 to about 5.2, about 4.6 to about 5, about 4.6 to about 4.8, about 4.8 to about 8, about 4.8 to about 7.8, about 4.8 to about 7.6, about 4.8 to about 7.4, about 4.8 to about 7.2, about 4.8 to about 7, about 4.8 to about 6.8, about 4.8 to about 6.6, about 4.8 to about 6.4, about 4.8 to about 6.2, about 4.8 to about 6, about 4.8 to about 5.8, about 4.8 to about 5.6, about 4.8 to about 5.4, about 4.8 to about 5.2, about 4.8 to about 5, about 5 to about 8, about 5 to about 7.8, about 5 to about 7.6, about 5 to about 7.4, about 5 to about 7.4, about 5 to about 7.2, about 5 to about 7, about 5 to about 6.8, about 5 to about 6.6, about 5 to about 6.4, about 5 to about 6.2, about 5 to about 6, about 5 to about 5.8, about 5 to about 5.6, about 5 to about 5.4, about 5 to about 5.2, about 5.2 to about 8, about 5.2 to about 7.8, about 5.2 to about 7.6, about 5.2 to about 7.4, about 5.2 to about 7.2, about 5.2 to about 7, about 5.2 to about 6.8, about 5.2 to about 6.6, about 5.2 to about 6.4, about 5.2 to about 6.2, about 5.2 to about 6, about 5.2 to about 5.8, about 5.2 to about 5.6, about 5.2 to about 5.4, about 5.4 to about 8, about 5.4 to about 7.8, about 5.4 to about 7.6, about 5.4 to about 7.4, about 5.4 to about 7.2, about 5.4 to about 7, about 5.4 to about 6.8, about 5.4 to about 6.6, about 5.4 to about 6.4, about 5.4 to about 6.2, about 5.4 to about 6, about 5.4 to about 5.8, about 5.4 to about 5.6, about 5.6 to about 8, about 5.6 to about 7.8, about 5.6 to about 7.6, about 5.6 to about 7.4, about 5.6 to about 7.2, about 5.6 to about 7, about 5.6 to about 6.8, about 5.6 to about 6.6, about 5.6 to about 6.4, about 5.6 to about 6.2, about 5.6 to about 6, about 5.6 to about 5.8, about 5.8 to about 8, about 5.8 to about 7.8, about 5.8 to about 7.6, about 5.8 to about 7.4, about 5.8 to about 7.2, about 5.8 to about 7, about 5.8 to about 6.8, about 5.8 to about 6.6, about 5.8 to about 6.4, about 5.8 to about 6.2, about 5.8 to about 6, about 6 to about 8, about 6 to about 7.8, about 6 to about 7.6, about 6 to about 7.4, about 6 to about 7.2, about 6 to about 7, about 6 to about 6.8, about 6 to about 6.6, about 6 to about 6.4, about 6 to about 6.2, about 6.2 to about 8, about 6.2 to about 7.8, about 6.2 to about 7.6, about 6.2 to about 7.4, about 6.2 to about 7.2, about 6.2 to about 7, about 6.2 to about 6.8, about 6.2 to about 6.6, about 6.2 to about 6.4, about 6.4 to about 8, about 6.4 to about 7.6, about 6.4 to about 7.4, about 6.4 to about 7.2, about 6.4 to about 7, about 6.4 to about 6.8, about 6.4 to about 6.6, about 6.6 to about 8, about 6.6 to about 7.8, about 6.6 to about 7.6, about 6.6 to about 7.4, about 6.6 to about 7.2, about 6.6 to about 7, about 6.6 to about 6.8, about 6.8 to about 8, about 6.8 to about 7.8, about 6.8 to about 7.6, about 6.8 to about 7.4, about 6.8 to about 7.2, about 6.8 to about 7, about 7 to about 8, about 7 to about 7.8, about 7 to about 7.6, about 7 to about 7.4, about 7 to about 7.2, about 7.2 to about 8, about 7.2 to about 7.8, about 7.2 to about 7.6, about 7.2 to about 7.4, about 7.4 to about 8, about 7.4 to about 7.8, about 7.4 to about 7.6, about 7.6 to about 8, about 7.6 to about 7.8, or about 7.8 to about 8.

### Formulation Stability

In some embodiments of any of the aqueous antibody formulations described herein, the formulation is a stable formulation. A “stable” formulation is one in which a protein of interest (e.g., an antibody or an antigen-binding antibody fragment) therein essentially retains its physical stability and/or chemical stability and/or biological activity upon storage at about 4° C. to about 25° C. Various analytical techniques for measuring protein stability are known in the art. See, e.g., Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Additional methods for determining the stability of a protein (e.g., an antibody or antigen-binding antibody fragment) in a formulation are described in the Examples section. In some examples, the stability of a protein (e.g., an antibody or an antigen-binding antibody fragment) is determined according to the percentage of monomer protein in the solution, with a low percentage of degraded (e.g., fragmented) and/or aggregated protein. For example, an aqueous formulation comprising a stable protein may include at least 95% monomer protein. Alternatively, an aqueous formulation of the invention may include no more than 5% (e.g., no more than 4.5%, no more than 4.0%, no more than 3.5%, no more than 3.0%, no more than 2.5%, no more than 2.0%, no more than 1.5%, no more than 1.0%, or no more than 0.5%) aggregates and/or degraded protein.

In some embodiments of any of the aqueous antibody formulations described herein, the formulation has improved stability as compared to a control antibody or antigen-binding antibody fragment (e.g., as compared a control antibody formulation that includes all of the same components, except it does not include any of the following salts: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate).

In some embodiments of any of the aqueous antibody formulations described herein, the formulation is stable (e.g., % HMW by SEC ≤5%) at 25° C. for 1 hour to about 2 years. In some embodiments of any of the aqueous antibody formulations described herein, the formulation is stable (e.g., % HMW by SEC ≤5%) at 25° C. for about 1 hour to about 2 years (e.g., about 1 hour to about 24 months, about 1 hour to about 22 months, about 1 hour to about 20 months, about 1 hour to about 18 months, about 1 hour to about 16 months, about 1 hour to about 14 months, about 1 hour to about 12 months, about 1 hour to about 10 months, about 1 hour to about 8 months, about 1 hour to about 6 months, about 1 hour to about 4 months, about 1 hour to about 2 months, about 1 hour to about 1 month, about 1 hour to about 3 weeks, about 1 hour to about 2 weeks, about 1 hour to about 1 week, about 1 hour to about 6 days, about 1 hour to about 4 days, about 1 hour to about 2 days, about 1 hour to about 1 day, about 1 hour to about 28 days, about 1 hour to about 26 days, about 1 hour to about 24 days, about 1 hour to about 22 days, about 1 hour to about 20 days, about 1 hour to about 18 days, about 1 hour to about 16 days, about 1 hour to about 14 days, about 1 hour to about 12 days, about 1 hour to about 10 days, about 1 hour to about 8 days, about 1 hour to about 7 days, about 1 hour to about 6 days, about 1 hour to about 5 days, about 1 hour to about 4 days, about 1 hour to about 3 days, about 1 hour to about 2 days, about 1 hour to about 1 day, about 1 hour to about 22 hours, about 1 hour to about 20 hours, about 1 hour to about 18 hours, about 1 hour to about 16 hours, about 1 hour to about 14 hours, about 1 hour to about 12 hours, about 1 hour to about 10 hours, about 1 hour to about 8 hours, about 1 hour to about 6 hours, about 1 hour to about 4 hours, about 1 hour to about 2 hours, about 2 hours to about 24 months, about 2 hours to about 22 months, about 2 hours to about 20 months, about 2 hours to about 18 months, about 2 hours to about 16 months, about 2 hours to about 14 months, about 2 hours to about 12 months, about 2 hours to about 10 months, about 2 hours to about 8 months, about 2 hours to about 6 months, about 2 hours to about 4 months, about 2 hours to about 2 months, about 2 hours to about 1 month, about 2 hours to about 3 weeks, about 2 hours to about 2 weeks, about 2 hours to about 1 week, about 2 hours to about 6 days, about 2 hours to about 4 days, about 2 hours to about 2 days, about 2 hours to about 1 day, about 2 hours to about 28 days, about 2 hours to about 26 days, about 2 hours to about 24 days, about 2 hours to about 22 days, about 2 hours to about 20 days, about 2 hours to about 18 days, about 2 hours to about 16 days, about 2 hours to about 14 days, about 2 hours to about 12 days, about 2 hours to about 10 days, about 2 hours to about 8 days, about 2 hours to about 7 days, about 2 hours to about 6 days, about 2 hours to about 5 days, about 2 hours to about 4 days, about 2 hours to about 3 days, about 2 hours to about 2 days, about 2 hours to about 1 day, about 2 hours to about 22 hours, about 2 hours to about 20 hours, about 2 hours to about 18 hours, about 2 hours to about 16 hours, about 2 hours to about 14 hours, about 2 hours to about 12 hours, about 2 hours to about 10 hours, about 2 hours to about 8 hours, about 2 hours to about 6 hours, about 2 hours to about 4 hours, about 4 hours to about 24 months, about 4 hours to about 22 months, about 4 hours to about 20 months, about 4 hours to about 18 months, about 4 hours to about 16 months, about 4 hours to about 14 months, about 4 hours to about 12 months, about 4 hours to about 10 months, about 4 hours to about 8 months, about 4 hours to about 6 months, about 4 hours to about 4 months, about 4 hours to about 2 months, about 4 hours to about 1 month, about 4 hours to about 3 weeks, about 4 hours to about 2 weeks, about 4 hours to about 1 week, about 4 hours to about 6 days, about 4 hours to about 4 days, about 4 hours to about 2 days, about 4 hours to about 1 day, about 4 hours to about 28 days, about 4 hours to about 26 days, about 4 hours to about 24 days, about 4 hours to about 22 days, about 4 hours to about 20 days, about 4 hours to about 18 days, about 4 hours to about 16 days, about 4 hours to about 14 days, about 4 hours to about 12 days, about 4 hours to about 10 days, about 4 hours to about 8 days, about 4 hours to about 7 days, about 4 hours to about 6 days, about 4 hours to about 5 days, about 4 hours to about 4 days, about 4 hours to about 3 days, about 4 hours to about 2 days, about 4 hours to about 1 day, about 4 hours to about 22 hours, about 4 hours to about 20 hours, about 4 hours to about 18 hours, about 4 hours to about 16 hours, about 4 hours to about 14 hours, about 4 hours to about 12 hours, about 4 hours to about 10 hours, about 4 hours to about 8 hours, about 4 hours to about 6 hours, about 6 hours to about 24 months, about 6 hours to about 22 months, about 6 hours to about 20 months, about 6 hours to about 18 months, about 6 hours to about 16 months, about 6 hours to about 14 months, about 6 hours to about 12 months, about 6 hours to about 10 months, about 6 hours to about 8 months, about 6 hours to about 6 months, about 6 hours to about 4 months, about 6 hours to about 2 months, about 6 hours to about 1 month, about 6 hours to about 3 weeks, about 6 hours to about 2 weeks, about 6 hours to about 1 week, about 6 hours to about 6 days, about 6 hours to about 4 days, about 6 hours to about 2 days, about 6 hours to about 1 day, about 6 hours to about 28 days, about 6 hours to about 26 days, about 6 hours to about 24 days, about 6 hours to about 22 days, about 6 hours to about 20 days, about 6 hours to about 18 days, about 6 hours to about 16 days, about 6 hours to about 14 days, about 6 hours to about 12 days, about 6 hours to about 10 days, about 6 hours to about 8 days, about 6 hours to about 7 days, about 6 hours to about 6 days, about 6 hours to about 5 days, about 6 hours to about 4 days, about 6 hours to about 3 days, about 6 hours to about 2 days, about 6 hours to about 1 day, about 6 hours to about 22 hours, about 6 hours to about 20 hours, about 6 hours to about 18 hours, about 6 hours to about 16 hours, about 6 hours to about 14 hours, about 6 hours to about 12 hours, about 6 hours to about 10 hours, about 6 hours to about 8 hours, about 8 hours to about 24 months, about 8 hours to about 22 months, about 8 hours to about 20 months, about 8 hours to about 18 months, about 8 hours to about 16 months, about 8 hours to about 14 months, about 8 hours to about 12 months, about 8 hours to about 10 months, about 8 hours to about 8 months, about 8 hours to about 6 months, about 8 hours to about 4 months, about 8 hours to about 2 months, about 8 hours to about 1 month, about 8 hours to about 3 weeks, about 8 hours to about 2 weeks, about 8 hours to about 1 week, about 8 hours to about 6 days, about 8 hours to about 4 days, about 8 hours to about 2 days, about 8 hours to about 1 day, about 8 hours to about 28 days, about 8 hours to about 26 days, about 8 hours to about 24 days, about 8 hours to about 22 days, about 8 hours to about 20 days, about 8 hours to about 18 days, about 8 hours to about 16 days, about 8 hours to about 14 days, about 8 hours to about 12 days, about 8 hours to about 10 days, about 8 hours to about 8 days, about 8 hours to about 7 days, about 8 hours to about 6 days, about 8 hours to about 5 days, about 8 hours to about 4 days, about 8 hours to about 3 days, about 8 hours to about 2 days, about 8 hours to about 1 day, about 8 hours to about 22 hours, about 8 hours to about 20 hours, about 8 hours to about 18 hours, about 8 hours to about 16 hours, about 8 hours to about 14 hours, about 8 hours to about 12 hours, about 8 hours to about 10 hours, about 10 hours to about 24 months, about 10 hours to about 22 months, about 10 hours to about 20 months, about 10 hours to about 18 months, about 10 hours to about 16 months, about 10 hours to about 14 months, about 10 hours to about 12 months, about 10 hours to about 10 months, about 10 hours to about 8 months, about 10 hours to about 6 months, about 10 hours to about 4 months, about 10 hours to about 2 months, about 10 hours to about 1 month, about 10 hours to about 3 weeks, about 10 hours to about 2 weeks, about 10 hours to about 1 week, about 10 hours to about 6 days, about 10 hours to about 4 days, about 10 hours to about 2 days, about 10 hours to about 1 day, about 10 hours to about 28 days, about 10 hours to about 26 days, about 10 hours to about 24 days, about 10 hours to about 22 days, about 10 hours to about 20 days, about 10 hours to about 18 days, about 10 hours to about 16 days, about 10 hours to about 14 days, about 10 hours to about 12 days, about 10 hours to about 10 days, about 10 hours to about 8 days, about 10 hours to about 7 days, about 10 hours to about 6 days, about 10 hours to about 5 days, about 10 hours to about 4 days, about 10 hours to about 3 days, about 10 hours to about 2 days, about 10 hours to about 1 day, about 10 hours to about 22 hours, about 10 hours to about 20 hours, about 10 hours to about 18 hours, about 10 hours to about 16 hours, about 10 hours to about 14 hours, about 10 hours to about 12 hours, about 12 hours to about 24 months, about 12 hours to about 22 months, about 12 hours to about 20 months, about 12 hours to about 18 months, about 12 hours to about 16 months, about 12 hours to about 14 months, about 12 hours to about 12 months, about 12 hours to about 10 months, about 12 hours to about 8 months, about 12 hours to about 6 months, about 12 hours to about 4 months, about 12 hours to about 2 months, about 12 hours to about 1 month, about 12 hours to about 3 weeks, about 12 hours to about 2 weeks, about 12 hours to about 1 week, about 12 hours to about 6 days, about 12 hours to about 4 days, about 12 hours to about 2 days, about 12 hours to about 1 day, about 12 hours to about 28 days, about 12 hours to about 26 days, about 12 hours to about 24 days, about 12 hours to about 22 days, about 12 hours to about 20 days, about 12 hours to about 18 days, about 12 hours to about 16 days, about 12 hours to about 14 days, about 12 hours to about 12 days, about 12 hours to about 10 days, about 12 hours to about 8 days, about 12 hours to about 7 days, about 12 hours to about 6 days, about 12 hours to about 5 days, about 12 hours to about 4 days, about 12 hours to about 3 days, about 12 hours to about 2 days, about 12 hours to about 1 day, about 12 hours to about 22 hours, about 12 hours to about 20 hours, about 12 hours to about 18 hours, about 12 hours to about 16 hours, about 12 hours to about 14 hours, about 14 hours to about 24 months, about 14 hours to about 22 months, about 14 hours to about 20 months, about 14 hours to about 18 months, about 14 hours to about 16 months, about 14 hours to about 14 months, about 14 hours to about 12 months, about 14 hours to about 10 months, about 14 hours to about 8 months, about 14 hours to about 6 months, about 14 hours to about 4 months, about 14 hours to about 2 months, about 14 hours to about 1 month, about 14 hours to about 3 weeks, about 14 hours to about 2 weeks, about 14 hours to about 1 week, about 14 hours to about 6 days, about 14 hours to about 4 days, about 14 hours to about 2 days, about 14 hours to about 1 day, about 14 hours to about 28 days, about 14 hours to about 26 days, about 14 hours to about 24 days, about 14 hours to about 22 days, about 14 hours to about 20 days, about 14 hours to about 18 days, about 14 hours to about 16 days, about 14 hours to about 14 days, about 14 hours to about 12 days, about 14 hours to about 10 days, about 14 hours to about 8 days, about 14 hours to about 7 days, about 14 hours to about 6 days, about 14 hours to about 5 days, about 14 hours to about 4 days, about 14 hours to about 3 days, about 14 hours to about 2 days, about 14 hours to about 1 day, about 14 hours to about 22 hours, about 14 hours to about 20 hours, about 14 hours to about 18 hours, about 16 hours to about 24 months, about 16 hours to about 22 months, about 16 hours to about 20 months, about 16 hours to about 18 months, about 16 hours to about 16 months, about 16 hours to about 14 months, about 16 hours to about 12 months, about 16 hours to about 10 months, about 16 hours to about 8 months, about 16 hours to about 6 months, about 16 hours to about 4 months, about 16 hours to about 2 months, about 16 hours to about 1 month, about 16 hours to about 3 weeks, about 16 hours to about 2 weeks, about 16 hours to about 1 week, about 16 hours to about 6 days, about 16 hours to about 4 days, about 16 hours to about 2 days, about 16 hours to about 1 day, about 16 hours to about 28 days, about 16 hours to about 26 days, about 16 hours to about 24 days, about 16 hours to about 22 days, about 16 hours to about 20 days, about 16 hours to about 18 days, about 16 hours to about 16 days, about 16 hours to about 14 days, about 16 hours to about 12 days, about 16 hours to about 10 days, about 16 hours to about 8 days, about 16 hours to about 7 days, about 16 hours to about 6 days, about 16 hours to about 5 days, about 16 hours to about 4 days, about 16 hours to about 3 days, about 16 hours to about 2 days, about 16 hours to about 1 day, about 16 hours to about 22 hours, about 16 hours to about 20 hours, about 18 hours to about 24 months, about 18 hours to about 22 months, about 18 hours to about 20 months, about 18 hours to about 18 months, about 18 hours to about 16 months, about 18 hours to about 14 months, about 18 hours to about 12 months, about 18 hours to about 10 months, about 18 hours to about 8 months, about 18 hours to about 6 months, about 18 hours to about 4 months, about 18 hours to about 2 months, about 18 hours to about 1 month, about 18 hours to about 3 weeks, about 18 hours to about 2 weeks, about 18 hours to about 1 week, about 18 hours to about 6 days, about 18 hours to about 4 days, about 18 hours to about 2 days, about 18 hours to about 1 day, about 18 hours to about 28 days, about 18 hours to about 26 days, about 18 hours to about 24 days, about 18 hours to about 22 days, about 18 hours to about 20 days, about 18 hours to about 18 days, about 18 hours to about 16 days, about 18 hours to about 14 days, about 18 hours to about 12 days, about 18 hours to about 10 days, about 18 hours to about 8 days, about 18 hours to about 7 days, about 18 hours to about 6 days, about 18 hours to about 5 days, about 18 hours to about 4 days, about 18 hours to about 3 days, about 18 hours to about 2 days, about 18 hours to about 1 day, about 18 hours to about 22 hours, about 18 hours to about 20 hours, about 20 hours to about 24 months, about 20 hours to about 22 months, about 20 hours to about 20 months, about 20 hours to about 18 months, about 20 hours to about 16 months, about 20 hours to about 14 months, about 20 hours to about 12 months, about 20 hours to about 10 months, about 20 hours to about 8 months, about 20 hours to about 6 months, about 20 hours to about 4 months, about 20 hours to about 2 months, about 20 hours to about 1 month, about 20 hours to about 3 weeks, about 20 hours to about 2 weeks, about 20 hours to about 1 week, about 20 hours to about 6 days, about 20 hours to about 4 days, about 20 hours to about 2 days, about 20 hours to about 1 day, about 20 hours to about 28 days, about 20 hours to about 26 days, about 20 hours to about 24 days, about 20 hours to about 22 days, about 20 hours to about 20 days, about 20 hours to about 18 days, about 20 hours to about 16 days, about 20 hours to about 14 days, about 20 hours to about 12 days, about 20 hours to about 10 days, about 20 hours to about 8 days, about 20 hours to about 7 days, about 20 hours to about 6 days, about 20 hours to about 5 days, about 20 hours to about 4 days, about 20 hours to about 3 days, about 20 hours to about 2 days, about 20 hours to about 1 day, about 20 hours to about 22 hours, about 1 day to about 24 months, about 1 day to about 22 months, about 1 day to about 20 months, about 1 day to about 18 months, about 1 day to about 16 months, about 1 day to about 14 months, about 1 day to about 12 months, about 1 day to about 10 months, about 1 day to about 8 months, about 1 day to about 6 months, about 1 day to about 4 months, about 1 day to about 2 months, about 1 day to about 1 month, about 1 day to about 3 weeks, about 1 day to about 2 weeks, about 1 day to about 1 week, about 1 day to about 6 days, about 1 day to about 4 days, about 1 day to about 2 days, about 1 day to about 1 day, about 1 day to about 28 days, about 1 day to about 26 days, about 1 day to about 24 days, about 1 day to about 22 days, about 1 day to about 20 days, about 1 day to about 18 days, about 1 day to about 16 days, about 1 day to about 14 days, about 1 day to about 12 days, about 1 day to about 10 days, about 1 day to about 8 days, about 1 day to about 7 days, about 1 day to about 6 days, about 1 day to about 5 days, about 1 day to about 4 days, about 1 day to about 3 days, about 1 day to about 2 days, about 2 days to about 24 months, about 2 days to about 22 months, about 2 days to about 20 months, about 2 days to about 18 months, about 2 days to about 16 months, about 2 days to about 14 months, about 2 days to about 12 months, about 2 days to about 10 months, about 2 days to about 8 months, about 2 days to about 6 months, about 2 days to about 4 months, about 2 days to about 2 months, about 2 days to about 1 month, about 2 days to about 3 weeks, about 2 days to about 2 weeks, about 2 days to about 1 week, about 2 days to about 6 days, about 2 days to about 4 days, about 2 days to about 2 days, about 2 days to about 2 days, about 2 days to about 28 days, about 2 days to about 26 days, about 2 days to about 24 days, about 2 days to about 22 days, about 2 days to about 20 days, about 2 days to about 18 days, about 2 days to about 16 days, about 2 days to about 14 days, about 2 days to about 12 days, about 2 days to about 10 days, about 2 days to about 8 days, about 2 days to about 7 days, about 2 days to about 6 days, about 2 days to about 5 days, about 2 days to about 4 days, about 2 days to about 3 days, about 4 days to about 24 months, about 4 days to about 22 months, about 4 days to about 20 months, about 4 days to about 18 months, about 4 days to about 16 months, about 4 days to about 14 months, about 4 days to about 12 months, about 4 days to about 10 months, about 4 days to about 8 months, about 4 days to about 6 months, about 4 days to about 4 months, about 4 days to about 2 months, about 4 days to about 1 month, about 4 days to about 3 weeks, about 4 days to about 2 weeks, about 4 days to about 1 week, about 4 days to about 6 days, about 4 days to about 4 days, about 4 days to about 2 days, about 4 days to about 4 days, about 4 days to about 28 days, about 4 days to about 26 days, about 4 days to about 24 days, about 4 days to about 22 days, about 4 days to about 20 days, about 4 days to about 18 days, about 4 days to about 16 days, about 4 days to about 14 days, about 4 days to about 12 days, about 4 days to about 10 days, about 4 days to about 8 days, about 4 days to about 7 days, about 4 days to about 6 days, about 4 days to about 5 days, about 7 days to about 24 months, about 7 days to about 22 months, about 7 days to about 20 months, about 7 days to about 18 months, about 7 days to about 16 months, about 7 days to about 14 months, about 7 days to about 12 months, about 7 days to about 10 months, about 7 days to about 8 months, about 7 days to about 6 months, about 7 days to about 4 months, about 7 days to about 2 months, about 7 days to about 1 month, about 7 days to about 3 weeks, about 7 days to about 2 weeks, about 7 days to about 1 week, about 7 days to about 6 days, about 7 days to about 4 days, about 7 days to about 2 days, about 7 days to about 7 days, about 7 days to about 28 days, about 7 days to about 26 days, about 7 days to about 24 days, about 7 days to about 22 days, about 7 days to about 20 days, about 7 days to about 18 days, about 7 days to about 16 days, about 7 days to about 14 days, about 7 days to about 12 days, about 7 days to about 10 days, about 7 days to about 8 days, about 1 week to about 24 months, about 1 week to about 22 months, about 1 week to about 20 months, about 1 week to about 18 months, about 1 week to about 16 months, about 1 week to about 14 months, about 1 week to about 12 months, about 1 week to about 10 months, about 1 week to about 8 months, about 1 week to about 6 months, about 1 week to about 4 months, about 1 week to about 2 months, about 1 week to about 1 month, about 1 week to about 3 weeks, about 1 week to about 2 weeks, about 2 weeks to about 24 months, about 2 weeks to about 22 months, about 2 weeks to about 20 months, about 2 weeks to about 18 months, about 2 weeks to about 16 months, about 2 weeks to about 14 months, about 2 weeks to about 12 months, about 2 weeks to about 10 months, about 2 weeks to about 8 months, about 2 weeks to about 6 months, about 2 weeks to about 4 months, about 2 weeks to about 2 months, about 2 weeks to about 1 month, about 2 weeks to about 3 weeks, about 3 weeks to about 24 months, about 3 weeks to about 22 months, about 3 weeks to about 20 months, about 3 weeks to about 18 months, about 3 weeks to about 16 months, about 3 weeks to about 14 months, about 3 weeks to about 12 months, about 3 weeks to about 10 months, about 3 weeks to about 8 months, about 3 weeks to about 6 months, about 3 weeks to about 4 months, about 3 weeks to about 2 months, about 3 weeks to about 1 month, about 1 month to about 24 months, about 1 month to about 22 months, about 1 month to about 20 months, about 1 month to about 18 months, about 1 month to about 16 months, about 1 month to about 14 months, about 1 month to about 12 months, about 1 month to about 10 months, about 1 month to about 8 months, about 1 month to about 6 months, about 1 month to about 4 months, about 1 month to about 2 months, about 2 months to about 24 months, about 2 months to about 22 months, about 2 months to about 20 months, about 2 months to about 18 months, about 2 months to about 16 months, about 2 months to about 14 months, about 2 months to about 12 months, about 2 months to about 10 months, about 2 months to about 8 months, about 2 months to about 6 months, about 2 months to about 4 months, about 4 months to about 24 months, about 4 months to about 22 months, about 4 months to about 20 months, about 4 months to about 18 months, about 4 months to about 16 months, about 4 months to about 14 months, about 4 months to about 12 months, about 4 months to about 10 months, about 4 months to about 8 months, about 4 months to about 6 months, about 6 months to about 24 months, about 6 months to about 22 months, about 6 months to about 20 months, about 6 months to about 18 months, about 6 months to about 16 months, about 6 months to about 14 months, about 6 months to about 12 months, about 6 months to about 10 months, about 6 months to about 8 months, about 10 months to about 24 months, about 10 months to about 22 months, about 10 months to about 20 months, about 10 months to about 18 months, about 10 months to about 16 months, about 10 months to about 14 months, about 10 months to about 12 months, about 12 months to about 24 months, about 12 months to about 22 months, about 12 months to about 20 months, about 12 months to about 18 months, about 12 months to about 16 months, about 12 months to about 14 months, about 14 months to about 24 months, about 14 months to about 22 months, about 14 months to about 20 months, about 14 months to about 18 months, about 14 months to about 16 months, about 16 months to about 24 months, about 16 months to about 22 months, about 16 months to about 20 months, about 16 months to about 18 months, about 18 months to about 24 months, about 18 months to about 22 months, about 18 months to about 20 months, about 20 months to about 22 months, about 20 months to about 24 months, or about 22 months to about 24 months (e.g., as determined using high-performance size exclusion chromatography).

In some embodiments of any of the aqueous antibody formulations described herein, the formulation is stable (e.g., % HMW by SEC ≤5%) at 40° C. for about 1 hour to about 8 weeks (e.g., about 1 hour to about 6 weeks, about 1 hour to about 4 weeks, about 1 hour to about 2 weeks, about 1 hour to about 1 week, about 1 hour to about 6 days, about 1 hour to about 4 days, about 1 hour to about 2 days, about 1 hour to about 1 day, about 1 hour to about 22 hours, about 1 hour to about 20 hours, about 1 hour to about 18 hours, about 1 hour to about 16 hours, about 1 hour to about 14 hours, about 1 hour to about 12 hours, about 1 hour to about 10 hours, about 1 hour to about 8 hours, about 1 hour to about 6 hours, about 1 hour to about 4 hours, about 1 hour to about 2 hours, about 2 hours to about 8 weeks, about 2 hours to about 6 weeks, about 2 hours to about 4 weeks, about 2 hours to about 2 weeks, about 2 hours to about 1 week, about 2 hours to about 6 days, about 2 hours to about 4 days, about 2 hours to about 2 days, about 2 hours to about 1 day, about 2 hours to about 22 hours, about 2 hours to about 20 hours, about 2 hours to about 18 hours, about 2 hours to about 16 hours, about 2 hours to about 14 hours, about 2 hours to about 12 hours, about 2 hours to about 10 hours, about 2 hours to about 8 hours, about 2 hours to about 6 hours, about 2 hours to about 4 hours, about 4 hours to about 8 weeks, about 4 hours to about 6 weeks, about 4 hours to about 4 weeks, about 4 hours to about 2 weeks, about 4 hours to about 1 week, about 4 hours to about 6 days, about 4 hours to about 4 days, about 4 hours to about 2 days, about 4 hours to about 1 day, about 4 hours to about 22 hours, about 4 hours to about 20 hours, about 4 hours to about 18 hours, about 4 hours to about 16 hours, about 4 hours to about 14 hours, about 4 hours to about 12 hours, about 4 hours to about 10 hours, about 4 hours to about 8 hours, about 4 hours to about 6 hours, about 6 hours to about 8 weeks, about 6 hours to about 6 weeks, about 6 hours to about 4 weeks, about 6 hours to about 2 weeks, about 6 hours to about 1 week, about 6 hours to about 6 days, about 6 hours to about 4 days, about 6 hours to about 2 days, about 6 hours to about 1 day, about 6 hours to about 22 hours, about 6 hours to about 20 hours, about 6 hours to about 18 hours, about 6 hours to about 16 hours, about 6 hours to about 14 hours, about 6 hours to about 12 hours, about 6 hours to about 10 hours, about 6 hours to about 8 hours, about 8 hours to about 8 weeks, about 8 hours to about 6 weeks, about 8 hours to about 4 weeks, about 8 hours to about 2 weeks, about 8 hours to about 1 week, about 8 hours to about 6 days, about 8 hours to about 4 days, about 8 hours to about 2 days, about 8 hours to about 1 day, about 8 hours to about 22 hours, about 8 hours to about 20 hours, about 8 hours to about 18 hours, about 8 hours to about 16 hours, about 8 hours to about 14 hours, about 8 hours to about 12 hours, about 8 hours to about 10 hours, about 10 hours to about 8 weeks, about 10 hours to about 6 weeks, about 10 hours to about 4 weeks, about 10 hours to about 2 weeks, about 10 hours to about 1 week, about 10 hours to about 6 days, about 10 hours to about 4 days, about 10 hours to about 2 days, about 10 hours to about 1 day, about 10 hours to about 22 hours, about 10 hours to about 20 hours, about 10 hours to about 18 hours, about 10 hours to about 16 hours, about 10 hours to about 14 hours, about 10 hours to about 12 hours, about 12 hours to about 8 weeks, about 12 hours to about 6 weeks, about 12 hours to about 4 weeks, about 12 hours to about 2 weeks, about 12 hours to about 1 week, about 12 hours to about 6 days, about 12 hours to about 4 days, about 12 hours to about 2 days, about 12 hours to about 1 day, about 12 hours to about 22 hours, about 12 hours to about 20 hours, about 12 hours to about 18 hours, about 12 hours to about 16 hours, about 12 hours to about 14 hours, about 14 hours to about 8 weeks, about 14 hours to about 6 weeks, about 14 hours to about 4 weeks, about 14 hours to about 2 weeks, about 14 hours to about 1 week, about 14 hours to about 6 days, about 14 hours to about 4 days, about 14 hours to about 2 days, about 14 hours to about 1 day, about 14 hours to about 22 hours, about 14 hours to about 20 hours, about 14 hours to about 18 hours, about 14 hours to about 16 hours, about 16 hours to about 8 weeks, about 16 hours to about 6 weeks, about 16 hours to about 4 weeks, about 16 hours to about 2 weeks, about 16 hours to about 1 week, about 16 hours to about 6 days, about 16 hours to about 4 days, about 16 hours to about 2 days, about 16 hours to about 1 day, about 16 hours to about 22 hours, about 16 hours to about 20 hours, about 16 hours to about 18 hours, about 18 hours to about 8 weeks, about 18 hours to about 6 weeks, about 18 hours to about 4 weeks, about 18 hours to about 2 weeks, about 18 hours to about 1 week, about 18 hours to about 6 days, about 18 hours to about 4 days, about 18 hours to about 2 days, about 18 hours to about 1 day, about 18 hours to about 22 hours, about 18 hours to about 20 hours, about 20 hours to about 8 weeks, about 20 hours to about 6 weeks, about 20 hours to about 4 weeks, about 20 hours to about 2 weeks, about 20 hours to about 1 week, about 20 hours to about 6 days, about 20 hours to about 4 days, about 20 hours to about 2 days, about 20 hours to about 1 day, about 20 hours to about 22 hours, about 22 hours to about 8 weeks, about 22 hours to about 6 weeks, about 22 hours to about 4 weeks, about 22 hours to about 2 weeks, about 22 hours to about 1 week, about 22 hours to about 6 days, about 22 hours to about 4 days, about 22 hours to about 2 days, about 22 hours to about 24 hours, about 1 day to about 8 weeks, about 1 day to about 6 weeks, about 1 day to about 4 weeks, about 1 day to about 2 weeks, about 1 day to about 1 week, about 1 day to about 6 days, about 1 day to about 4 days, about 1 day to about 2 days, about 2 days to about 8 weeks, about 2 days to about 6 weeks, about 2 days to about 4 weeks, about 2 days to about 2 weeks, about 2 days to about 1 week, about 2 days to about 6 days, about 2 days to about 4 days, about 4 days to about 8 weeks, about 4 days to about 6 weeks, about 4 days to about 4 weeks, about 4 days to about 2 weeks, about 4 days to about 1 week, about 4 days to about 6 days, about 1 week to about 8 weeks, about 1 week to about 6 weeks, about 1 week to about 4 weeks, about 1 week to about 2 weeks, about 2 weeks to about 8 weeks, about 2 weeks to about 6 weeks, about 2 weeks to about 4 weeks, about 4 weeks to about 8 weeks, about 4 weeks to about 6 weeks, or about 6 weeks to about 8 weeks (e.g., as determined using high-performance size exclusion chromatography).

In some embodiments, the formulation, e.g., before and/or after the addition of a salt (e.g., any of the salts described herein), has a viscosity of about 1 cP to about 50 cP, about 1 cP to about 45 cP, about 1 cP to about 40 cP, about 1 cP to about 35 cP, about 1 cP to about 30 cP, about 1 cP to about 25 cP, about 1 cP to about 20 cP, about 1 cP to about 15 cP, about 1 cP to about 10 cP, about 1 cP to about 5 cP, about 5 cP to about 50 cP, about 5 cP to about 45 cP, about 5 cP to about 40 cP, about 5 cP to about 35 cP, about 5 cP to about 30 cP, about 5 cP to about 25 cP, about 5 cP to about 20 cP, about 5 cP to about 15 cP, about 5 cP to about 10 cP, about 10 cP to about 50 cP, about 10 cP to about 45 cP, about 10 cP to about 40 cP, about 10 cP to about 35 cP, about 10 cP to about 30 cP, about 10 cP to about 25 cP, about 10 cP to about 20 cP, about 10 cP to about 15 cP, about 15 cP to about 50 cP, about 15 cP to about 45 cP, about 15 cP to about 40 cP, about 15 cP to about 35 cP, about 15 cP to about 30 cP, about 15 cP to about 25 cP, about 15 cP to about 20 cP, about 20 cP to about 50 cP, about 20 cP to about 45 cP, about 20 cP to about 40 cP, about 20 cP to about 35 cP, about 20 cP to about 30 cP, about 20 cP to about 25 cP, about 25 cP to about 50 cP, about 25 cP to about 45 cP, about 25 cP to about 40 cP, about 25 cP to about 35 cP, about 25 cP to about 30 cP, about 30 cP to about 50 cP, about 30 cP to about 45 cP, about 30 cP to about 40 cP, about 30 cP to about 35 cP, about 35 cP to about 50 cP, about 35 cP to about 45 cP, about 35 cP to about 40 cP, about 40 cP to about 50 cP, about 40 cP to about 45 cP, or about 45 cP to about 50 cP (e.g., as measured using a viscometer).

In some embodiments, the formulation can be suitable for subcutaneous administration, intravenous administration, intraarterial administration, intraocular administration, intraperitoneal administration, intramuscular administration, intraarticular administration, or interlaminar administration.

### Stabilizers

Some embodiments of any of the formulations described herein can further include a stabilizer (e.g., one or more stabilizers). Non-limiting examples of stabilizers include fructose, maltose, galactose, glucose, 0-mannose, sorbose, lactose, sucrose, trehalose, cellobiose, raffinose, melezitose, a maltodextrin, a dextran, starch, mannitol, xylitol, maltitol, lactitol, glucitol, sucrose, trehalose, raffinose, maltose, sorbitol, mannitol, an amino sugar, sodium chloride, and glycerol, and combinations thereof. Additional examples of stabilizers are known in the art. The final concentration of a stabilizer (or the final total concentration of one or more stabilizers) in any of the formulations described herein can be about 0.01 mM to about 1500 mM (or any of the subranges of this range described herein).

### Amino Acids

Some embodiments of any of the formulations described herein can further include an amino acid (e.g., one or more amino acids). Non-limiting examples of amino acids include arginine, lysine, histidine, proline, ornithine, isoleucine, leucine, alanine, glycine, glutamic acid, and aspartic acid, and combinations thereof. Additional examples of amino acids are known in the art. The final concentration of an amino acid (or a final total concentration of one or more amino acids) in any of the formulations described herein can be about 0.01 mM to about 750 mM (or any of the subranges of this range described herein).

### Surfactants

Some embodiments of any of the formulations described herein can further include a surfactant (e.g., one or more surfactants). Non-limiting examples of surfactants include sorbitan monocaprylate, sorbitan monolaurate, sorbitan monopalmitate, sorbitan trioleate, glycerine monocaprylate, glycerine monomyristate, glycerine monostearate, decaglyceryl monostearate, decaglyceryl distearate, decaglyceryl monolinoleate, polyoxyethylene sorbitan monolaurate, polyoxythylene sorbitan monocleate, polyoxyethylene sorbitan monostearate, polyoxyethylene sorbitan monopalmitate, polyoxyethyene sorbitan trioleate, polyoxyethylene sorbitan tristearate, polyoxyethylene sorbitol tetrastearate, polyoxyethylene sorbitol tetraoleate, polyoxyethylene glyceryl monostearate, polyethylene glycol distearate, polyoxyethylene lauryl ether, polyoxyethylene polyoxypropylene glycol, polyoxyethylene polyoxypropylene propyl ether, polyoxyethylene polyoxypropylene cetyl ether, polyoxyethylene nonylphenyl ether, polyoxythylene castor oil, polyoxyethylene hydrogenated castor oil, polyoxyethylene sorbitol beeswax, polyoxyethylene lanolin, polyoxyethylene stearic acid amide, sodium cetyl sulfate, sodium lauryl sulfate, sodium oleyl sulfate, sodium polyoxyethylene lauryl sulfate, sodium lauryl sulfosuccinate ester, lecithin, a glycerophosopholipid, a sphingophospholipid, polysorbate 20, polysorbate 40, polysorbate 60, polysorbate 80, poloxamer 188, triton-X, sodium lauryl sulfate, polyethylene glycol, and propylene glycol. Additional examples of surfactants are known in the art.

The final concentration of a surfactant (or a final total concentration of one or more surfactants) in any of the formulations described herein can be about 0.001% w/v to about 2% w/v, about 0.001% w/v to about 1.8% w/v, about 0.001% w/v to about 1.6% w/v, about 0.001% w/v to about 1.4% w/v, about 0.001% w/v to about 1.2% w/v, about 0.001% w/v to about 1.0% w/v, about 0.001% w/v to about 0.9% w/v, about 0.001% w/v to about 0.8% w/v, about 0.001% w/v to about 0.7% w/v, about 0.001% w/v to about 0.6% w/v, about 0.001% w/v to about 0.5% w/v, about 0.001% w/v to about 0.45% w/v, about 0.001% w/v to about 0.40% w/v, about 0.001% w/v to about 0.35% w/v, about 0.001% w/v to about 0.30% w/v, about 0.001% w/v to about 0.25% w/v, about 0.001% w/v to about 0.20% w/v, about 0.001% w/v to about 0.15% w/v, about 0.001% w/v to about 0.10% w/v, about 0.001% w/v to about 0.05% w/v, about 0.001% w/v to about 0.01% w/v, about 0.001% w/v to about 0.005% w/v, about 0.005% w/v to about 2.0% w/v, about 0.005% w/v to about 1.8% w/v, about 0.005% w/v to about 1.6% w/v, about 0.005% w/v to about 1.4% w/v, about 0.005% w/v to about 1.2% w/v, about 0.005% w/v to about 1.0% w/v, about 0.005% w/v to about 0.9% w/v, about 0.005% w/v to about 0.8% w/v, about 0.005% w/v to about 0.7% w/v, about 0.005% w/v to about 0.6% w/v, about 0.005% w/v to about 0.5% w/v, about 0.005% w/v to about 0.45% w/v, about 0.005% w/v to about 0.40% w/v, about 0.005% w/v to about 0.35% w/v, about 0.005% w/v to about 0.30% w/v, about 0.005% w/v to about 0.25% w/v, about 0.005% w/v to about 0.20% w/v, about 0.005% w/v to about 0.15% w/v, about 0.005% w/v to about 0.10% w/v, about 0.005% w/v to about 0.05% w/v, about 0.005% w/v to about 0.01% w/v, about 0.01% w/v to about 2.0% w/v, about 0.01% w/v to about 1.8% w/v, about 0.01% w/v to about 1.6% w/v, about 0.01% w/v to about 1.4% w/v, about 0.01% w/v to about 1.2% w/v, about 0.01% w/v to about 1.0% w/v, about 0.01% w/v to about 0.9% w/v, about 0.01% w/v to about 0.8% w/v, about 0.01% w/v to about 0.7% w/v, about 0.01% w/v to about 0.6% w/v, about 0.01% w/v to about 0.5% w/v, about 0.01% w/v to about 0.45% w/v, about 0.01% w/v to about 0.40% w/v, about 0.01% w/v to about 0.35% w/v, about 0.01% w/v to about 0.30% w/v, about 0.01% w/v to about 0.25% w/v, about 0.01% w/v to about 0.20% w/v, about 0.01% w/v to about 0.15% w/v, about 0.01% w/v to about 0.10% w/v, about 0.01% w/v to about 0.05% w/v, about 0.05% w/v to about 2.0% w/v, about 0.05% w/v to about 1.8% w/v, about 0.05% w/v to about 1.6% w/v, about 0.05% w/v to about 1.4% w/v, about 0.05% w/v to about 1.2% w/v, about 0.05% w/v to about 1.0% w/v, about 0.05% w/v to about 0.9% w/v, about 0.05% w/v to about 0.8% w/v, about 0.05% w/v to about 0.7% w/v, about 0.05% w/v to about 0.6% w/v, about 0.05% w/v to about 0.5% w/v, about 0.05% w/v to about 0.45% w/v, about 0.05% w/v to about 0.40% w/v, about 0.05% w/v to about 0.35% w/v, about 0.05% w/v to about 0.30% w/v, about 0.05% w/v to about 0.25% w/v, about 0.05% w/v to about 0.20% w/v, about 0.05% w/v to about 0.15% w/v, about 0.05% w/v to about 0.10% w/v, about 0.10% w/v to about 2.0% w/v, about 0.10% w/v to about 1.8% w/v, about 0.10% w/v to about 1.6% w/v, about 0.10% w/v to about 1.4% w/v, about 0.10% w/v to about 1.2% w/v, about 0.10% w/v to about 1.0% w/v, about 0.10% w/v to about 0.9% w/v, about 0.10% w/v to about 0.8% w/v, about 0.10% w/v to about 0.7% w/v, about 0.10% w/v to about 0.6% w/v, about 0.10% w/v to about 0.5% w/v, about 0.10% w/v to about 0.45% w/v, about 0.10% w/v to about 0.40% w/v, about 0.10% w/v to about 0.35% w/v, about 0.10% w/v to about 0.30% w/v, about 0.10% w/v to about 0.25% w/v, about 0.10% w/v to about 0.20% w/v, about 0.10% w/v to about 0.15% w/v, about 0.15% w/v to about 2.0% w/v, about 0.15% w/v to about 1.8% w/v, about 0.15% w/v to about 1.6% w/v, about 0.15% w/v to about 1.4% w/v, about 0.15% w/v to about 1.2% w/v, about 0.15% w/v to about 1.0% w/v, about 0.15% w/v to about 0.9% w/v, about 0.15% w/v to about 0.8% w/v, about 0.15% w/v to about 0.7% w/v, about 0.15% w/v to about 0.6% w/v, about 0.15% w/v to about 0.5% w/v, about 0.15% w/v to about 0.45% w/v, about 0.15% w/v to about 0.40% w/v, about 0.15% w/v to about 0.35% w/v, about 0.15% w/v to about 0.30% w/v, about 0.15% w/v to about 0.25% w/v, about 0.15% w/v to about 0.20% w/v, about 0.20% w/v to about 2.0% w/v, about 0.20% w/v to about 1.8% w/v, about 0.20% w/v to about 1.6% w/v, about 0.20% w/v to about 1.4% w/v, about 0.20% w/v to about 1.2% w/v, about 0.20% w/v to about 1.0% w/v, about 0.20% w/v to about 0.9% w/v, about 0.20% w/v to about 0.8% w/v, about 0.20% w/v to about 0.7% w/v, about 0.20% w/v to about 0.6% w/v, about 0.20% w/v to about 0.5% w/v, about 0.20% w/v to about 0.45% w/v, about 0.20% w/v to about 0.40% w/v, about 0.20% w/v to about 0.35% w/v, about 0.20% w/v to about 0.30% w/v, about 0.20% w/v to about 0.25% w/v, about 0.25% w/v to about 2.0% w/v, about 0.25% w/v to about 1.8% w/v, about 0.25% w/v to about 1.6% w/v, about 0.25% w/v to about 1.4% w/v, about 0.25% w/v to about 1.2% w/v, about 0.25% w/v to about 1.0% w/v, about 0.25% w/v to about 0.9% w/v, about 0.25% w/v to about 0.8% w/v, about 0.25% w/v to about 0.7% w/v, about 0.25% w/v to about 0.6% w/v, about 0.25% w/v to about 0.5% w/v, about 0.25% w/v to about 0.45% w/v, about 0.25% w/v to about 0.40% w/v, about 0.25% w/v to about 0.35% w/v, about 0.25% w/v to about 0.30% w/v, about 0.30% w/v to about 2.0% w/v, about 0.30% w/v to about 1.8% w/v, about 0.30% w/v to about 1.6% w/v, about 0.30% w/v to about 1.4% w/v, about 0.30% w/v to about 1.2% w/v, about 0.30% w/v to about 1.0% w/v, about 0.30% w/v to about 0.9% w/v, about 0.30% w/v to about 0.8% w/v, about 0.30% w/v to about 0.7% w/v, about 0.30% w/v to about 0.6% w/v, about 0.30% w/v to about 0.5% w/v, about 0.30% w/v to about 0.45% w/v, about 0.30% w/v to about 0.40% w/v, about 0.30% w/v to about 0.35% w/v, about 0.35% w/v to about 2.0% w/v, about 0.35% w/v to about 1.8% w/v, about 0.35% w/v to about 1.6% w/v, about 0.35% w/v to about 1.4% w/v, about 0.35% w/v to about 1.2% w/v, about 0.35% w/v to about 1.0% w/v, about 0.35% w/v to about 0.9% w/v, about 0.35% w/v to about 0.8% w/v, about 0.35% w/v to about 0.7% w/v, about 0.35% w/v to about 0.6% w/v, about 0.35% w/v to about 0.5% w/v, about 0.35% w/v to about 0.45% w/v, about 0.35% w/v to about 0.40% w/v, about 0.40% w/v to about 2.0% w/v, about 0.40% w/v to about 1.8% w/v, about 0.40% w/v to about 1.6% w/v, about 0.40% w/v to about 1.4% w/v, about 0.40% w/v to about 1.2% w/v, about 0.40% w/v to about 1.0% w/v, about 0.40% w/v to about 0.9% w/v, about 0.40% w/v to about 0.8% w/v, about 0.40% w/v to about 0.7% w/v, about 0.40% w/v to about 0.6% w/v, about 0.40% w/v to about 0.5% w/v, about 0.40% w/v to about 0.45% w/v, about 0.45% w/v to about 2.0% w/v, about 0.45% w/v to about 1.8% w/v, about 0.45% w/v to about 1.6% w/v, about 0.45% w/v to about 1.4% w/v, about 0.45% w/v to about 1.2% w/v, about 0.45% w/v to about 1.0% w/v, about 0.45% w/v to about 0.9% w/v, about 0.45% w/v to about 0.8% w/v, about 0.45% w/v to about 0.7% w/v, about 0.45% w/v to about 0.6% w/v, about 0.45% w/v to about 0.5% w/v, about 0.5% w/v to about 2.0% w/v, about 0.5% w/v to about 1.8% w/v, about 0.5% w/v to about 1.6% w/v, about 0.5% w/v to about 1.4% w/v, about 0.5% w/v to about 1.2% w/v, about 0.5% w/v to about 1.0% w/v, about 0.5% w/v to about 0.9% w/v, about 0.5% w/v to about 0.8% w/v, about 0.5% w/v to about 0.7% w/v, about 0.5% w/v to about 0.6% w/v, about 0.6% w/v to about 2.0% w/v, about 0.6% w/v to about 1.8% w/v, about 0.6% w/v to about 1.6% w/v, about 0.6% w/v to about 1.4% w/v, about 0.6% w/v to about 1.2% w/v, about 0.6% w/v to about 1.0% w/v, about 0.6% w/v to about 0.9% w/v, about 0.6% w/v to about 0.8% w/v, about 0.6% w/v to about 0.7% w/v, about 0.7% w/v to about 2.0% w/v, about 0.7% w/v to about 1.8% w/v, about 0.7% w/v to about 1.6% w/v, about 0.7% w/v to about 1.4% w/v, about 0.7% w/v to about 1.2% w/v, about 0.7% w/v to about 1.0% w/v, about 0.7% w/v to about 0.9% w/v, about 0.7% w/v to about 0.8% w/v, about 0.8% w/v to about 2.0% w/v, about 0.8% w/v to about 1.8% w/v, about 0.8% w/v to about 1.6% w/v, about 0.8% w/v to about 1.4% w/v, about 0.8% w/v to about 1.2% w/v, about 0.8% w/v to about 1.0% w/v, about 0.8% w/v to about 0.9% w/v, about 0.9% w/v to about 2.0% w/v, about 0.9% w/v to about 1.8% w/v, about 0.9% w/v to about 1.6% w/v, about 0.9% w/v to about 1.4% w/v, about 0.9% w/v to about 1.2% w/v, about 0.9% w/v to about 1.0% w/v, about 1.0% w/v to about 2.0% w/v, about 1.0% w/v to about 1.8% w/v, about 1.0% w/v to about 1.6% w/v, about 1.0% w/v to about 1.4% w/v, about 1.0% w/v to about 1.2% w/v, about 1.2% w/v to about 2.0% w/v, about 1.2% w/v to about 1.8% w/v, about 1.2% w/v to about 1.6% w/v, about 1.2% w/v to about 1.4% w/v, about 1.4% w/v to about 2.0% w/v, about 1.4% w/v to about 1.8% w/v, about 1.4% w/v to about 1.6% w/v, about 1.6% w/v to about 2.0% w/v, about 1.6% w/v to about 1.8% w/v, or about 1.8% w/v to about 2.0% w/v.

The final concentration of a surfactant (or a final total concentration of one or more surfactants) in any of the formulations described herein can be about 0.001 mg/mL to about 50 mg/mL, about 0.001 mg/mL to about 45 mg/mL, about 0.001 mg/mL to about 40 mg/mL, about 0.001 mg/mL to about 35 mg/mL, about 0.001 mg/mL to about 30 mg/mL, about 0.001 mg/mL to about 28 mg/mL, about 0.001 mg/mL to about 26 mg/mL, about 0.001 mg/mL to about 24 mg/mL, about 0.001 mg/mL to about 22 mg/mL, about 0.001 mg/mL to about 20 mg/mL, about 0.001 mg/mL to about 18 mg/mL, about 0.001 mg/mL to about 16 mg/mL, about 0.001 mg/mL to about 14 mg/mL, about 0.001 mg/mL to about 12 mg/mL, about 0.001 mg/mL to about 10 mg/mL, about 0.001 mg/mL to about 8 mg/mL, about 0.001 mg/mL to about 6 mg/mL, about 0.001 mg/mL to about 5 mg/mL, about 0.001 mg/mL to about 4 mg/mL, about 0.001 mg/mL to about 3 mg/mL, about 0.001 mg/mL to about 2 mg/mL, about 0.001 mg/mL to about 1 mg/mL, about 0.001 mg/mL to about 0.5 mg/mL, about 0.001 mg/mL to about 0.1 mg/mL, about 0.001 mg/mL to about 0.05 mg/mL, about 0.001 mg/mL to about 0.005 mg/mL, about 0.005 mg/mL to about 50 mg/mL, about 0.005 mg/mL to about 45 mg/mL, about 0.005 mg/mL to about 40 mg/mL, about 0.005 mg/mL to about 35 mg/mL, about 0.005 mg/mL to about 30 mg/mL, about 0.005 mg/mL to about 28 mg/mL, about 0.005 mg/mL to about 26 mg/mL, about 0.005 mg/mL to about 24 mg/mL, about 0.005 mg/mL to about 22 mg/mL, about 0.005 mg/mL to about 20 mg/mL, about 0.005 mg/mL to about 18 mg/mL, about 0.005 mg/mL to about 16 mg/mL, about 0.005 mg/mL to about 14 mg/mL, about 0.005 mg/mL to about 12 mg/mL, about 0.005 mg/mL to about 10 mg/mL, about 0.005 mg/mL to about 8 mg/mL, about 0.005 mg/mL to about 6 mg/mL, about 0.005 mg/mL to about 5 mg/mL, about 0.005 mg/mL to about 4 mg/mL, about 0.005 mg/mL to about 3 mg/mL, about 0.005 mg/mL to about 2 mg/mL, about 0.005 mg/mL to about 1 mg/mL, about 0.005 mg/mL to about 0.5 mg/mL, about 0.005 mg/mL to about 0.1 mg/mL, about 0.005 mg/mL to about 0.05 mg/mL, about 0.05 mg/mL to about 50 mg/mL, about 0.05 mg/mL to about 45 mg/mL, about 0.05 mg/mL to about 40 mg/mL, about 0.05 mg/mL to about 35 mg/mL, about 0.05 mg/mL to about 30 mg/mL, about 0.05 mg/mL to about 28 mg/mL, about 0.05 mg/mL to about 26 mg/mL, about 0.05 mg/mL to about 24 mg/mL, about 0.05 mg/mL to about 22 mg/mL, about 0.05 mg/mL to about 20 mg/mL, about 0.05 mg/mL to about 18 mg/mL, about 0.05 mg/mL to about 16 mg/mL, about 0.05 mg/mL to about 14 mg/mL, about 0.05 mg/mL to about 12 mg/mL, about 0.05 mg/mL to about 10 mg/mL, about 0.05 mg/mL to about 8 mg/mL, about 0.05 mg/mL to about 6 mg/mL, about 0.05 mg/mL to about 5 mg/mL, about 0.05 mg/mL to about 4 mg/mL, about 0.05 mg/mL to about 3 mg/mL, about 0.05 mg/mL to about 2 mg/mL, about 0.05 mg/mL to about 1 mg/mL, about 0.05 mg/mL to about 0.5 mg/mL, about 0.05 mg/mL to about 0.1 mg/mL, about 0.1 mg/mL to about 50 mg/mL, about 0.1 mg/mL to about 45 mg/mL, about 0.1 mg/mL to about 40 mg/mL, about 0.1 mg/mL to about 35 mg/mL, about 0.1 mg/mL to about 30 mg/mL, about 0.1 mg/mL to about 28 mg/mL, about 0.1 mg/mL to about 26 mg/mL, about 0.1 mg/mL to about 24 mg/mL, about 0.1 mg/mL to about 22 mg/mL, about 0.1 mg/mL to about 20 mg/mL, about 0.1 mg/mL to about 18 mg/mL, about 0.1 mg/mL to about 16 mg/mL, about 0.1 mg/mL to about 14 mg/mL, about 0.1 mg/mL to about 12 mg/mL, about 0.1 mg/mL to about 10 mg/mL, about 0.1 mg/mL to about 8 mg/mL, about 0.1 mg/mL to about 6 mg/mL, about 0.1 mg/mL to about 5 mg/mL, about 0.1 mg/mL to about 4 mg/mL, about 0.1 mg/mL to about 3 mg/mL, about 0.1 mg/mL to about 2 mg/mL, about 0.1 mg/mL to about 1 mg/mL, about 0.1 mg/mL to about 0.5 mg/mL, about 0.5 mg/mL to about 50 mg/mL, about 0.5 mg/mL to about 45 mg/mL, about 0.5 mg/mL to about 40 mg/mL, about 0.5 mg/mL to about 35 mg/mL, about 0.5 mg/mL to about 30 mg/mL, about 0.5 mg/mL to about 28 mg/mL, about 0.5 mg/mL to about 26 mg/mL, about 0.5 mg/mL to about 24 mg/mL, about 0.5 mg/mL to about 22 mg/mL, about 0.5 mg/mL to about 20 mg/mL, about 0.5 mg/mL to about 18 mg/mL, about 0.5 mg/mL to about 16 mg/mL, about 0.5 mg/mL to about 14 mg/mL, about 0.5 mg/mL to about 12 mg/mL, about 0.5 mg/mL to about 10 mg/mL, about 0.5 mg/mL to about 8 mg/mL, about 0.5 mg/mL to about 6 mg/mL, about 0.5 mg/mL to about 5 mg/mL, about 0.5 mg/mL to about 4 mg/mL, about 0.5 mg/mL to about 3 mg/mL, about 0.5 mg/mL to about 2 mg/mL, about 0.5 mg/mL to about 1 mg/mL, about 1 mg/mL to about 50 mg/mL, about 1 mg/mL to about 45 mg/mL, about 1 mg/mL to about 40 mg/mL, about 1 mg/mL to about 35 mg/mL, about 1 mg/mL to about 30 mg/mL, about 1 mg/mL to about 28 mg/mL, about 1 mg/mL to about 26 mg/mL, about 1 mg/mL to about 24 mg/mL, about 1 mg/mL to about 22 mg/mL, about 1 mg/mL to about 20 mg/mL, about 1 mg/mL to about 18 mg/mL, about 1 mg/mL to about 16 mg/mL, about 1 mg/mL to about 14 mg/mL, about 1 mg/mL to about 12 mg/mL, about 1 mg/mL to about 10 mg/mL, about 1 mg/mL to about 8 mg/mL, about 1 mg/mL to about 6 mg/mL, about 1 mg/mL to about 5 mg/mL, about 1 mg/mL to about 4 mg/mL, about 1 mg/mL to about 3 mg/mL, about 1 mg/mL to about 2 mg/mL, about 2 mg/mL to about 50 mg/mL, about 2 mg/mL to about 45 mg/mL, about 2 mg/mL to about 40 mg/mL, about 2 mg/mL to about 35 mg/mL, about 2 mg/mL to about 30 mg/mL, about 2 mg/mL to about 28 mg/mL, about 2 mg/mL to about 26 mg/mL, about 2 mg/mL to about 24 mg/mL, about 2 mg/mL to about 22 mg/mL, about 2 mg/mL to about 20 mg/mL, about 2 mg/mL to about 18 mg/mL, about 2 mg/mL to about 16 mg/mL, about 2 mg/mL to about 14 mg/mL, about 2 mg/mL to about 12 mg/mL, about 2 mg/mL to about 10 mg/mL, about 2 mg/mL to about 8 mg/mL, about 2 mg/mL to about 6 mg/mL, about 2 mg/mL to about 5 mg/mL, about 2 mg/mL to about 4 mg/mL, about 2 mg/mL to about 3 mg/mL, about 3 mg/mL to about 50 mg/mL, about 3 mg/mL to about 45 mg/mL, about 3 mg/mL to about 40 mg/mL, about 3 mg/mL to about 35 mg/mL, about 3 mg/mL to about 30 mg/mL, about 3 mg/mL to about 28 mg/mL, about 3 mg/mL to about 26 mg/mL, about 3 mg/mL to about 24 mg/mL, about 3 mg/mL to about 22 mg/mL, about 3 mg/mL to about 20 mg/mL, about 3 mg/mL to about 18 mg/mL, about 3 mg/mL to about 16 mg/mL, about 3 mg/mL to about 14 mg/mL, about 3 mg/mL to about 12 mg/mL, about 3 mg/mL to about 10 mg/mL, about 3 mg/mL to about 8 mg/mL, about 3 mg/mL to about 6 mg/mL, about 3 mg/mL to about 5 mg/mL, about 3 mg/mL to about 4 mg/mL, about 4 mg/mL to about 50 mg/mL, about 4 mg/mL to about 45 mg/mL, about 4 mg/mL to about 40 mg/mL, about 4 mg/mL to about 35 mg/mL, about 4 mg/mL to about 30 mg/mL, about 4 mg/mL to about 28 mg/mL, about 4 mg/mL to about 26 mg/mL, about 4 mg/mL to about 24 mg/mL, about 4 mg/mL to about 22 mg/mL, about 4 mg/mL to about 20 mg/mL, about 4 mg/mL to about 18 mg/mL, about 4 mg/mL to about 16 mg/mL, about 4 mg/mL to about 14 mg/mL, about 4 mg/mL to about 12 mg/mL, about 4 mg/mL to about 10 mg/mL, about 4 mg/mL to about 8 mg/mL, about 4 mg/mL to about 6 mg/mL, about 4 mg/mL to about 5 mg/mL, about 5 mg/mL to about 50 mg/mL, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 28 mg/mL, about 5 mg/mL to about 26 mg/mL, about 5 mg/mL to about 24 mg/mL, about 5 mg/mL to about 22 mg/mL, about 5 mg/mL to about 20 mg/mL, about 5 mg/mL to about 18 mg/mL, about 5 mg/mL to about 16 mg/mL, about 5 mg/mL to about 14 mg/mL, about 5 mg/mL to about 12 mg/mL, about 5 mg/mL to about 10 mg/mL, about 5 mg/mL to about 8 mg/mL, about 5 mg/mL to about 6 mg/mL, about 6 mg/mL to about 50 mg/mL, about 6 mg/mL to about 45 mg/mL, about 6 mg/mL to about 40 mg/mL, about 6 mg/mL to about 35 mg/mL, about 6 mg/mL to about 30 mg/mL, about 6 mg/mL to about 28 mg/mL, about 6 mg/mL to about 26 mg/mL, about 6 mg/mL to about 24 mg/mL, about 6 mg/mL to about 22 mg/mL, about 6 mg/mL to about 20 mg/mL, about 6 mg/mL to about 18 mg/mL, about 6 mg/mL to about 16 mg/mL, about 6 mg/mL to about 14 mg/mL, about 6 mg/mL to about 12 mg/mL, about 6 mg/mL to about 10 mg/mL, about 6 mg/mL to about 8 mg/mL, about 8 mg/mL to about 50 mg/mL, about 8 mg/mL to about 45 mg/mL, about 8 mg/mL to about 40 mg/mL, about 8 mg/mL to about 35 mg/mL, about 8 mg/mL to about 30 mg/mL, about 8 mg/mL to about 28 mg/mL, about 8 mg/mL to about 26 mg/mL, about 8 mg/mL to about 24 mg/mL, about 8 mg/mL to about 22 mg/mL, about 8 mg/mL to about 20 mg/mL, about 8 mg/mL to about 18 mg/mL, about 8 mg/mL to about 16 mg/mL, about 8 mg/mL to about 14 mg/mL, about 8 mg/mL to about 12 mg/mL, about 8 mg/mL to about 10 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 28 mg/mL, about 10 mg/mL to about 26 mg/mL, about 10 mg/mL to about 24 mg/mL, about 10 mg/mL to about 22 mg/mL, about 10 mg/mL to about 20 mg/mL, about 10 mg/mL to about 18 mg/mL, about 10 mg/mL to about 16 mg/mL, about 10 mg/mL to about 14 mg/mL, about 10 mg/mL to about 12 mg/mL, about 12 mg/mL to about 50 mg/mL, about 12 mg/mL to about 45 mg/mL, about 12 mg/mL to about 40 mg/mL, about 12 mg/mL to about 35 mg/mL, about 12 mg/mL to about 30 mg/mL, about 12 mg/mL to about 28 mg/mL, about 12 mg/mL to about 26 mg/mL, about 12 mg/mL to about 24 mg/mL, about 12 mg/mL to about 22 mg/mL, about 12 mg/mL to about 20 mg/mL, about 12 mg/mL to about 18 mg/mL, about 12 mg/mL to about 16 mg/mL, about 12 mg/mL to about 14 mg/mL, about 14 mg/mL to about 50 mg/mL, about 14 mg/mL to about 45 mg/mL, about 14 mg/mL to about 40 mg/mL, about 14 mg/mL to about 35 mg/mL, about 14 mg/mL to about 30 mg/mL, about 14 mg/mL to about 28 mg/mL, about 14 mg/mL to about 26 mg/mL, about 14 mg/mL to about 24 mg/mL, about 14 mg/mL to about 22 mg/mL, about 14 mg/mL to about 20 mg/mL, about 14 mg/mL to about 18 mg/mL, about 14 mg/mL to about 16 mg/mL, about 16 mg/mL to about 50 mg/mL, about 16 mg/mL to about 45 mg/mL, about 16 mg/mL to about 40 mg/mL, about 16 mg/mL to about 35 mg/mL, about 16 mg/mL to about 30 mg/mL, about 16 mg/mL to about 28 mg/mL, about 16 mg/mL to about 26 mg/mL, about 16 mg/mL to about 24 mg/mL, about 16 mg/mL to about 22 mg/mL, about 16 mg/mL to about 20 mg/mL, about 16 mg/mL to about 18 mg/mL, about 18 mg/mL to about 50 mg/mL, about 18 mg/mL to about 45 mg/mL, about 18 mg/mL to about 40 mg/mL, about 18 mg/mL to about 35 mg/mL, about 18 mg/mL to about 30 mg/mL, about 18 mg/mL to about 28 mg/mL, about 18 mg/mL to about 26 mg/mL, about 18 mg/mL to about 24 mg/mL, about 18 mg/mL to about 22 mg/mL, about 18 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 28 mg/mL, about 20 mg/mL to about 26 mg/mL, about 20 mg/mL to about 24 mg/mL, about 20 mg/mL to about 22 mg/mL, about 22 mg/mL to about 50 mg/mL, about 22 mg/mL to about 45 mg/mL, about 22 mg/mL to about 40 mg/mL, about 22 mg/mL to about 35 mg/mL, about 22 mg/mL to about 30 mg/mL, about 22 mg/mL to about 28 mg/mL, about 22 mg/mL to about 26 mg/mL, about 22 mg/mL to about 24 mg/mL, about 24 mg/mL to about 50 mg/mL, about 24 mg/mL to about 45 mg/mL, about 24 mg/mL to about 40 mg/mL, about 24 mg/mL to about 35 mg/mL, about 24 mg/mL to about 30 mg/mL, about 24 mg/mL to about 28 mg/mL, about 24 mg/mL to about 26 mg/mL, about 26 mg/mL to about 50 mg/mL, about 26 mg/mL to about 45 mg/mL, about 26 mg/mL to about 40 mg/mL, about 26 mg/mL to about 35 mg/mL, about 26 mg/mL to about 30 mg/mL, about 26 mg/mL to about 28 mg/mL, about 28 mg/mL to about 50 mg/mL, about 28 mg/mL to about 45 mg/mL, about 28 mg/mL to about 40 mg/mL, about 28 mg/mL to about 35 mg/mL, about 28 mg/mL to about 30 mg/mL, about 30 mg/mL to about 50 mg/mL, about 30 mg/mL to about 45 mg/mL, about 30 mg/mL to about 40 mg/mL, about 30 mg/mL to about 35 mg/mL, about 35 mg/mL to about 50 mg/mL, about 35 mg/mL to about 45 mg/mL, about 35 mg/mL to about 40 mg/mL, about 40 mg/mL to about 50 mg/mL, about 40 mg/mL to about 45 mg/mL, or about 45 mg/mL to about 50 mg/mL.

### Injection Devices

Also provided herein are injection devices that include any of the aqueous antibody formulations described herein (e.g., one or more doses of any of the aqueous antibody formations described herein). For example, the injection device can be a pre-loaded syringe. Non-limiting examples of such pre-loaded syringes are known in the art.

### Kits

Also provided herein are kits that include any of the injection devices provided herein. Also provided herein are kits that include one or more vials containing any of the aqueous antibody formulations described herein. In some embodiments, any of the kits provided herein can further include instructions for administration of any of the aqueous antibody formulations to a subject in need thereof.

### Methods of Making a Formulation

Also provided herein are methods of making any of the aqueous antibody formulations described herein that include mixing or combining: (i) an antibody or an antigen-binding fragment thereof (e.g., any of the exemplary antibodies or antigen-binding antibody fragments described herein); (ii) a buffer (e.g., any of the buffers or one or more of any of the buffers described herein); (iii) a salt (or one or more salts) selected from the group of magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate; (iv) a stabilizer; (v) a surfactant; and (vi) water (e.g., sterile water), where (i) to (vi) are mixed or combined in amounts sufficient to generate any of the aqueous antibody formulations described herein.

Some embodiments of these methods can further include filtering mixed or combined (i) to (vi). Some embodiments of these methods can further include disposing or placing the formulation into a sterile vile (e.g., a vacuum-sealed, sterile vial) or a syringe (e.g., a sterile syringe).

Some embodiments of any of these methods can further include adding or mixing with (i) to (vi), one or more (e.g., two or three) of a stabilizer (e.g., one or more of any of the exemplary stabilizers described herein or known in the art), an amino acid (e.g., one or more of any of the exemplary amino acids described herein or known in the art), and a surfactant (e.g., one or more of any of the exemplary surfactants described herein or known in the art), e.g., in amounts sufficient to result in any of the aqueous antibody formulations described herein.

Some embodiments of any of these methods can further include adding or mixing together with the other components one or more additional therapeutic agent(s).

As can be appreciated by those in the art, the order that each component of the formulation is added can be varied. For example, the antibody or antigen-binding antibody fragment can be a lyophilized solid that is dissolved in a buffered aqueous solution including the salt and the buffer. In another example, a solid comprising the antibody or antigen-binding antibody fragment (in the form of a lyophilized powder) and the salt, can be dissolved in a buffered aqueous solution (comprising the buffer). In some embodiments, a solid comprising the antibody or antigen-binding fragment (in the form of a lyphophilized powder), the buffer, and the salt, is dissolved in water (e.g., sterile water).

Also provided herein are methods of making an aqueous antibody formulations that include mixing or combining: (i) an antibody or an antigen-binding fragment thereof; and (ii) a salt selected from the group of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate; (iii) a stabilizer); (iv) a surfactant); and (v) sterile water, wherein (i) to (v) are mixed or combined in amounts sufficient to generate any of the aqueous antibody formulations described herein. In some embodiments of the methods described herein, the method does not include mixing or combining a buffer with (i) to (v) and the methods results in a buffer-free aqueous antibody formulation.

Some embodiments of these methods can further include filtering mixed or combined (i) to (v). Some embodiments of these methods can further include disposing or placing the formulation into a sterile vile (e.g., a vacuum-sealed, sterile vial) or a syringe (e.g., a sterile syringe).

Some embodiments of any of these methods can further include adding or mixing with (i) to (v), one or more (e.g., two or three) of a stabilizer (e.g., one or more of any of the exemplary stabilizers described herein or known in the art), an amino acid (e.g., any of the exemplary amino acids described herein except for histidine and arginine), and a surfactant (e.g., one or more of any of the exemplary surfactants described herein or known in the art), e.g., in amounts sufficient to result in any of the aqueous antibody formulations described herein.

Some embodiments of any of these methods can further include adding or mixing together with the other components one or more additional therapeutic agent(s). The mixing or combining can be formed by pipetting, vortexing, rocking agitation, or hand agitation. Other means for performing the mixing or combining are known in the art.

### Methods of Treating a Subject

Also provided herein are methods of treating a subject in need thereof that include administering to the subject (e.g., any of the subjects described herein) a therapeutically effective amount of any of the aqueous antibody formulations provided herein. In some embodiments of any of these methods, the aqueous antibody formulation can be administered by intravenous, intramuscular, intraperitoneal, subcutaneous, intraarterial, intraocular, intraocular, intraarticular, or interlaminar administration. In some embodiments of any of these methods, the subject can be administered one or more doses of the aqueous antibody formulation.

In some embodiments, the subject has been identified or diagnosed (e.g., previously identified or diagnosed as having a disease or condition that will benefit from treatment with the antibody or the antigen-binding antibody fragment that is present in the aqueous antibody formulation).

In some embodiments of any of the methods described herein, the subject can also be administered one or more additional therapeutic agents. In some embodiments, the one or more additional therapeutic agents can be administered to the subject at the substantially the same time as the aqueous antibody formulation. In some embodiments, the one or more additional therapeutic agents can be administered to the subject to the subject before or after the administration of the aqueous antibody formulation to the subject.

### EXAMPLES

The invention is further described in the following examples, which do not limit the scope of the invention described in the claims.

### Exemplary Components of Antibody Formulations

Two Fc engineered mAbs (anti-CXCR3-DE an IgG1 (“antibody A”) and anti-CD38-ADE an IgG1 (“antibody B”)) and one traditional mAb (anti-CD38 an IgG1 (“antibody C”)) were employed for conformational and kinetic stability studies, as well as viscosity measurements. Various formulations were prepared by combination and proper pH adjustment of the following excipients: L-Arginine, L-Arginine Hydrochloride, L-Lysine, L-Lysine Hydrochloride, L-Histidine Hydrochloride, L-Aspartic Acid, L-Glutamic Acid, L-Glycine, L-Valine, L-Methionine, L-Serine, Hydrochloric Acid, Sulfuric Acid, Acetic Acid, Sodium Hydroxide, Lithium Hydroxide, Lithium Bromide, Magnesium Aspartate, Magnesium Glutamate, Magnesium Sulfate, Magnesium Chloride, Sodium Thiocyanate, Sodium Acetate, Ammonium Sulfate, Sucrose, Trehalose, Sorbitol, and Glycerol.

### Exemplary Sample Preparation

All sugar, polyol, amino acid, amino acid salt and metal salt solutions were prepared in 10 mM histidine buffer between pH 5.5 and pH 6.2. For thermal (conformational) stability measurements, 1 mg/mL mAb formulations were prepared by spiking excipient solutions with stock mAb solution at a ratio of ˜ 1/50 volume to volume. For kinetic stability studies and viscosity measurements, stock mAb solutions were concentrated and buffer exchanged into the various excipient solutions using Vivaspin-20 30 kilodalton molecular weight cut off (MWCO) membranes (General Electric). MAb formulations were concentrated up to >180 mg/mL and subsequently diluted down to 150 mg/mL with the corresponding excipient solution.

### Exemplary Aqueous Antibody Formulations

In some embodiments, the aqueous antibody formulation comprises about 0.1 to about 400 mg/mL of an antibody or antigen-binding fragment thereof (e.g., any of the antibodies or antigen-binding fragments described herein), about 0 to about 100 mM of a buffer (e.g., any of the buffers described herein), about 1 to about 750 mM of a stabilizing salt or a viscosity salt (e.g., any of the stabilizing salts or viscosity salts described herein), about 0.001 to about 0.2% of a surfactant (e.g., any of the surfactants described herein), about 0% to about 10% of a sugar or a polyol, wherein the aqueous antibody formulation has a pH of about 4 to about 8. See, e.g., Embodiment A in Table 1.

In some embodiments, the aqueous antibody formulation comprises about 0.1 to about 300 mg/mL of an antibody or antigen-binding fragment thereof (e.g., any of the antibodies or antigen-binding fragments described herein), about 0 to about 50 mM of a buffer (e.g., any of the buffers described herein), about 5 to about 500 mM of a stabilizing salt or a viscosity salt (e.g., any of the stabilizing salts or viscosity salts described herein), about 0.01 to about 0.1% of a surfactant (e.g., any of the surfactants described herein), about 1% to about 8% of a sugar or a polyol, wherein the aqueous antibody formulation has a pH of about 4.5 to about 7.5. See, e.g., Embodiment B in Table 1.

In some embodiments, the aqueous antibody formulation comprises about 0.1 to about 200 mg/mL of an antibody or antigen-binding fragment thereof (e.g., any of the antibodies or antigen-binding fragments described herein), about 0 to about 25 mM of a buffer (e.g., any of the buffers described herein), about 25 to about 300 mM of a stabilizing salt or a viscosity salt (e.g., any of the stabilizing salts or viscosity salts described herein), about 0.01 to about 0.06% of a surfactant (e.g., any of the surfactants described herein), about 1.5% to about 8% of a sugar or a polyol, wherein the aqueous antibody formulation has a pH of about 5 to about 7. See, e.g., Embodiment C in Table 1.

In some embodiments, the aqueous antibody formulation comprises about 0 to about 10 mM of a buffer (e.g., any of the buffers described herein), about 300 to about 750 mM of a stabilizing salt or a viscosity salt (e.g., any of the stabilizing salts or viscosity salts described herein), wherein the aqueous antibody formulation has a pH of about 5 to about 6.5. See, e.g., Embodiment D in Table 1.

TABLE 1
Exemplary Embodiments of Antibody Formulations

| Component | Embodiment A | | Embodiment B | | Embodiment C | | Embodiment D | |
|---|---|---|---|---|---|---|---|---|
| Active (IgG) | 0.1-400 | mg/mL | 0.1-300 | mg/mL | 0.1-250 | mg/mL | 0.1-200 | mg/mL |
| Buffer | 0-100 | mM | 0-50 | mM | 0-25 | mM | 0-10 | mM |
| pH | 4-8 | | 4.5-7.5 | | 5-7 | | 5-6.5 | |
| Stabilizing/ | 1-750 | mM | 5-500 | mM | 25-300 | mM | 300-750 | mM |
| viscosity | | | | | | | | |
| modifying salt | | | | | | | | |

| | | | | |
|---|---|---|---|---|
| Sugars, Polyols | 0-10% | 1-8% | 1.5-8% | 2.0-6% |
| Surfactant | 0.001-0.2% | 0.01-0.1% | 0.01-0.06% | 0.02-0.06% |

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium acetate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium acetate, about 2% of sucrose, about 0.05% of polysorbate 80, wherein the aqueous antibody formulation has a pH of about 5.5. See, e.g., Embodiment 1 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium aspartate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium aspartate, about 2% of sucrose, about 0.05% of polysorbate 80, wherein the aqueous antibody formulation has a pH of about 5.5. See, e.g., Embodiment 2 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium glutamate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium glutamate, about 2% of sucrose, about 0.05% of polysorbate 80, wherein the aqueous antibody formulation has a pH of about 5.5. See, e.g., Embodiment 3 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium sulfate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium sulfate, about 2% of sucrose, about 0.05% of polysorbate 80, wherein the aqueous antibody formulation has a pH of about 5.5. See, e.g., Embodiment 4 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium aspartate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 5 mM of a histidine buffer, about 200 mM of magnesium aspartate, about 2% of sucrose, about 0.05% of polysorbate 80, wherein the aqueous antibody formulation has a pH of about 5.5. See, e.g., Embodiment 5 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium glutamate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 5 mM of a histidine buffer, about 200 mM of magnesium glutamate, about 2% of sucrose, about 0.05% of polysorbate 80, wherein the aqueous antibody formulation has a pH of about 5.5. See, e.g., Embodiment 6 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of an acetate buffer, about 100 to about 300 mM of magnesium aspartate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of an acetate buffer, about 200 mM of magnesium aspartate, about 2% of sucrose, about 0.05% of polysorbate 80, wherein the aqueous antibody formulation has a pH of about 5.0. See, e.g., Embodiment 7 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of an acetate buffer, about 100 to about 300 mM of magnesium glutamate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of an acetate buffer, about 200 mM of magnesium glutamate, about 2% of sucrose, about 0.05% of polysorbate 80, wherein the aqueous antibody formulation has a pH of about 5.0. See, e.g., Embodiment 8 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium aspartate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium aspartate, about 2% of sucrose, about 0.05% of polysorbate 80, wherein the aqueous antibody formulation has a pH of about 6.0. See, e.g., Embodiment 9 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium glutamate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium glutamate, about 2% of sucrose, about 0.05% of polysorbate 80, wherein the aqueous antibody formulation has a pH of about 6.0. See, e.g., Embodiment 10 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of lithium aspartate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of lithium aspartate, about 2% of sucrose, about 0.05% of polysorbate 80, wherein the aqueous antibody formulation has a pH of about 5.5. See, e.g., Embodiment 11 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of lithium glutamate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of lithium glutamate, about 2% of sucrose, about 0.05% of polysorbate 80, wherein the aqueous antibody formulation has a pH of about 5.5. See, e.g., Embodiment 12 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of sodium aspartate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of sodium aspartate, about 2% of sucrose, about 0.05% of polysorbate 80, wherein the aqueous antibody formulation has a pH of about 5.5. See, e.g., Embodiment 13 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of sodium glutamate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of sodium glutamate, about 2% of sucrose, about 0.05% of polysorbate 80, wherein the aqueous antibody formulation has a pH of about 5.5. See, e.g., Embodiment 14 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 200 to about 750 mM of arginine aspartate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 300 mM of arginine aspartate, about 2% of sucrose, about 0.05% of polysorbate 80, wherein the aqueous antibody formulation has a pH of about 5.5. See, e.g., Embodiment 15 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 200 to about 750 mM of arginine glutamate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 300 mM of arginine glutamate, about 2% of sucrose, about 0.05% of polysorbate 80, wherein the aqueous antibody formulation has a pH of about 5.5. See, e.g., Embodiment 16 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 200 to about 750 mM of lysine aspartate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 300 mM of lysine aspartate, about 2% of sucrose, about 0.05% of polysorbate 80, wherein the aqueous antibody formulation has a pH of about 5.5. See, e.g., Embodiment 17 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 200 to about 750 mM of lysine glutamate, about 1% to about 8% of sucrose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 300 mM of lysine glutamate, about 2% of sucrose, about 0.05% of polysorbate 80, wherein the aqueous antibody formulation has a pH of about 5.5. See, e.g., Embodiment 18 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium aspartate, about 1% to about 8% of trehalose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium aspartate, about 2% of trehalose, about 0.05% of polysorbate 80, wherein the aqueous antibody formulation has a pH of about 5.5. See, e.g., Embodiment 19 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium glutamate, about 1% to about 8% of trehalose, about 0.01% to about 0.1% of polysorbate 80, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium glutamate, about 2% of trehalose, about 0.05% of polysorbate 80, wherein the aqueous antibody formulation has a pH of about 5.5. See, e.g., Embodiment 20 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium aspartate, about 1% to about 8% of sucrose, about 0.01% to about 0.1% of polysorbate 20, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium aspartate, about 2% of sucrose, about 0.05% of polysorbate 20, wherein the aqueous antibody formulation has a pH of about 5.5. See, e.g., Embodiment 21 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium glutamate, about 1% to about 8% of sucrose, about 0.01% to about 0.1% of polysorbate 20, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium glutamate, about 2% of sucrose, about 0.05% of polysorbate 20, wherein the aqueous antibody formulation has a pH of about 5.5. See, e.g., Embodiment 22 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium aspartate, about 1% to about 8% of sucrose, about 0.01% to about 0.1% of poloxamer 188, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium aspartate, about 2% of sucrose, about 0.05% of poloxamer 188, wherein the aqueous antibody formulation has a pH of about 5.5. See, e.g., Embodiment 23 in Table 2.

In some embodiments, the aqueous antibody formulation comprises about 0.1 to about 200 mg/mL of an antibody or antigen-binding fragment thereof (e.g., any of the antibodies or antigen-binding fragments described herein), about 1 to about 100 mM of a histidine buffer, about 100 to about 300 mM of magnesium glutamate, about 1% to about 8% of sucrose, about 0.01% to about 0.1% of poloxamer 188, wherein the aqueous antibody formulation has a pH of about 4.5 to about 7.5. In some embodiments, the aqueous antibody formulation comprises about 0.1 to about 200 mg/mL of an antibody or antigen-binding fragment thereof (e.g., any of the antibodies or antigen-binding fragments described herein), about 10 mM of a histidine buffer, about 200 mM of magnesium glutamate, about 2% of sucrose, about 0.05% of poloxamer 188, wherein the aqueous antibody formulation has a pH of about 5.5. See, e.g., Embodiment 24 in Table 2.

TABLE 2
Exemplary Embodiments of Antibody Formulations

| | Components | | | | | |
|---|---|---|---|---|---|---|
| Formulation # | Active (IgG) | Buffer | Salt | Stabilizer | Surfactant | pH |
| 1 | 0.1-200 | 10 mM | 200 mM | 2% | 0.05% | 5.5 |
| | mg/mL | Histidine | Mg. Ace | sucrose | polysorbate 80 | |
| 2 | 0.1-200 | 10 mM | 200 mM | 2% | 0.05% | 5.5 |
| | mg/mL | Histidine | Mg. Asp | sucrose | polysorbate 80 | |
| 3 | 0.1-200 | 10 mM | 200 mM | 2% | 0.05% | 5.5 |
| | mg/mL | Histidine | Mg.Glu | sucrose | polysorbate 80 | |
| 4 | 0.1-200 | 10 mM | 200 mM | 2% | 0.05% | 5.5 |
| | mg/mL | Histidine | Mg.Sul | sucrose | polysorbate 80 | |
| 5 | 0.1-200 | 5 mM | 200 mM | 2% | 0.05% | 5.5 |
| | mg/mL | Histidine | Mg.Asp | sucrose | polysorbate 80 | |
| 6 | 0.1-200 | 5 mM | 200 mM | 2% | 0.05% | 5.5 |
| | mg/mL | Histidine | Mg.Glu | sucrose | polysorbate 80 | |
| 7 | 0.1-200 | 10 mM | 200 mM | 2% | 0.05% | 5.0 |
| | mg/mL | Acetate | Mg.Asp | sucrose | polysorbate 80 | |
| 8 | 0.1-200 | 10 mM | 200 mM | 2% | 0.05% | 5.0 |
| | mg/mL | Acetate | Mg.Glu | sucrose | polysorbate 80 | |
| 9 | 0.1-200 | 10 mM | 200 mM | 2% | 0.05% | 6.0 |
| | mg/mL | Histidine | Mg.Asp | sucrose | polysorbate 80 | |
| 10 | 0.1-200 | 10 mM | 200 mM | 2% | 0.05% | 6.0 |
| | mg/mL | Histidine | Mg.Glu | sucrose | polysorbate 80 | |
| 11 | 0.1-200 | 10 mM | 200 mM | 2% | 0.05% | 5.5 |
| | mg/mL | Histidine | Li.Asp | sucrose | polysorbate 80 | |
| 12 | 0.1-200 | 10 mM | 200 mM | 2% | 0.05% | 5.5 |
| | mg/mL | Histidine | Li.Glu | sucrose | polysorbate 80 | |
| 13 | 0.1-200 | 10 mM | 200 mM | 2% | 0.05% | 5.5 |
| | mg/mL | Histidine | Na.Asp | sucrose | polysorbate 80 | |
| 14 | 0.1-200 | 10 mM | 200 mM | 2% | 0.05% | 5.5 |
| | mg/mL | Histidine | Na.Glu | sucrose | polysorbate 80 | |
| 15 | 0.1-200 | 10 mM | >300 mM | 2% | 0.05% | 5.5 |
| | mg/mL | Histidine | Arg.Asp | sucrose | polysorbate 80 | |
| 16 | 0.1-200 | 10 mM | >300 mM | 2% | 0.05% | 5.5 |
| | mg/mL | Histidine | Arg.Glu | sucrose | polysorbate 80 | |
| 17 | 0.1-200 | 10 mM | >300 mM | 2% | 0.05% | 5.5 |
| | mg/mL | Histidine | Lys.Asp | sucrose | polysorbate 80 | |
| 18 | 0.1-200 | 10 mM | >300 mM | 2% | 0.05% | 5.5 |
| | mg/mL | Histidine | Lys.Glu | sucrose | polysorbate 80 | |
| 19 | 0.1-200 | 10 mM | 200 mM | 2% | 0.05% | 5.5 |
| | mg/mL | Histidine | Mg.Asp | trehalose | polysorbate 80 | |
| 20 | 0.1-200 | 10 mM | 200 mM | 2% | 0.05% | 5.5 |
| | mg/mL | Histidine | Mg.Glu | trehalose | polysorbate 80 | |
| 21 | 0.1-200 | 10 mM | 200 mM | 2% | 0.05% | 5.5 |
| | mg/mL | Histidine | Mg.Asp | sucrose | polysorbate 20 | |
| 22 | 0.1-200 | 10 mM | 200 mM | 2% | 0.05% | 5.5 |
| | mg/mL | Histidine | Mg.Glu | sucrose | polysorbate 20 | |
| 23 | 0.1-200 | 10 mM | 200 mM | 2% | 0.5% | 5.5 |
| | mg/mL | Histidine | Mg.Asp | sucrose | poloxamer 188 | |
| 24 | 0.1-200 | 10 mM | 200 mM | 2% | 0.5% | 5.5 |
| | mg/mL | Histidine | Mg.Glu | sucrose | poloxamer 188 | |

Mg.Glu: magnesium glutamate; Mg.Ace: magnesium acetate; Mg.Asp: magnesium aspartate; Mg.Sul: magnesium sulfate; Li.Asp: lithium aspartate; Li.Glu: lithium glutamate; Na.Asp: sodium aspartate; Na.Glu: sodium glutamate; Arg.Asp: arginine aspartate; Arg.Glu: arginine glutamate; Lys.Asp: lysine aspartate; Lys.Glu: lysine glutamate.

Thermal (Conformational) Stability by Differential Scanning calorimetry (DSC)

Thermal (conformational) stability was assessed by measuring the lowest unfolding temperature, Tₘ₁, of mAbs on a capillary DSC system (Malvern). The impact of both the identity and concentration of different excipients on increasing the Tₘ₁ value was used to rank order their effectiveness at improving conformational stability. Measurements were conducted by ramping the temperature of the solution from 15° C. to 110° C. at a rate of 1° C./min. The temperature value at the maximum of the first peak was designated as Tₘ₁.

### Kinetic Stability by Size Exclusion Chromatography (SEC)

Kinetic stability was assessed by periodically measuring the amount of aggregates generated in mAb formulations stored at 40° C. for 4 weeks. SEC was used to quantify the levels of both aggregated and non-aggregated mAb molecules after 0, 1, 2, and 4 weeks of storage. Samples were measured by eluting 50 μg of total mAb off of a TSKgel UP-SW3000 column (Tosoh Co.) at a rate 0.43 mL/min with 40 mM Phosphate and 150 mM Sodium Chloride at pH 7.2 (detection wavelength: 280 nm). Aggregates were designated as high molecular weight species (HMWS) that eluted faster than non-aggregated mAb monomers; HMWS % was calculated by dividing the amount of aggregate generated by the sum of total aggregated+non-aggregated mAb molecules. The initial aggregation rate, k_{agg}, was taken as the slope of a plot of HMWS % vs. storage time.

### Viscosity Measurements of mAb Formulations

The viscosities of 150 mg/mL mAb formulations were measured on an initium rheometer (Rheosense). MAb formulations were forced through a microchannel at a single shear rate and pressure was measured at 20° C.

### Example 1: Stabilization of an Fc-Mutant mAb

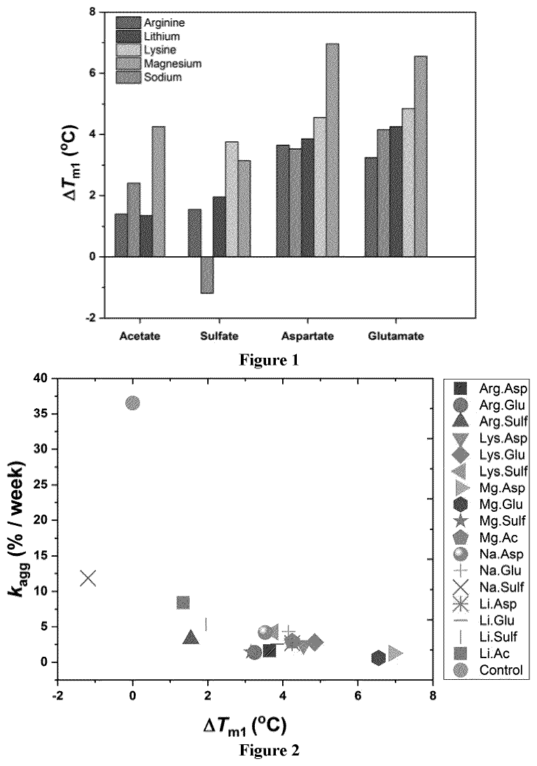

An Fc engineered mAb (antibody A) with a relatively low CH2 domain unfolding temperature (Tₘ₁), compared to traditional mAbs, was selected. Solutions of the mAb were prepared at 1 mg/mL in 10 or 20 mM histidine buffer at pH 5.5. The excipient solutions were prepared at 20 mM and 200 mM. Thermodynamic/conformational stability was evaluated by differential scanning calorimetry (DSC). Further, solutions of the mAb were prepared at 25 or 150 mg/mL concentration in the same buffer-excipient-pH solutions and kinetic or storage stability, as measured by rates of aggregation (k_{agg}), was evaluated at 5° C., 25° C. and 40° C. Excipients were then assessed based on their effectiveness in increasing Tₘ₁ (ΔTₘ₁) and/or decreasing k_{agg} for antibody A in solution. Based on the identity of the salt used, Tₘ₁ could be increased by as much as 7° C. with magnesium salts generally resulting in the most effect (FIG. 1). Arginine salts generally resulted in the least but still significant increase for antibody A. Of the anions, aspartates and glutamates were generally most effective in increasing the Tₘ₁. For k_{agg} (40° C., 150 mg/mL antibody A), a general decrease was observed with increasing ΔTₘ₁ (FIG. 2). Based on the identity of the salt, a roughly 35× decrease in the aggregation rate was noted compared to the control formulation. Magnesium salts generally resulted in the most decrease in k_{agg} followed by arginine, lysine, lithium and sodium.

### Example 2: Stabilization of Other Fc-Mutant mAb

Based on the results of Example 1, a subset of excipients was selected and their effectiveness as stabilizers was evaluated using another Fc-engineered mAb, with a relatively low Tₘ₁ (antibody B).

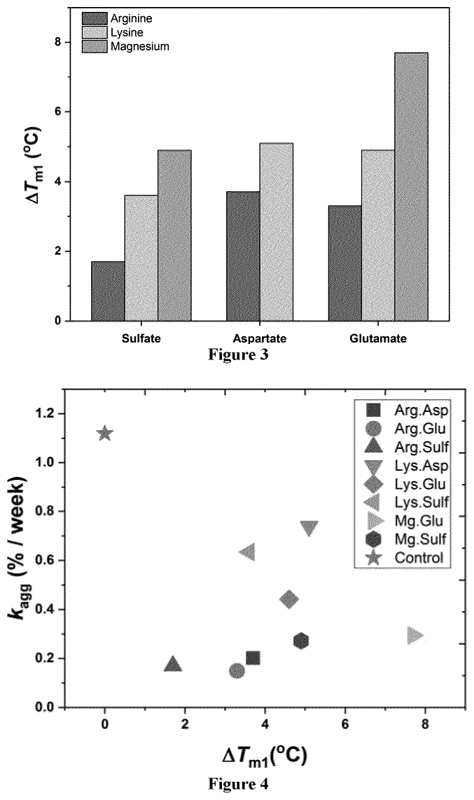

Solutions of antibody B (1 mg/mL) in 10 mM histidine buffer at pH 5.5 and 6.2 were prepared for conformational stability measurements. Solutions of antibody B (50 mg/mL) were prepared in the same buffer-excipient at pH 6.2 for kinetic stability (40° C.) analysis. Similar to the effect observed for antibody A, an increase in Tₘ₁ was observed for antibody B in a salt-dependent manner (FIG. 3). Magnesium salts generally exhibited the most increase. For anions, glutamates were observed to be most effective in increasing Tₘ₁ followed by aspartates and sulfates. Consistent with the effect on Tₘ₁, a decrease in k_{agg} was also noted in the presence of salts (FIG. 4). Magnesium salts generally were most effective in increasing k_{agg}.

### Example 3: Stabilization of an Fc-Mutant mAb (Antibody A) as a Function of Excipient Concentration

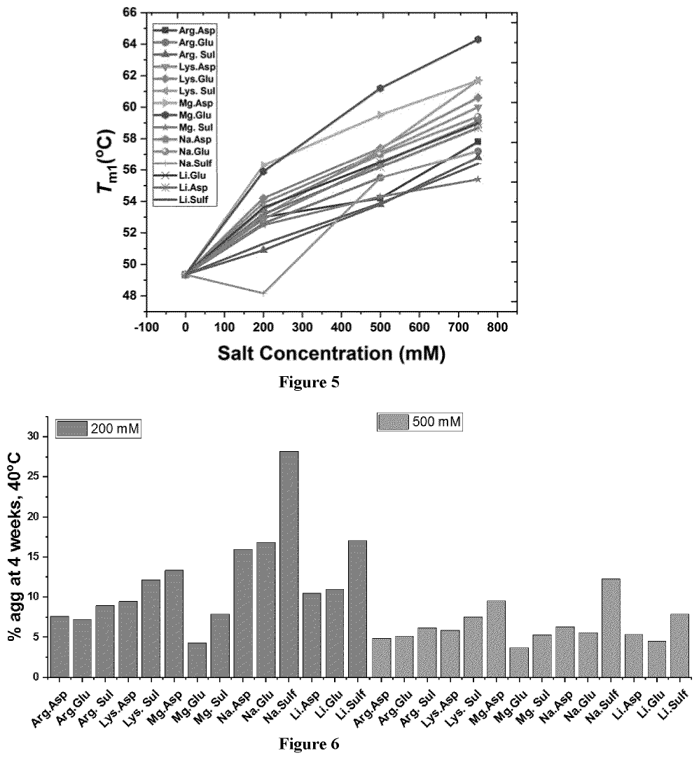

Excipients were evaluated at higher concentrations, 500 mM and 750 mM, for their stabilizing effect on antibody A in solution. All excipients were shown to improve the conformational stability of antibody A by increasing Tₘ₁ to in a concentration dependent manner (FIG. 5). Magnesium salts generally exhibited the most increase in Tₘ₁; ˜15° C. at 750 mM for magnesium glutamate. Furthermore, results from kinetic stability study (FIG. 6) showed that these same excipients at 500 mM further decreased aggregation and k_{agg} compared to 200 mM concentration used in Example 1.

### Example 4: Stabilization of a Wild-Type mAb

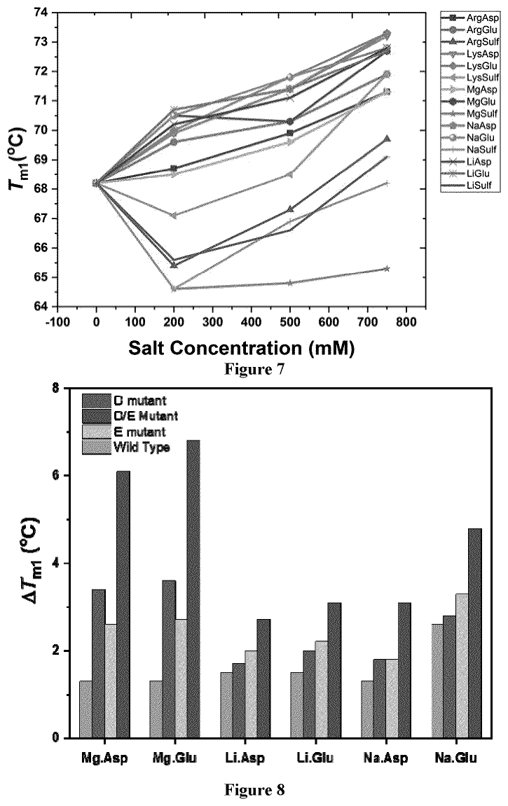

The same excipients as those used in Example 2 were also evaluated for their stabilizing effect on a traditional mAb (antibody C), which has a much higher Tₘ₁ than the Fc-mutants used in our studies. Most excipients moderately improved the conformational stability with increasing concentration (200 mM, 500 mM, and 750 mM) with the exception of sulfates (FIG. 7). Aspartate and glutamate salts were generally better in stabilizing wild-type antibodies.

### Example 5: Stabilization of Another Wild-Type mAb and its Single and Double Amino Acid Mutants

Aspartic and glutamic acid salts of magnesium, lithium and sodium at 200 mM concentration were evaluated for their stabilizing effect on wild-type anti-CEACAM5 antibody and its antibody D, antibody E and antibody DE mutants. All salts improved the conformational stability of the four antibodies studied with the effect being most prominent for the antibody DE mutant (FIG. 8). Of the salts studied, magnesium salts were most effective at increasing Tₘ₁.

### Example 6: Effects of Excipients on Viscosity and Osmolality of mAb Solutions

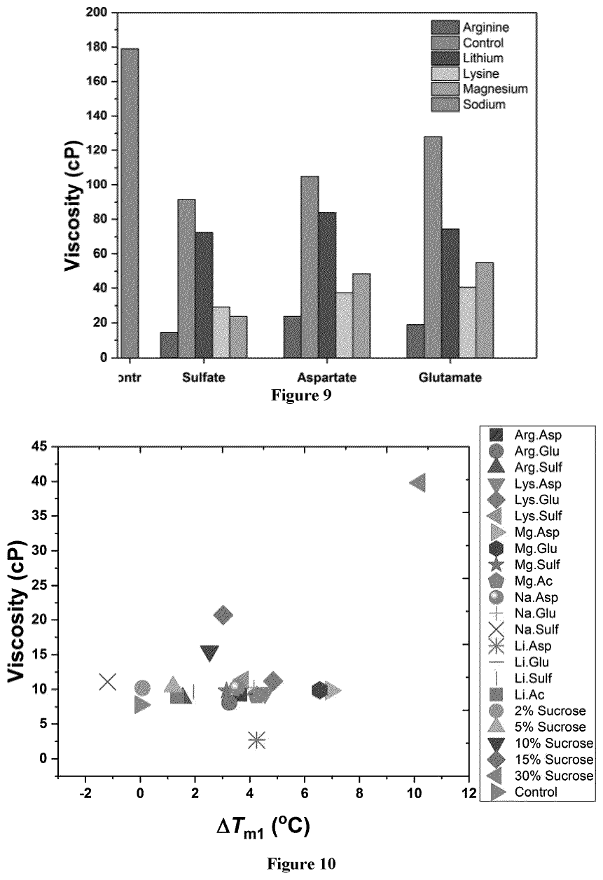

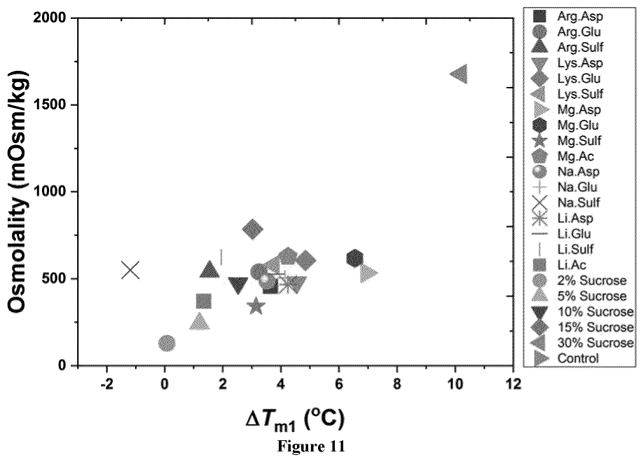

Effect of excipients at 200 mM concentration on viscosity of antibody solutions was also evaluated. Viscosity of antibody A and antibody C solutions (˜150 mg/mL) in presence and absence of aforementioned excipients was measured and compared with that in presence of traditional stabilizing excipients such as sucrose. Results indicated that the selected excipients either (i) significantly reduced solution viscosity compared to the control solutions (FIG. 9) and/or (ii) resulted in solutions of lower viscosity (FIG. 10) and lower osmolality (FIG. 11) in comparison to sucrose at equipotent concentrations. For antibody C, solution viscosity could be reduced by 10×, compared to control, with arginine, lysine and magnesium salts being most effective followed by sodium and lithium (FIG. 9). Antibody A solutions on the contrary were not too viscous to begin with (Control formulation in FIG. 10). Addition of sucrose to these solutions, for improving conformational stability resulted in a significant increase in solution viscosity especially at concentrations higher than 10%. Such an increase in solution viscosity was not observed with any of the salts studied. The effect of the two excipient classes on solution viscosity was most apparent for solutions exhibiting a ΔTₘ₁ of >3° C. Results indicate that the viscosity liability associated with the use of sucrose at concentrations that are not too far from those generally employed for stabilizing protein formulations (˜8%) is essentially absent for excipients listed in this invention. A near-identical effect was observed in the context of solution osmolality as well (FIG. 11).

Solutions containing 15% and 30% sucrose exhibited higher osmolality compared to 200 mM salt solutions without necessarily having a significantly higher stabilizing effect as measured by ΔTₘ₁.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### SEQUENCE INFORMATION

Amino acid sequences are provided, corresponding to heavy and light chains of the exemplary antibodies disclosed herein.

Heavy Chain, Antibody A - Anti-CXCR3-DE (S239D, I332E)- Fc Mutant
(SEQ ID NO: 1)
EVQLLESGGGLVQPGGSLRLSCAASGFTFTSYAMSWVRQAPGKGLEWVA
TISHGGTYTYYPDSVKGRFTISRDNAKNTLYLQMNSLRAEDTAVYYCARHPIYSG
NYQGYFDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNT
KVDKKVEPKSCDKTHTCPPCPAPELLGGPDVFLFPPKPKDTLMISRTPEVTCVVV
DVSHEDPEVKFNWYVDGVEVHNAKTKPREEQYNSTYRVVSVLTVLHQDWLNG
KEYKCKVSNKALPAPEEKTISKAKGQPREPQVYTLPPSRDELTKNQVSLTCLVKG
FYPSDIAVEWESNGQPENNYKTTPPVLDSDGSFFLYSKLTVDKSRWQQGNVFSCS
VMHEALHNHYTQKSLSLSPG
Light Chain, Antibody A - Anti-CXCR3-DE (S239D, I332E)- Fc Mutant
(SEQ ID NO: 2)
DIQLTQSPSFLSASVGDRVTITCRASSGVNYLYWYQQKPGKAPKLWIYFTS
TLASGVPSRFSGSGSGNEYTLTISSLQPEDFATYYCQQFTSSPYTFGQGTKLEIKRT
VAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Heavy Chain, Antibody C - Anti-CD38 (Isatuximab)- Wild Type IgG1 Fc
(SEQ ID NO: 3)
QVQLVQSGAEVAKPGTSVKLSCKASGYTFTDYWMQWVKQRPGQGLEWI
GTIYPGDGDTGYAQKFQGKATLTADKSSKTVYMHLSSLASEDSAVYYCARGDYY
GSNSLDYWGQGTSVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVM
HEALHNHYTQKSLSLSPG
Light Chain, Antibody C- Anti-CD38 (Isatuximab)- Wild Type IgG1 Fc
(SEQ ID NO: 4)
DIVMTQSHLSMSTSLGDPVSITCKASQDVSTVVAWYQQKPGQSPRRLIYS
ASYRYIGVPDRFTGSGAGTDFTFTISSVQAEDLAVYYCQQHYSPPYTFGGGTKLEI
KRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Heavy Chain, Antibody B - Anti-CD38-ADE (G236A, S239D, I332E)- Fc Mutant
(SEQ ID NO: 5)
QVQLVQSGAEVAKPGTSVKLSCKASGYTFTDYWMQWVKQRPGQGLEWI
GTIYPGDGDTGYAQKFQGKATLTADKSSKTVYMHLSSLASEDSAVYYCARGDYY
GSNSLDYWGQGTSVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTK
VDKKVEPKSCDKTHTCPPCPAPELLAGPDVFLFPPKPKDTLMISRTPEVTCVVVD
VSHEDPEVKFNWYVDGVEVHNAKTKPREEQYNSTYRVVSVLTVLHQDWLNGK
EYKCKVSNKALPAPEEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSV
MHEALHNHYTQKSLSLSPG
Light Chain, Antibody B - Anti-CD38-ADE (G236A, S239D, I332E)- Fc Mutant
(SEQ ID NO: 6)
DIVMTQSHLSMSTSLGDPVSITCKASQDVSTVVAWYQQKPGQSPRRLIYS
ASYRYIGVPDRFTGSGAGTDFTFTISSVQAEDLAVYYCQQHYSPPYTFGGGTKLEI
KRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Heavy Chain, Anti-CEACAM5 - Wild Type IgG1 Fc
(SEQ ID NO: 7)
EVQLQESGPGLVKPGGSLSLSCAASGFVFSSYDMSWVRQTPERGLEWVAY
ISSGGGITYAPSTVKGRFTVSRDNAKNTLYLQMNSLTSEDTAVYYCAAHYFGSSG
PFAYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEA
LHNHYTQKSLSLSPG
Light Chain, Anti-CEACAM5 - Wild Type IgG1 Fc
(SEQ ID NO: 8)
DIQMTQSPASLSASVGDRVTITCRASENIFSYLAWYQQKPGKSPKLLVYNT
RTLAEGVPSRFSGSGSGTDFSLTISSLQPEDFATYYCQHHYGTPFTFGSGTKLEIKR
TVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
Heavy Chain, Antibody D - Anti-CEACAM5 (S239D)- Fc Mutant
(SEQ ID NO: 9)
EVQLQESGPGLVKPGGSLSLSCAASGFVFSSYDMSWVRQTPERGLEWVAY
ISSGGGITYAPSTVKGRFTVSRDNAKNTLYLQMNSLTSEDTAVYYCAAHYFGSSG
PFAYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPDVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEA
LHNHYTQKSLSLSPG
Light Chain, Antibody D - Anti-CEACAM5 (S239D) - Fc Mutant
(SEQ ID NO: 10)
DIQMTQSPASLSASVGDRVTITCRASENIFSYLAWYQQKPGKSPKLLVYNT
RTLAEGVPSRFSGSGSGTDFSLTISSLQPEDFATYYCQHHYGTPFTFGSGTKLEIKR
TVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
Heavy Chain, Antibody E - Anti-CEACAM5 (I332E) - Fc Mutant
(SEQ ID NO: 11)
EVQLQESGPGLVKPGGSLSLSCAASGFVFSSYDMSWVRQTPERGLEWVAY
ISSGGGITYAPSTVKGRFTVSRDNAKNTLYLQMNSLTSEDTAVYYCAAHYFGSSG
PFAYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPEEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHE
ALHNHYTQKSLSLSPG
Light Chain, Antibody E- Anti-CEACAM5 (I332E) - Fc Mutant
(SEQ ID NO: 12)
DIQMTQSPASLSASVGDRVTITCRASENIFSYLAWYQQKPGKSPKLLVYNT
RTLAEGVPSRFSGSGSGTDFSLTISSLQPEDFATYYCQHHYGTPFTFGSGTKLEIKR
TVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
Heavy Chain, Antibody DE - Anti-CEACAM5 (S239D, I332E) - Fc Mutant
(SEQ ID NO: 13)
EVQLQESGPGLVKPGGSLSLSCAASGFVFSSYDMSWVRQTPERGLEWVAY
ISSGGGITYAPSTVKGRFTVSRDNAKNTLYLQMNSLTSEDTAVYYCAAHYFGSSG
PFAYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPDVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPEEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHE
ALHNHYTQKSLSLSPG
Light Chain, Antibody DE - Anti-CEACAM5 (S239D, I332E) - Fc Mutant
(SEQ ID NO: 14)
DIQMTQSPASLSASVGDRVTITCRASENIFSYLAWYQQKPGKSPKLLVYNT
RTLAEGVPSRFSGSGSGTDFSLTISSLQPEDFATYYCQHHYGTPFTFGSGTKLEIKR
TVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC

What is claimed is:

1. An aqueous antibody formulation comprising:
about 0.1 mg/mL to about 500 mg/mL of an antibody or an antigen-binding fragment thereof;
about 1 mM to about 100 mM of a buffer; and
about 1 mM to about 750 mM of a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate,
wherein the formulation has a pH of about 4 to about 8.

2. An aqueous antibody formulation comprising:
about 0.1 mg/mL to about 500 mg/mL of an antibody or an antigen-binding fragment thereof; and
about 1 mM to about 750 mM of a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate,
wherein the formulation has a pH of about 4 to about 8.

3. The formulation of claim 2, wherein the formulation is a buffer-free formulation.

4. The formulation of any one of claims 1-3, wherein the salt is magnesium glutamate, magnesium acetate, magnesium aspartate, or magnesium sulfate, or a combination thereof.

5. The formulation of claim 4, wherein the salt is magnesium glutamate.

6. The formulation of claim 4, wherein the salt is magnesium acetate.

7. The formulation of claim 4, wherein the salt is magnesium aspartate.

8. The formulation of claim 4, wherein the salt is magnesium sulfate.

9. The formulation of any one of claims 1-8, wherein the formulation comprises about 10 mM to about 750 mM of the salt.

10. The formulation of claim 9, wherein the formulation comprises about 20 mM to about 750 mM of the salt.

11. The formulation of any one of claims 1-3, wherein the salt is sodium acetate, sodium aspartate, sodium glutamate, or sodium sulfate.

12. The formulation of claim 11, wherein the salt is sodium acetate.

13. The formulation of claim 11, wherein the salt is sodium aspartate.

14. The formulation of claim 11, wherein the salt is sodium glutamate.

15. The formulation of claim 11, wherein the salt is sodium sulfate.

16. The formulation of any one of claim 11-15, wherein the formulation comprises about 10 mM to about 750 mM of the salt.

17. The formulation of claim 16, wherein the formulation comprises about 20 mM to about 750 mM of the salt.

18. The formulation of any of claims 1-3, wherein the salt is lithium acetate, lithium aspartate, lithium glutamate, or lithium sulfate.

19. The formulation of claim 18, wherein the salt is lithium acetate.

20. The formulation of claim 18, wherein the salt is lithium aspartate.

21. The formulation of claim 18, wherein the salt is lithium glutamate.

22. The formulation of claim 18, wherein the salt is lithium sulfate.

23. The formulation of any one of claim 18-22, wherein the formulation comprises about 10 mM to about 750 mM of the salt.

24. The formulation of claim 23, wherein the formulation comprises about 20 mM to about 750 mM of the salt.

25. The formulation of any one of claims 1 and 4-24, wherein the buffer is selected from the group consisting of: acetate, succinate, gluconate, histidine, citrate, and phosphate.

26. The formulation of claim 25, wherein the buffer is a histidine buffer.

27. The formulation of claim 25, wherein the buffer is an acetate buffer.

28. The formulation of claim 25, wherein the buffer is a citrate buffer.

29. The formulation of claim 25, wherein the buffer is a phosphate buffer.

30. The formulation of any one of claims 1 and 4-29, wherein the formulation comprises about 1 mM to about 100 mM of the buffer.

31. The formulation of claim 30, wherein the formulation comprises about 1 mM to about 75 mM of the buffer.

32. The formulation of claim 31, wherein the formulation comprises about 1 mM to about 50 mM of the buffer.

33. The formulation of claim 32, wherein the formulation comprises about 1 mM to about 20 mM of the buffer.

34. The formulation of any one of claims 1-33, wherein the formulation has a pH of about 5 to about 6.

35. The formulation of claim 34, wherein the formulation has a pH of about 5.5.

36. The formulation of any one of claims 1-35, wherein the formulation comprises an antibody.

37. The formulation of claim 36, wherein the antibody is a monoclonal antibody.

38. The formulation of claim 37, wherein the monoclonal antibody is a human antibody or a humanized antibody.

39. The formulation of claim 37, wherein the monoclonal antibody has an Fc amino acid substitution that decreases its conformational stability as compared to a similar antibody not including the Fc amino acid substitution.

40. The formulation of claim 37, wherein the monoclonal antibody is an IgG1 or an IgG4 antibody.

41. The formulation of claim 37, wherein the monoclonal antibody is an anti-C—X—C motif chemokine receptor 3 (CXCR3) monoclonal antibody.

42. The formulation of claim 41, wherein the anti-CXCR3 monoclonal antibody comprises a heavy chain comprising SEQ ID NO: 1 and a light chain comprising SEQ ID NO: 2.

43. The formulation of claim 37, wherein the monoclonal antibody is an anti-carcinoembryonic antigen-related cell adhesion molecule 5 (CEACAM5) monoclonal antibody.

44. The formulation of claim 43, wherein the anti-CEACAM5 monoclonal antibody comprises a heavy chain comprising SEQ ID NO: 7 and a light chain comprising SEQ ID NO: 8.

45. The formulation of claim 37, wherein the monoclonal antibody is an anti-carcinoembryonic antigen-related cell adhesion molecule 5 (CEACAM5)-Fc engineered monoclonal antibody.

46. The formulation of claim 45, wherein the anti-CEACAM5-Fc engineered monoclonal antibody comprises a heavy chain comprising SEQ ID NO: 9 and a light chain comprising SEQ ID NO: 10.

47. The formulation of claim 45, wherein the anti-CEACAM5-Fc engineered monoclonal antibody comprises a heavy chain comprising SEQ ID NO: 11 and a light chain comprising SEQ ID NO: 12.

48. The formulation of claim 45, wherein the anti-CEACAM5-Fc engineered monoclonal antibody comprises a heavy chain comprising SEQ ID NO: 13 and a light chain comprising SEQ ID NO: 14.

49. The formulation of any one of claims 1-48, wherein the formulation comprises about 0.1 mg/mL to 400 mg/mL of the antibody or the antigen-binding antibody fragment.

50. The formulation of claim 49, wherein the formulation comprises about 0.1 mg/mL to 250 mg/mL of the antibody or the antigen-binding antibody fragment.

51. The formulation of claim 50, wherein the formulation comprises about 0.1 mg/mL to about 200 mg/mL of the antibody or the antigen-binding antibody fragment.

52. The formulation of claim 51, wherein the formulation comprises about 0.1 mg/mL to about 150 mg/mL of the antibody or the antigen-binding antibody fragment.

53. The formulation of claim 52, wherein the formulation comprises about 0.1 mg/mL to about 100 mg/mL of the antibody or the antigen-binding antibody fragment.

54. The formulation of claim 53, wherein the formulation comprises about 0.1 mg/mL to about 50 mg/mL of the antibody or the antigen-binding antibody fragment.

55. The formulation of claim 54, wherein the formulation comprises about 0.1 mg/mL to about 25 mg/mL of the antibody or the antigen-binding antibody fragment.

56. The formulation of any one of claims 1-55, wherein the formulation has a viscosity of about 1 cP to about 50 cP.

57. The formulation of claim 56, wherein the formulation has a viscosity of about 1 cP to about 40 cP.

58. The formulation of claim 57, wherein the formulation has a viscosity of about 1 cP to about 30 cP.

59. The formulation of claim 58, wherein the formulation has a viscosity of about 1 cP to about 20 cP.

60. The formulation of any one of claims 1-59, wherein the formulation has an osmolality of about 250 mOsm/kg to about 1500 mOsm/kg.

61. The formulation of claim 60, wherein the formulation has an osmolality of about 250 mOsm/kg to about 750 mOsm/kg.

62. The formulation of claim 61, wherein the formulation has an osmolality of about 250 mOsm/kg to about 500 mOsm/kg.

63. The formulation of claim 62, wherein the formulation has an osmolality of about 250 mOsm/kg to about 400 mOsm/kg.

64. The formulation of any one of claims 1-63, wherein the formulation is stable at 25° C. for about 1 hour to about 2 years.

65. The formulation of claim 1-63, wherein the formulation is stable at 40° C. about 1 hour to about 8 weeks.

66. The formulation of any one of claims 1-65, wherein the formulation is suitable for intravenous, intramuscular, or subcutaneous administration.

67. The formulation of claim 66, wherein the formulation is suitable for intravenous administration.

68. The formulation of claim 66, wherein the formulation is suitable for subcutaneous administration.

69. The formulation of any one of claims 1-68, wherein the formulation further comprises one or more of a stabilizer, an anti-oxidant, a metal chelator, a viscosity modifier, an amino acid, and a surfactant.

70. The formulation of claim 69, wherein the stabilizer is fructose, maltose, galactose, glucose, O-mannose, sorbose, lactose, sucrose, trehalose, cellobiose, raffinose, melezitose, a maltodextrin, a dextran, starch, mannitol, xylitol, maltitol, lactitol, glucitol, sucrose, trehalose, raffinose, maltose, sorbitol, mannitol, an amino sugar, sodium chloride, and glycerol.

71. The formulation of claim 69, wherein the antioxidant is methionine, ascorbic acid, or N-acetyl cysteine.

72. The formulation of claim 69, wherein the metal chelator is sodium ethylenediaminetetraacetic acid (EDTA), calcium EDTA, or diethylenetriamine pentaacetate (DTPA).

73. The formulation of claim 69, wherein the viscosity modifier is arginine, histidine, lysine, proline, glycine, or sodium chloride.

74. The formulation of claim 69, wherein the surfactant is selected from the group consisting of: sorbitan monocaprylate, sorbitan monolaurate, sorbitan monopalmitate, sorbitan trioleate, glycerine monocaprylate, glycerine monomyristate, glycerine monostearate, decaglyceryl monostearate, decaglyceryl distearate, decaglyceryl monolinoleate, polyoxyethylene sorbitan monolaurate, polyoxythylene sorbitan monocleate, polyoxyethylene sorbitan monostearate, polyoxyethylene sorbitan monopalmitate, polyoxyethyene sorbitan trioleate, polyoxyethylene sorbitan tristearate, polyoxyethylene sorbitol tetrastearate, polyoxyethylene sorbitol tetraoleate, polyoxyethylene glyceryl monostearate, polyethylene glycol distearate, polyoxyethylene lauryl ether, polyoxyethylene polyoxypropylene glycol, polyoxyethylene polyoxypropylene propyl ether, polyoxyethylene polyoxypropylene cetyl ether, polyoxyethylene nonylphenyl ether, polyoxythylene castor oil, polyoxyethylene hydrogenated castor oil, polyoxyethylene sorbitol beeswax, polyoxyethylene lanolin, polyoxyethylene stearic acid amide, sodium cetyl sulfate, sodium lauryl sulfate, sodium oleyl sulfate, sodium polyoxyethylene lauryl sulfate, sodium lauryl sulfosuccinate ester, lecithin, a glycerophosopholipid, a sphingophospholipid, polysorbate 20, polysorbate 40, polysorbate 60, polysorbate 80, poloxamer 188, triton-X, sodium lauryl sulfate, polyethylene glycol, and propylene glycol.

75. The formulation of claim 69, wherein the amino acid is selected from the group consisting of: arginine, lysine, histidine, proline, ornithine, isoleucine, leucine, alanine, glycine, glutamic acid, and aspartic acid.

76. An injection device comprising a formulation of any one of claims 1-75.

77. A kit comprising one or more vials containing a formulation of any one of claims 1-75.

78. The kit of claim 77, further comprising an injection device for administration of the formulation to a subject in need thereof.

79. A method of making an aqueous antibody formulation, the method comprising mixing or combining:
(i) an antibody or an antigen-binding fragment thereof;
(ii) a buffer;
(iii) a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, and
(iv) a stabilizer;
(v) a surfactant; and
(vi) sterile water,
wherein (i) to (vi) are mixed or combined in amounts sufficient to generate the formulation of any one of claims 1-75.

80. The method of claim 79, further comprising mixing or combining one or more of an antioxidant, a metal chelator, and a viscosity modifier to (i) to (vi).

81. A method of making an aqueous antibody formulation, the method comprising mixing or combining:
(i) an antibody or an antigen-binding fragment thereof;
(ii) a salt selected from the group consisting of: magnesium glutamate, magnesium acetate, magnesium aspartate, magnesium sulfate, arginine acetate, arginine aspartate, arginine glutamate, arginine sulfate, lysine acetate, lysine aspartate, lysine glutamate, lysine sulfate, sodium acetate, sodium aspartate, sodium glutamate, sodium sulfate, lithium acetate, lithium aspartate, lithium glutamate, and lithium sulfate, and
(iv) a stabilizer;
(v) a surfactant; and
(vi) sterile water,
wherein (i) to (v) are mixed or combined in amounts sufficient to generate the formulation of any one of claims 1-75.

82. The method of claim 81, wherein the method does not comprise mixing or combining a buffer with (i) to (v).

83. The method of claim 81 or 82, further comprising mixing or combining one or more of an antioxidant, a metal chelator, and a viscosity modifier to (i) to (vi).

84. A method of treating a subject in need thereof, the method comprising administering to the subject a therapeutically effective amount of a formulation of any one of claims 1-75.


SEQUENCE LISTING (14 sequences)

SEQ ID NO: 1 - protein, 452 aa, Artificial Sequence
  Heavy Chain, Antibody A - Anti-CXCR3-DE (S239D,I332E)- Fc Mutant
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 213 aa, Artificial Sequence
  Light Chain, Antibody A - Anti-CXCR3-DE (S239D,I332E)- Fc Mutant
DIQLTQSPSFLSASVGDRVTITCRASSGVNYLYWYQQKPGKAPKLWIYFTSTLASGVPSRFSGSGSGNEYTLTISSLQPEDFATYYCQQFTSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, Artificial Sequence
  Heavy Chain, Antibody C - Anti-CD38 (Isatuximab) - Wild Type IgG1 Fc
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 214 aa, Artificial Sequence
  Light Chain, Antibody C- Anti-CD38 (Isatuximab) - Wild Type IgG1 Fc
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 449 aa, Artificial Sequence
  Heavy Chain, Antibody B - Anti-CD38-ADE (G236A,S239D,I332E)- Fc Mutant
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 214 aa, Artificial Sequence; identical to SEQ ID NO: 4
  Light Chain, Antibody B - Anti-CD38-ADE (G236A,S239D,I332E)- Fc Mutant

SEQ ID NO: 7 - protein, 449 aa, Artificial Sequence
  Heavy Chain, Anti-CEACAM5 - Wild Type IgG1 Fc
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 214 aa, Artificial Sequence
  Light Chain, Anti-CEACAM5 - Wild Type IgG1 Fc
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 449 aa, Artificial Sequence
  Heavy Chain, Antibody D - Anti-CEACAM5 (S239D) - Fc Mutant
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, Artificial Sequence; identical to SEQ ID NO: 8
  Light Chain, Antibody D - Anti-CEACAM5 (S239D) - Fc Mutant

SEQ ID NO: 11 - protein, 449 aa, Artificial Sequence
  Heavy Chain, Antibody E - Anti-CEACAM5 (I332E) - Fc Mutant
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, Artificial Sequence; identical to SEQ ID NO: 8
  Light Chain, Antibody E- Anti-CEACAM5 (I332E) - Fc Mutant

SEQ ID NO: 13 - protein, 449 aa, Artificial Sequence
  Heavy Chain, Antibody DE - Anti-CEACAM5 (S239D,I332E) - Fc Mutant
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 214 aa, Artificial Sequence; identical to SEQ ID NO: 8
  Light Chain, Antibody DE - Anti-CEACAM5 (S239D,I332E) - Fc Mutant